(19) United States
(12) Patent Application Publication
(10) Pub. No.: US 2009/0130070 A1
(43) Pub. Date: May 21, 2009

(54) METHOD OF TREATMENT

(75) Inventors: Mark Hayes, Queensland (AU); Leila Cuttle, Queensland (AU); Margaretha Kempf, Queensland (AU); John Fraser, Queensland (AU); Roy Kimble, Queensland (AU)

Correspondence Address: PROSKAUER ROSE LLP, 1001 PENNSYLVANIA AVE, N.W.,, SUITE 400 SOUTH, WASHINGTON, DC, 20004, US

(73) Assignee: The University of Queensland, St. Lucia (AU)

(21) Appl. No.: 11/813,139

(22) PCT Filed: Dec. 29, 2005
(86) PCT No.: PCT/AU2005/001965

§ 371 Date: Jan. 9, 2008

(30) Foreign Application Priority Data
Dec. 31, 2004 (AU) 2004907371
Sep. 14, 2005 (AU) 2005905080

(51) Int. Cl. A61K 35/12; A61K 38/17; A61P 17/02

(52) U.S. Cl. 424/93.21; 514/8; 424/93.7

(57) ABSTRACT

The present invention discloses the use of fetuin and fetuin producing agents in methods and compositions for treating burn injuries in animals.

### FIELD OF THE INVENTION

This invention relates generally to the field of tissue remodeling and more particularly to healing of skin tissue that has been subjected to trauma. Still more particularly, the present invention relates to compositions and their use in the treatment of burn injuries in vertebrate animals, and especially mammals including human subjects.

Bibliographic details of the publications referred to by author in this specification are collected at the end of the description.

### BACKGROUND OF THE INVENTION

Skin comprises an outer epidermis which sits over a layer of connective tissue called the dermis and an inner layer called the hypodermis. The connective tissue provides strength, has blood vessels and nerves running through it and contains fibroblasts that secrete an extracellular matrix. The epidermis is a stratified composition comprising keratinocytes. The innermost layer of the epidermis, called the basal layer, houses stem cells that feed the epidermis with replacement keratinocytes as the upper layers fall off. If epidermal cells are damaged, surrounding epidermal cells migrate into the area, proliferate and effectively replace the damaged skin.

The healing process in response to incisional or excisional wounds has been studied in some depth. When tissues are subjected to incisional or excisional injury, the body responds by initiating complex sets of saving responses that result in a healing process which ideally culminates in the replacement of damaged tissue with tissue of the same type i.e., epithelial and dermal tissue for skin (dermal) injuries.

Many biological molecules whose role involves transmitting signals between cells and influencing cellular trafficking are thought to have a role in the control of wound healing. For example, Epidermal Growth Factor (EGF), Fibroblast Growth Factor (FGF), Wnt, Hedgehog, Notch, Integrins, Bone Morphogenic Protein (BMP)/Transforming Growth Factor Beta (TGF-β) and Platelet Derived Growth Factor (PDGF) have all been have been implicated in wound healing. The involvement of various growth factors and cytokines factors in wound healing is reviewed by Werner et al (Physio Rev 83:835-870, 2003).

An incisional or excisional wound to the dermis involves rupture of blood vessels and bleeding, which initiates platelet activation, clotting, platelet degranulation and an influx of inflammatory cells into the wound site in response to soluble factors. These cells initially comprise mostly neutrophils, followed by macrophages and lymphocytes. Soluble factors, such as TGF-βs and Platelet Derived Growth Factor (PDGF) are produced by the cells of the skin and blood, including monocytes and macrophages and promote fibroblast migration into the wound. Fibroblasts then proliferate and differentiate into myofibroblasts and form an initial thick coating over the wound. Proteins which will form the extracellular matrix, such as collagen, fibronectin and actin are produced by fibroblasts and secreted. Differences in the amount and organization of the extracellular matrix is thought to be an important determinant of the fate of the wound to either heal with a scar or in a scarless fashion. Once tissue continuity (wound closure or contraction) has been achieved by the deposition of extracellular matrix, remodeling of the wound site takes place with continued synthesis of extracellular matrix by fibroblasts and proteolytic breakdown by matrix metalloproteinases (MMPs).

If the healing process is imperfect, perhaps because it is perturbed through infection or the damage is so severe that the wound cannot be perfectly healed, the end point in the healing process is a scar. A scar is the general term to describe a wound site which has not healed perfectly and which comprises fibrotic tissue rather than tissue of the same type as the pre-damaged tissue. Scar tissue generally consists of fibroblastic cells and extracellular matrix comprising mainly fibrinogen, actin and collagen proteins. The ability of the wound to contract is due to the presence of actin filaments synthesized by myofibroblasts that form stress fibers within the wound which can contact and close the wound. In some situations scar tissue can become hypertrophic or keloid scars can form which spread to adjacent normal tissue.

In general, if healing is impaired for example by sepsis or hypoxia (impaired oxygen perfusion) excessive inflammatory responses can prevent wound closure, impair the deposition of extracellular matrix and impair remodeling, leading to a scar. Specifically, activated macrophages increase levels of inflammatory cytokines which can lead to increased MMP synthesis and decreased collagen synthesis.

Burn injuries are recognized as a major cause of death in the relatively fit and young. There have been major advances in the treatment of patients with severe burn injury and the mortality associated with burns has dropped dramatically. However similar improvements are not seen in the morbidity associated with post-burn scarring. Scar tissue does not expand with a growing child and this can lead to chronic loss of joint movement. Scarring, particularly in children, can be seen as a disease that affects psycho-social development, osteopenia, body temperature regulation (due to reduced efficiency of perspiration). Scarring requires repeated operations for contracture release, as well as the discomfort associated with continued physiotherapy and occupational therapy. Available treatments for burn injuries are limited. Specifically, treatment comprises prolonged wearing of pressure garments which often have poor results even when compliance is achieved, steroid injections which are associated with their own morbidity particularly in the growing child and repeated grafting that also causes scarring.

A burn wound generally has significant horizontal and vertical components and there is generally a much larger loss of tissue than in the case of an excisional or incisional wound. An incisional wound is created, for example, by a sharp knife and following injury the stimulus is ended. In contrast in burns the tissue destruction does not cease on the removal of the heat source—cell injury occurs de novo long after the removal of the heat source. Burn injuries can lead to hypoxia and immunosuppression in the burn area that can inhibit many of the normal healing mechanisms.

There is a large array of factors and events that determine whether and how a wound heals. A review article by Ferguson et al (Phil. Trans. R. Soc. Lond. B (2004) 359, 839-850) discusses the use of neutralizing antibodies to PDGF, TGF-β1 or TGF-β2 generally to enhance wound healing in the case of incisional or excisional wounds but not burn wounds. The authors suggest that TGF-β3 is critically important for stimulating fibroblast migration into a healing wound and reduced scarring. They suggest that all isoforms of TGF-β cause fibroblast proliferation indicating that this activity is not required for wound healing. This approach is based on the observation that fetal wounds heal without a scar and are associated with low levels of TGF-β1 and TGF-β2 and high levels of TGF-β3. Burn wounds are not discussed in any detail although they are merely cited as an example of a traumatic injury requiring rapid attention (page 843, first full paragraph). However, other studies suggest that exogenous TGF-βs stimulate re-epithelialization and granulation tissue formation consistent with their endogenous role (Werner et al, supra) and thus the roles and effects of TGF-βs are by no means clear. In particular, it is unclear as to whether TGF-β modulation by itself provides the proposed panacea for dermal injury.

Specific TGF-β antagonists have been reported to enhance the rate of re-epithelialization and decrease the amount of wound contraction and fibrosis (Huang J. S. et al FASEB J (21 Jun., 2002) 10.1096/fj.02-0103fje). Huang et al. describe the effects of topical application of a peptide antagonist of TGF-1 on excision and burn wounds over time in a pig model. Peptide treated wounds showed an increased rate of re-epithelialization in burn wounds, although the extent of re-epithelialization by Day 14 in tests and controls was about the same. Also, the peptide reduced the extent of wound contraction (closure). In another study, TGF-β1 deficient mice showed severely impaired wound repair with decreased re-epithelialization and granulation and enhanced inflammatory cell infiltrates (Brown et al Wound Repair Regen 3:25-36, 1995).

Fetuin (also called Fetuin A, alpha2-Heremans-Schmid-glycoprotein (AHSG) and Countertrypsin) is a glycoprotein produced in the liver and by cells of the monocyte/macrophage lineage and is found in high levels in plasma. It is an acidic glycoprotein with three N-linked and three O-linked oligosaccharide chains. Fetuin has been reported to interact with a large number of different polypeptides and cells. It appears to regulate inflammatory responses and particularly the innate immune system responses. Fetuin binds strongly to TGF-β1 and TGF-β2 and weakly to TGF-β3. Fetuin binds strongly to bone morphogenic proteins BMP-2, 4 & 6 (Demetriou M. et al., 1996, J. Biol. Chem., 271(22):12755-12761; Szweras M. et al., 2002, J. Biol. Chem., 277(22):19991-19997). Fetuin also modifies macrophage responses, opsonizes apoptotic cells and reduces inflammatory responses (Wang H. et al., 1998, Proc. Natl. Acad. Sci. USA, 95(24):14429-14434). Fetuin also appears to activate matrix metalloproteinases MMP-3 and 9 (Tajirian T. et al., 2000, J. Cell. Physiol., 185(2):174-183; Leite-Browning M. L. et al., 2002, Int. J. Oncol., 21(5):965-971) and is a member of the cystatin serine protease inhibitor superfamily (Brown W. M. et al., 1997, Protein Sci., 6(1):5-12).

U.S. Pat. No. 5,981,483 in the name of Dennis discloses that fetuin and thyroglobulin bind to members of the TGF-β superfamily. Dennis discloses that fetuin binds antagonistically to TGF-β1 which promotes lung epithelial cell proliferation in vitro. Based on the known activities of certain TGF-β superfamily members, Dennis proposes a very wide range of applications for agonists and antagonists of TGF-β. In particular, agonists are proposed for wound healing while antagonists are proposed to stimulate an immune response and reduce the deposition of extracellular matrix. Thus, it appears that fetuin would be expected to promote epithelial cell growth, decrease wound healing, stimulate an immune response and reduce the deposition of extracellular matrix. Accordingly, Dennis teaches away from using fetuin in wound healing.

There is clearly a need for greater understanding of dermal injuries and for methods and compositions for their treatment.

### SUMMARY OF THE INVENTION

In work leading up to the present invention, it was discovered that fetuin is present in fetal tissue at much greater levels that in post-natal including adult tissue. Additionally, the presence of fetuin in the skin was found to coincide with the occurrence of scar-free burn healing in a fetal animal model of burn injury and was shown to be effective in promoting wound healing. Further, fetuin was shown to be markedly more effective than either thyroglobulin or a TGF-β1 inhibitor in enhancing healing of dermal injury and in promoting wound closure and healing.

Accordingly, one aspect of the present invention provides methods for treating a dermal injury in a subject. The methods generally comprise administering to the subject an effective amount of a fetuin polypeptide or an agent from which a fetuin polypeptide is producible. As described further herein the term “fetuin polypeptide” includes and encompasses all naturally-occurring forms of fetuin (AHSG) as well as biologically active portions thereof and variants or derivatives of these. Dermal injuries include excisional, incisional and burn injuries. Dermal injuries also include injury as a result of infection and/or inflammation. Burn injuries include, for example, thermal, frictional or chemical burns. In some embodiments, the fetuin polypeptide or the agent from which the fetuin polypeptide is producible is prepared with one or more pharmaceutically acceptable carriers, diluents and/or excipients. In other embodiments, the fetuin polypeptide or the agent from which the fetuin polypeptide is producible is applied, attached or otherwise associated with a medical device or medical material such as, for example, a suture, synthetic or substitute skin or a gauze or dressing. In some embodiments, the agent from which the fetuin polypeptide is producible is a cell which is capable of secreting a fetuin polypeptide. For example, cells are cultured in vitro and administered as a suspension or in the form of a graft or skin substitute. In other embodiments, the agent from which the fetuin polypeptide is producible is a polynucleotide (nucleic acid molecule) encoding the fetuin polypeptide. In some embodiments, the methods further comprise debriding the dermal injury to remove devitalised tissue. In illustrative examples, the debriding is carried out prior to or simultaneous with the administration of the fetuin polypeptide or the fetuin polypeptide-producing agent. In some embodiments, the polypeptide or agent is administered locally, such as, for example, by administering topically, transversely, intradermally, cutaneously, subcutaneously at or near to the site of dermal injury.

In another aspect, the present invention provides the use of a fetuin polypeptide or an agent from which a fetuin polypeptide is producible in the manufacture of a medicament for treating a dermal injury.

In still another aspect, the invention provides kits for treating a dermal injury, comprising a fetuin polypeptide or an agent from which a fetuin polypeptide is producible and a debridement agent.

### BRIEF DESCRIPTION OF THE DRAWINGS

FIG. 1 is a photographic representation of silver stained 2-D gels showing the reproducibility of the results of 2-D analysis in triplicate gels of fetal control (day 80 gestation) samples.

FIG. 2 is a photographic representation of a silver stained 2-D gel, 14 days after burn injury indicating (with arrow) major differentially expressed protein (fetuin). Differentially expressed proteins were identified by scanning the silver stained gels, assigning them different colors and overlaying them using Adobe PhotoShop. A transparency of the gel showing spots of interest was printed and used to identify spots for extraction. The major differentially expressed protein is present at much higher concentration in fetal control protein extracts than in the lamb control. It also has a much higher concentration in the 14 day post burn fetal sample than in the 14 day post burn lamb sample. This protein spot was excised from the replicate gels for both fetus and lamb, trypsin digested and subjected to MALDI TOF and MALDI MS/MS analysis and was identified with high reliability as ovine Fetuin A.

FIG. 3 is a photographic representation of silver stained 2-D gels showing fetal and lamb control skin (left side) and fetal and lamb burned skin, 14 days after the burn (right side). It appears as though fetuin (marked by arrows) may be upregulated after burn injury. However, the upregulation may be due to a fluctuation during gestation.

FIG. 4 is a photographic representation of silver stained 2-D gels. To identify whether the fetuin protein concentration rose in response to the burn injury, 2D gels were run on skin extracts from normal unburned skin from a burned animal and a fetus of the same gestation as the 14 day post burn animal which had not been burned or operated on (day 94 gestation). These results show that the level of fetuin in the skin is varies according to the duration of gestation rather than a response to burn or the operation. Gels of samples from control fetal skin on a burned animal at day 14 after burn (left) and from normal fetal skin on a sham operated animal at 14 days (right). The fetuin spot is clearly as large on these gels as it is in the fetal burn day 14 gels, indicating that the high levels of fetuin are due to gestational age changes rather than an upregulation in response to burn.

FIG. 5 is a photographic representation of silver stained 2-D gels demonstrating changes in the levels of fetuin with gestational age. Gels were run from samples 60 days after burn, but from normal skin. This tissue is from 140 days gestation (term is day 145-150). Gels of samples from day 60 normal skin in fetus (left) and lamb (right). The amount of fetuin is very small at this time point, compared to earlier gestational time points.

FIG. 6 is a photographic representation showing a cross section of skin after immunohistochemistry to detect fetuin in fetal day 14 burn skin (left) and lamb burn day 1 skin (right). Fetuin is present in higher levels in the fetus and is predominantly in blood vessels, but also present throughout the dermal and epidermal tissue. Magnification ×100.

FIG. 7 is a photographic representation showing in situ hybridization of probes to fetuin in fetal and lamb skin 1 day after burn, compared to positive control tissue. Fetuin is not visible in the fetal or lamb tissue, but stains positively in some cells in the liver. Magnification ×40.

FIG. 8 is a photographic representation of a shaved lamb flank to which fetuin has been applied in various different carrier compositions to burn injuries.

FIG. 9 is a photographic representation of a cross section of epidermis showing the penetration of the labeled fetuin in a water solution. Labeled fetuin can be seen on the epidermis and at the end of hair follicles. Magnification ×100.

FIG. 10 is a photographic representation of a cross section of epidermis showing the penetration of the labeled fetuin in an aqueous cream. Magnification ×100.

FIG. 11 is a photographic representation of a cross section of epidermis showing the penetration of the labeled fetuin in an aqueous cream. Magnification ×400.

FIG. 12 is a graphical representation showing the levels of αSMA in fetal and lamb skin in burnt and control animals.

FIG. 13 is a graphical representation showing the levels of TGF-β in fetal and lamb skin in burnt and control animals.

FIG. 14 is a graphical representation showing the effect of thyroglobulin on wound closure.

FIG. 15 is a graphical representation showing the effect of a TGF-β1 receptor blocker on wound closure.

FIG. 16 is a graphical representation showing the effect of fetuin on wound closure.

FIG. 17 is a photographic representation showing delivery of fetuin compositions to a porcine burn model with and without prior debridement. Fluorescent green color is the labeled protein while the blue color is DAPI staining of nuclear DNA. Arrows mark the skin surface.

FIG. 18 is a diagrammatic representation showing an alignment of fetuin polypeptides from, human, chimpanzee, mouse, rat, cattle, sheep, pig and guinea pig.

### DETAILED DESCRIPTION OF THE PREFERRED EMBODIMENTS

### 1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

Each embodiment described herein is to be applied mutatis mutandis to each and every other embodiment unless specifically stated otherwise.

The articles “a” and “an” are used herein to refer to one or to more than one (i.e. to at least one) of the grammatical object of the article. By way of example, “a cell” means one cell or more than one cell.

As used herein, the term “about” refers to a quantity, level, value, percentage, dimension, size, or amount that varies by as much as 30%, 20% or 10% to a reference quantity, level, value, percentage, dimension, size, or amount.

The term “agent” in the context of healing wounds (e.g., “wound healing agent” or “agent that modulates the level of activity of one or more members of the TGF-β family”) refers to a compound that induces the desired pharmacological and/or physiological effect. The term also encompasses pharmaceutically acceptable and pharmacologically active ingredients of those compounds specifically mentioned herein including but not limited to salts, esters, amides, prodrugs, active metabolites, analogs and the like. When the above term is used, then it is to be understood that this includes the active agent per se as well as pharmaceutically acceptable, pharmacologically active salts, esters, amides, prodrugs, metabolites, analogs, etc. The term “agent” is not to be construed narrowly but extends to small molecules and macromolecules including but not limited to proteinaceous molecules such as peptides, polypeptides and proteins as well as genetic molecules such as RNA, DNA and mimetics and chemical analogs thereof as well as cellular agents. The term “agent” in the phrase “agent from which fetuin polypeptide is producible” includes a cell which is capable of producing and secreting fetuin polypeptide as well as a polynucleotide comprising a nucleotide sequence that encodes a fetuin polypeptide. In illustrative examples of this type, the fetuin-encoding nucleotide sequence is operably connected to a regulatory element in a nucleic acid construct Thus, the term “agent” extends to nucleic acid constructs including vectors such as viral or non-viral vectors, expression vectors and plasmids for expression in and secretion in a range of cells.

“Analogs” contemplated herein include, but are not limited to, modification to side chains, incorporating of unnatural amino acids and/or their derivatives during peptide, polypeptide or protein synthesis and the use of crosslinkers and other methods which impose conformational constraints on the proteinaceous molecule or their analogs.

By “biologically active portion” is meant a portion of a full-length parent peptide or polypeptide which portion retains an activity of the parent molecule. As used herein, the term “biologically active portion” includes deletion mutants and peptides, for example of at least about 8, 9, 10, 11, 12, 13, 14, 15, 16, 17, 18, 19, 20, 21, 22, 23, 24, 25, 30, 40, 50, 60, 70, 80, 90, 100, 120, 150, 300, 350 contiguous amino acids (and every integer in between), which comprise an activity of a reference fetuin polypeptide. One example of a biologically active portion is a form of the polypeptide without a signal or leader sequence. Portions of this type may be obtained through the application of standard recombinant nucleic acid techniques or synthesized using conventional liquid or solid phase synthesis techniques. For example, reference may be made to solution synthesis or solid phase synthesis as described, for example, in Chapter 9 entitled “Peptide Synthesis” by Atherton and Shephard which is included in a publication entitled “Synthetic Vaccines” edited by Nicholson and published by Blackwell Scientific Publications. Alternatively, peptides can be produced by digestion of a peptide or polypeptide of the invention with proteinases such as endoLys-C, endoArg-C, endoGlu-C and staphylococcus V8-protease. The digested fragments can be purified by, for example, high performance liquid chromatographic (HPLC) techniques. Recombinant nucleic acid techniques can also be used to produce such portions. The biological activities of portions are tested in vivo and/or in vitro.

Reference to a “burn” includes reference to a thermal, frictional or chemical injury to the skin. Thermal burns may be caused by hot or cold liquids, solids, gases or mixtures of these, the sun and other forms of radiation, light, lasers, including during surgical or diagnostic procedures or operations. Chemical burns may be caused, for example, by caustic chemicals or strong acids. First degree burns are superficial, appearing wet, pink and blistered. Second degree burns range from mild to severe with destruction of the entire epidermis and part of the dermis. In third degree burns, the epidermis and the dermis are destroyed leaving insufficient cells to allow self-healing.

By “cell” is meant any prokaryotic or eukaryotic cell. The term “dermal cell” is to be construed broadly to include any cell which is capable of contributing to forming or associating with skin tissue including neutrophils, monocytes, macrophages, fibroblasts, lymphocytes, adipocytes, epithelial cells, myofibroblasts, melanocytes, keratinocytes, stem cells or their progenitors. A syngeneic cell is genetically identical to the subject or is genetically compatible to minimise any immune response.

By “co-administered” is meant simultaneous administration in the same formulation or in two different formulations via the same or different routes or sequential administration by the same or different routes. For example, the subject composition may be administered together with another healing agent in order to enhance its effects. By “sequential” administration is meant a time difference of from seconds, minutes, hours or days between the administration of the two types of molecules. These molecules may be administered in any order.

“Complementary” as used herein, refers to the capacity for precise pairing between two nucleobases of an oligomeric compound. For example, if a nucleobase at a certain position of an oligonucleotide (an oligomeric compound), is capable of hydrogen bonding with a nucleobase at a certain position of a target nucleic acid, said target nucleic acid being a DNA, RNA, or oligonucleotide molecule, then the position of hydrogen bonding between the oligonucleotide and the target nucleic acid is considered to be a complementary position. The oligonucleotide and the further DNA, RNA, or oligonucleotide molecule are complementary to each other when a sufficient number of complementary positions in each molecule are occupied by nucleobases which can hydrogen bond with each other.

Throughout this specification, unless the context requires otherwise, the words “comprise,” “comprises” and “comprising” will be understood to imply the inclusion of a stated step or element or group of steps or elements but not the exclusion of any other step or element or group of steps or elements.

As used herein, the term “dermal injury” refers to any opening in the skin and associated mucosa or epithelial linings, most such openings generally being associated with exposed, raw or abraded tissue. There are no limitations as to the type of dermal injuries that can be treated in accordance with this invention, such injuries include, but are not limited to, first, second, and third degree burns; surgical incisions and excisions, including those of cosmetic surgery; and wounds including abrasions, lacerations, incisions, perforations and penetrations.

By “derivative” is meant a polypeptide that has been derived from the basic sequence by modification, for example by conjugation or complexing with other chemical moieties or by post-translational modification techniques as would be understood in the art. The term “derivative” also includes within its scope alterations that have been made to a fetuin polypeptide including additions, or deletions that provide for functionally equivalent molecules.

Reference herein to “enhancing wound healing” includes improved healing of a dermal injury as measured by reference to any one or more of the following factors; re-epithelialization, granulation, inflammation, infection, remodelling, scar formation, wound contraction, the strength or resilience of the repairing tissue and the like.

By “effective amount,” in the context of treating a dermal injury is meant the administration of that amount of active to a subject, either in a single dose or as part of a series or slow release system, that is effective for treatment. The effective amount will vary depending upon the health and physical condition of the subject and the taxonomic group of individual to be treated, the formulation of the composition, the assessment of the medical situation, and other relevant factors. It is expected that the amount will fall in a relatively broad range that can be determined through routine trials.

The terms “expression” or “gene expression” refer to either production of RNA message or translation of RNA message into proteins or polypeptides. Detection of either types of gene expression in use of any of the methods described herein are part of the invention.

By “expression vector” is meant any autonomous genetic element capable of directing the transcription of a polynucleotide contained within the vector and suitably the synthesis of a peptide or polypeptide encoded by the polynucleotide. Such expression vectors are known to practitioners in the art.

The term “gene” as used herein refers to any and all discrete coding regions of the cell's genome, as well as associated non-coding and regulatory regions. The gene is also intended to mean the open reading frame encoding specific polypeptides, introns, and adjacent 5′ and 3′ non-coding nucleotide sequences involved in the regulation of expression. In this regard, the gene may further comprise control signals such as promoters, enhancers, termination and/or polyadenylation signals that are naturally associated with a given gene, or heterologous control signals. The DNA sequences may be cDNA or genomic DNA or a fragment thereof. The gene may be introduced into an appropriate vector for extrachromosomal maintenance or for integration into the host.

“Hybridization” is used herein to denote the pairing of complementary nucleotide sequences to produce a DNA-DNA hybrid or a DNA-RNA hybrid. Complementary base sequences are those sequences that are related by the base-pairing rules. In DNA, A pairs with T and C pairs with G. In RNA U pairs with A and C pairs with G. In this regard, the terms “match” and “mismatch” as used herein refer to the hybridization potential of paired nucleotides in complementary nucleic acid strands. Matched nucleotides hybridize efficiently, such as the classical A-T and G-C base pair mentioned above. Mismatches are other combinations of nucleotides that do not hybridize efficiently. In the present invention, the preferred mechanism of pairing involves hydrogen bonding, which may be Watson-Crick, Hoogsteen or reversed Hoogsteen hydrogen bonding, between complementary nucleoside or nucleotide bases (nucleobases) of the strands of oligomeric compounds. For example, adenine and thymine are complementary nucleobases which pair through the formation of hydrogen bonds. Hybridization can occur under varying circumstances as known to those of skill in the art.

The phrase “hybridizing specifically to” and the like refer to the binding, duplexing, or hybridizing of a molecule only to a particular nucleotide sequence under stringent conditions when that sequence is present in a complex mixture (e.g., total cellular) DNA or RNA.

Reference to the terms “inhibit” or “inhibition” of TGFβ activity includes completely or partially and directly or indirectly, inhibiting or reducing or down modulating all or part of one or more activities of one or more members of the TGFβ superfamily such as inhibiting cell proliferation in the burn wound, or inhibiting signal transduction initiated by TGF-β. The TGF-β superfamily notably comprises the bone morphogenic proteins (BMPs). Inhibition may be achieved by directly or indirectly reducing the level or activity of TGF-β or its mediators or receptors in genetic or proteinaceous form. As known to those of skill in the art, TGFβ activity may be reduced by modulating transcriptional, post-transcriptional, translational events, promoter or repressor level, function or activity. Signal transduction mediators for the TGF-β family include SMAD2 and SMAD3. Activated SMADs form a complex with, for example SMAD4 and the stability of the complex in the nucleus can influence the cellular response to TGF-β. Assays for testing for the level or activity of TGF-β are routinely employed by those of skill in the art, such as quantifying TGF-β using specific antibody assays or assays for receptor signaling or other cellular effects such as cell growth or differentiation or activation of TGF-β responsive genes.

By “isolated” is meant material that is substantially or essentially free from components that normally accompany it in its native state. For example, an “isolated polynucleotide”, as used herein, refers to a polynucleotide, isolated from the sequences which flank it in a naturally-occurring state, e.g., a DNA fragment which has been removed from the sequences that are normally adjacent to the fragment. Alternatively, an “isolated peptide” or an “isolated polypeptide” and the like, as used herein, refer to in vitro isolation and/or purification of a peptide or polypeptide molecule from its natural cellular environment, and from association with other components of the cell. Without limitation, an isolated polynucleotide, peptide, or polypeptide can refer to a native sequence that is isolated by purification or to a sequence that is produced by recombinant or synthetic means.

By “matrix metalloproteinase” or “MMP” is meant members of a large family of zinc-dependent proteases expressed by cells of the skin and immune system in response to growth factors (cytokines) or noxious stimuli and which are involved, in their active form, in tissue remodeling by breaking down matrix proteins such as collagens, fibrinogens proteoglycans, elastins, decorins and actins. The levels or activity of MMPs can be detected quantitatively or qualitatively for example by polymerase chain reaction (PCR) with primers specific for specific MMP mRNAs, by immunohistochemistry, ELISA or assays which detect MMP proteolytic activity.

By “modulation” or “modulator” in relation to a particular target is meant directly or indirectly up-regulating or down-regulating the level or activity of the target. For example, TGFβ1 may be down-regulated by inhibiting the protease activity which activates the inactivated TGFβ1 pre-pro-protein.

The term “operably connected” or “operably linked” as used herein means placing a structural gene under the regulatory control of a promoter, which then controls the transcription and optionally translation of the gene. In the construction of heterologous promoter/structural gene combinations, it is generally preferred to position the genetic sequence or promoter at a distance from the gene transcription start site that is approximately the same as the distance between that genetic sequence or promoter and the gene it controls in its natural setting; i.e., the gene from which the genetic sequence or promoter is derived. As is known in the art, some variation in this distance can be accommodated without loss of function. Similarly, the preferred positioning of a regulatory sequence element with respect to a heterologous gene to be placed under its control is defined by the positioning of the element in its natural setting; i.e., the genes from which it is derived.

The terms “polynucleotide,” “genetic material,” “genetic forms,” “nucleic acids” and “nucleotide sequence” include RNA, cDNA, genomic DNA, synthetic forms and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog (such as the morpholine ring), internucleotide modifications such as uncharged linkages (e.g. methyl phosphonates, phosphotriesters, phosphoamidates, carbamates, etc.), charged linkages (e.g. phosphorothioates, phosphorodithioates, etc.), pendent moieties (e.g. polypeptides), intercalators (e.g. acridine, psoralen, etc.), chelators, alkylators and modified linkages (e.g. α-anomeric nucleic acids, etc.). Also included are synthetic molecules that mimic polynucleotides in their ability to bind to a designated sequence via hydrogen binding and other chemical interactions. Such molecules are known in the art and include, for example, those in which peptide linkages substitute for phosphate linkages in the backbone of the molecule. RNA forms of the genetic molecules of the present invention are generally mRNA or iRNA including siRNAs. The genetic form may be in isolated form or integrated with other genetic molecules such as vector molecules and particularly expression vector molecules. The terms “nucleotide sequence”, “polynucleotide” and “nucleic acid molecule” used herein interchangeably and encompass polynucleotides in which one or more nucleotides have been added or deleted, or replaced with different nucleotides. In this regard, it is well understood in the art that certain alterations inclusive of mutations, additions, deletions and substitutions can be made to a reference nucleotide sequence whereby the altered polynucleotide retains the biological function or activity of the reference polynucleotide. The terms “polynucleotide variant” and “variant” refer to polynucleotides displaying substantial sequence identity with a reference polynucleotide sequence or polynucleotides that hybridize with a reference sequence under stringent conditions that are defined hereinafter. These terms also encompass polynucleotides in which one or more nucleotides have been added or deleted, or replaced with different nucleotides. In this regard, it is well understood in the art that certain alterations inclusive of mutations, additions, deletions and substitutions can be made to a reference polynucleotide whereby the altered polynucleotide retains a biological function or activity of the reference polynucleotide. The terms “polynucleotide variant” and “variant” also include naturally-occurring allelic variants for example several different allelic variants of AHSG have been described including AHS1 and AHS2.

The terms “polypeptide,” “proteinaceous molecule,” “peptide” and “protein” are used interchangeably herein to refer to a polymer of amino acid residues and to variants and synthetic analogues of the same. Thus, these terms apply to amino acid polymers in which one or more amino acid residues is a synthetic non-naturally-occurring amino acid, such as a chemical analogue of a corresponding naturally-occurring amino acid, as well as to naturally-occurring amino acid polymers. These terms do not exclude modifications, for example, glycosylations, aceylations, phosphorylations and the like. Soluble forms of the subject proteinaceous molecules are particularly useful. Included within the definition are, for example, polypeptides containing one or more analogs of an amino acid including, for example, unnatural amino acids or polypeptides with substituted linkages. The term “polypeptide variant” refers to polypeptides which are distinguished from a reference polypeptide by the addition, deletion or substitution of at least one amino acid residue. In certain embodiments, one or more amino acid residues of a reference polypeptide are replaced by different amino acids. It is well understood in the art that some amino acids may be changed to others with broadly similar properties without changing the nature of the activity of the polypeptide (conservative substitutions) as described hereinafter.

By “obtained from” means derived from, either directly or indirectly.

By “regulatory element” or “regulatory sequence” is meant nucleic acid sequences (e.g., DNA) necessary for expression of an operably linked coding sequence in a particular host cell. The regulatory sequences that are suitable for prokaryotic cells for example, include a promoter, and optionally a cis-acting sequence such as an operator sequence and a ribosome binding site. Control sequences that are suitable for eukaryotic cells include promoters, polyadenylation signals, transcriptional enhancers, translational enhancers, leader or trailing sequences that modulate mRNA stability, as well as targeting sequences that target a product encoded by a transcribed polynucleotide to an intracellular compartment within a cell or to the extracellular environment.

The term “sequence identity” as used herein refers to the extent that sequences are identical on a nucleotide-by-nucleotide basis or an amino acid-by-amino acid basis over a window of comparison. Thus, a “percentage of sequence identity” is calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by an appropriate method. For example, sequence identity analysis may be carried out using the DNASIS computer program (Version 2.5 for windows; available from Hitachi Software engineering Co., Ltd., South San Francisco, Calif., USA) using standard defaults as used in the reference manual accompanying the software.

“Similarity” refers to the percentage number of amino acids that are identical or constitute conservative substitutions as defined in Table A below. Similarity may be determined using sequence comparison programs such as GAP (Deveraux et al. 1984, Nucleic Acids Research 12, 387-395). In this way, sequences of a similar or substantially different length to those cited herein might be compared by insertion of gaps into the alignment, such gaps being determined, for example, by the comparison algorithm used by GAP.

Terms used to describe sequence relationships between two or more polynucleotides or polypeptides include “reference sequence”, “comparison window”, “sequence identity”, “percentage of sequence identity” and “substantial identity”. A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., “Current Protocols in Molecular Biology”, John Wiley & Sons Inc, 1994-1998, Chapter 15.

“Stringency” as used herein refers to the temperature and ionic strength conditions, and presence or absence of certain organic solvents, during hybridization. The higher the stringency, the higher will be the observed degree of complementarity between sequences.

“Stringent conditions” as used herein refers to temperature and ionic conditions under which only polynucleotides having a high proportion of complementary bases, preferably having exact complementarity, will hybridize. The stringency required is nucleotide sequence dependent and depends upon the various components present during hybridization, and is greatly changed when nucleotide analogues are used. Generally, stringent conditions are selected to be about 10° C. to 20° C. less than the thermal melting point (Tm) for the specific sequence at a defined ionic strength and pH. The Tm is the temperature (under defined ionic strength and pH) at which 50% of a target sequence hybridizes to a complementary probe.

It will be understood that an polynucleotide will hybridize to a target sequence under at least low stringency conditions, preferably under at least medium stringency conditions and more preferably under high stringency conditions. Reference herein to low stringency conditions include and encompass from at least about 1% v/v to at least about 15% v/v formamide and from at least about 1 M to at least about 2 M salt for hybridization at 42° C., and at least about 1 M to at least about 2 M salt for washing at 42° C. Low stringency conditions also may include 1% Bovine Serum Albumin (BSA), 1 mM EDTA, 0.5 M NaHPO4 (pH 7.2), 7% SDS for hybridization at 65° C., and (i) 2×SSC, 0.1% SDS; or (ii) 0.5% BSA, 1 mM EDTA, 40 mM NaHPO4 (pH 7.2), 5% SDS for washing at room temperature. Medium stringency conditions include and encompass from at least about 16% v/v to at least about 30% v/v formamide and from at least about 0.5 M to at least about 0.9 M salt for hybridization at 42° C., and at least about 0.5 M to at least about 0.9 M salt for washing at 42° C. Medium stringency conditions also may include 1% Bovine Serum Albumin (BSA), 1 mM EDTA, 0.5 M NaHPO4 (pH 7.2), 7% SDS for hybridization at 65° C., and (i) 2×SSC, 0.1% SDS; or (ii) 0.5% BSA, 1 mM EDTA, 40 mM NaHPO4 (pH 7.2), 5% SDS for washing at 42° C. High stringency conditions include and encompass from at least about 31% v/v to at least about 50% v/v formamide and from at least about 0.01 M to at least about 0.15 M salt for hybridization at 42° C., and at least about 0.01 M to at least about 0.15 M salt for washing at 42° C. High stringency conditions also may include 1% BSA, 1 mM EDTA, 0.5 M NaHPO4 (pH 7.2), 7% SDS for hybridization at 65° C., and (i) 0.2×SSC, 0.1% SDS; or (ii) 0.5% BSA, 1 mM EDTA, 40 mM NaHPO4 (pH 7.2), 1% SDS for washing at a temperature in excess of 65° C. Other stringent conditions are well known in the art. A skilled addressee will recognize that various factors can be manipulated to optimize the specificity of the hybridization. Optimization of the stringency of the final washes can serve to ensure a high degree of hybridization. For detailed examples, see CURRENT PROTOCOLS IN MOLECULAR BIOLOGY (supra) at pages 2.10.1 to 2.10.16 and MOLECULAR CLONING. A LABORATORY MANUAL (Sambrook, et al., eds.) (Cold Spring Harbor Press 1989) at sections 1.101 to 1.104.

“Subjects” contemplated in the present invention include any animal of commercial or humanitarian interest including conveniently, primates, livestock animals including fish and birds, laboratory test animals, companion animals, or captive wild animals. In some embodiments the subject is a mammalian animal. In other embodiments, the subject is a human subject. The present composition and methods have applications in human and veterinary medicine, domestic or wild animal husbandry, cosmetic or aesthetic treatments for the skin after injury.

By “substantially complementary” it is meant that an oligonucleotide or a subsequence thereof is sufficiently complementary to hybridize with a target sequence. Accordingly, the nucleotide sequence of the oligonucleotide or subsequence need not reflect the exact complementary sequence of the target sequence. In a preferred embodiment, the oligonucleotide contains no mismatches and with the target sequence.

The terms “treatment” or “enhancing healing” or “therapy” are used interchangeably in their broadest context and include any measurable or statistically significant change in one or more symptoms or frequency of one or more assessable indications of effective wound healing. Treatment with the present composition may be at any time from when the injury is initially sustained to during or in conjunction with subsequent surgical or microsurgical procedures to enhance dermal healing.

The TGFβ superfamily includes, without limitation, TGFβ and BMP molecules such as TGF-β1, TGF-β2, TGF-β3, TGF-β4, BMP2, BMP3 and BMP4 and fragments, variants or derivatives thereof.

By “vector” is meant a polynucleotide molecule, suitably a DNA molecule derived, for example, from a plasmid, bacteriophage, yeast, virus, mammal, avian, reptile or fish into which a polynucleotide can be inserted or cloned. A vector preferably contains one or more unique restriction sites and can be capable of autonomous replication in a defined host cell including a target cell or tissue or a progenitor cell or tissue thereof, or be integrable with the genome of the defined host such that the cloned sequence is reproducible. Accordingly, the vector can be an autonomously replicating vector, i.e., a vector that exists as an extrachromosomal entity, the replication of which is independent of chromosomal replication, e.g., a linear or closed circular plasmid, an extrachromosomal element, a minichromosome, or an artificial chromosome. The vector can contain any means for assuring self-replication. Alternatively, the vector can be one which, when introduced into the host cell, is integrated into the genome and replicated together with the chromosome(s) into which it has been integrated. A vector system can comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. The vector can also include a selection marker such as an antibiotic resistance gene that can be used for selection of suitable transformants. Examples of such resistance genes are known to those of skill in the art.

### 2. Abbreviations

The following abbreviations are used throughout the application:

nts = nucleotides
aa = amino acid(s)
kb = kilobase(s) or kilobase pair(s)
kDa = kilodalton(s)
hr = hour
C. ° = degrees Celcius
mM = millimolar
μm = micrometer
g = gram
mg = milligram
μg = microgram
ng = nanogram
μl = microliter
cm = centimeter
% = percent
min = minute

### 3. Methods of Treating Dermal Injury

The present invention provides methods for treating a dermal injury, as disclosed in the Summary, including the administration to a subject of an effective amount of fetuin (AHSG) polypeptide or an agent from which a fetuin polypeptide is producible. In an illustrative embodiment, the dermal injury is a burn injury. Burn injuries fall into three categories of severity and the present methods are suitable for use in the treatment of burns in each category. Thus, in some embodiments, the burn injury is a first or second degree burn. In other embodiments, the burn injury is a third degree burn injury. In the case of third degree burns in which the epidermal and dermal layers are destroyed in whole or in part, treatment often involves the provision of cellular material, skin grafts or synthetic or combination dermal analogs (skin substitutes). In accordance with the present invention, fetuin polypeptides or agents from which fetuin polypeptides are producible are applied to or otherwise associated with a medical device or medical material such as cellular material, skin grafts, combination or synthetic substitute skin preparations. In other embodiments, the fetuin polypeptide or the agent from which the fetuin polypeptide is producible is applied to or otherwise associated with a medical device such as a suture, gauze or dressing.

Fetuin species homologs sharing more that about 60% amino acid sequence similarity have been identified in man, cattle, sheep, pig, goats, rabbits rats and mice. The human homolog is designated alpha-2-Heremans-Schmid (HS) glycoprotein (AHSG) and the mouse homolog is designated Countertrypsin. In accordance with the present invention, a fetuin polypeptide encompasses any naturally-occurring fetuin polypeptide from any animal species as well as their biologically active portions and variants or derivatives of these, as defined herein.

Fetuin polypeptides may be prepared by any suitable procedure known to those of skill in the art. For example, the polypeptides may be prepared by a procedure including the steps of: (a) preparing a construct comprising a polynucleotide sequence that encodes fetuin polypeptide and that is operably linked to a regulatory element; (b) introducing the construct into a host cell; (c) culturing the host cell to express the fetuin polypeptide; and (d) isolating the fetuin polypeptide from the host cell. In illustrative examples, the nucleotide sequence encodes at least a biologically active portion of the sequence set forth in any one of SEQ ID NO: 2, 5, 7, 9, 11, 13, 15, and 17, or a variant thereof. Positive and negative regulatory elements that modulate promoter activity of human fetuin (α2-HS-glycoprotein) are described for example in Banine F. et al., 2000, Eur. J. Biochem., 267:1214-1222. Recombinant fetuin polypeptides can be conveniently prepared using standard protocols as described for example in Sambrook, et al., (1989, supra), in particular Sections 16 and 17; Ausubel et al., (1994, supra), in particular Chapters 10 and 16; and Coligan et al., CURRENT PROTOCOLS IN PROTEIN SCIENCE (John Wiley & Sons, Inc. 1995-1997), in particular Chapters 1, 5 and 6. Alternatively, the fetuin polypeptides may be synthesized by chemical synthesis, e.g., using solution synthesis or solid phase synthesis as described, for example, in Chapter 9 of Atherton and Shephard (supra) and in Roberge et al. (1995, Science 269:202). The fetuin polypeptide may be produced by any convenient method such as by purifying the polypeptide from naturally-occurring reservoirs including blood or serum. Methods of purification include lectin (e.g. wheat germ agglutinin) affinity chromatography or separation. The identity and purity of derived fetuin is determined for example by SDS-polyacrylamide electrophoresis or chromatographically such as by high performance liquid chromatography (HPLC).

In other embodiments, the present invention provides a method for treating a dermal injury comprising debriding the dermal injury to remove devitalised tissue (eschar in the case of burn wounds). This step, inter alia facilitates delivery of the fetuin polypeptide when administration is local rather than systemic. In some embodiments, therefore, the method includes debriding the dermal injury prior to administration of fetuin polypeptide or an agent from which fetuin polypeptide is producible. In other embodiments, a debriding step is taken before, during and/or after administration of the fetuin polypeptide or fetuin producing agent.

The dermal injury may be debrided by any convenient means such as autolytically (using the subjects own enzymes and moisture), enzymatically (using selective (e.g. collagenase) or non-selective enzymes (e.g. papain and urea), biologically (using maggots), chemically (e.g. using hypochlorite), mechanically (e.g. hydrotherapy or adhesion to material such as a dressing) or surgically (using a sharp implement or laser).

The fetuin polypeptide of the present invention includes all biologically active naturally occurring forms of fetuin (AHSG) as well as biologically active portions thereof, and variants or derivatives of these. Biological activity as used herein refers to the ability of fetuin polypeptide to enhance healing of dermal injuries. Biologically active portions of fetuin polypeptide include parts of the amino acid sequence set out in any one of SEQ ID NO: 2, 5, 7, 9, 11, 13, 15, and 17. A biologically active portion of a full-length fetuin polypeptide may comprise, for example, at least about 5, 6, 7, 8, 9, 10, 11, 12, 13, 14, 15, 16, 17, 18, 19, 20, 21, 22, 23, 24, 25, 30, 40, 50, 60, 70, 80, 90, 100, 120 or 150, or even at least about 200, 220, 240, 260, 280, 300, 310, 320, 330, 340 or 350 contiguous amino acid residues, or almost up to the total number of amino acids present in a full-length fetuin polypeptide. Suitably, the portion is a “biologically-active portion” having no less than about 10%, 20%, 30%, 40% 50%, 60%, 70%, 80%, 90%, 99% of the activity of the full-length fetuin polypeptide from which it is derived. Suitable biologically active portions include soluble forms of the polypeptide without a leader or signal peptide.

Fetuin polypeptides includes “variant” polypeptides that are distinguished from a naturally-occurring fetuin polypeptide or from a biologically active portion thereof by the addition, deletion and/or substitution of at least one amino acid residue. Thus, variants include proteins derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Variant proteins encompassed by the present invention are biologically active, that is, they continue to possess the desired biological activity of the native protein (e.g., wound-treating activity). Such variants may result from, for example, genetic polymorphism or from human manipulation. Biologically active variants of a native fetuin polypeptide will have at least 40%, 50%, 60%, 70%, generally at least 75%, 80%, 85%, preferably about 90% to 95% or more, and more preferably about 98% or more sequence similarity or identity with the amino acid sequence for the native protein as determined by sequence alignment programs described elsewhere herein using default parameters. A biologically active variant of a fetuin polypeptide may differ from that polypeptide generally by as much 100, 50 or 20 amino acid residues or suitably by as few as 1-15 amino acid residues, as few as 1-10, such as 6-10, as few as 5, as few as 4, 3, 2, or even 1 amino acid residue.

A fetuin polypeptide may be altered in various ways including amino acid substitutions, deletions, truncations, and insertions. Methods for such manipulations are generally known in the art. For example, amino acid sequence variants of a fetuin polypeptide can be prepared by mutations in the DNA. Methods for mutagenesis and nucleotide sequence alterations are well known in the art. See, for example, Kunkel (1985, Proc. Natl. Acad. Sci. USA 82:488-492), Kunkel et al. (1987, Methods in Enzymol. 154:367-382), U.S. Pat. No. 4,873,192, Watson, J. D. et al. (“Molecular Biology of the Gene”, Fourth Edition, Benjamin/Cummings, Menlo Park, Calif., 1987) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.). Methods for screening gene products of combinatorial libraries made by point mutations or truncation, and for screening cDNA libraries for gene products having a selected property are known in the art. Such methods are adaptable for rapid screening of the gene libraries generated by combinatorial mutagenesis of fetuin polypeptides. Recursive ensemble mutagenesis (REM), a technique which enhances the frequency of functional mutants in the libraries, can be used in combination with the screening assays to identify fetuin polypeptide variants (Arkin and Yourvan (1992) Proc. Natl. Acad. Sci. USA 89:7811-7815; Delgrave et al. (1993) Protein Engineering 6:327-331). Conservative substitutions, such as exchanging one amino acid with another having similar properties, may be desirable as discussed in more detail below.

Variant fetuin polypeptides may contain conservative amino acid substitutions at various locations along their sequence, as compared to the parent fetuin amino acid sequence. A “conservative amino acid substitution” is one in which the amino acid residue is replaced with an amino acid residue having a similar side chain. Families of amino acid residues having similar side chains have been defined in the art, which can be generally sub-classified as follows:

Acidic: The residue has a negative charge due to loss of H ion at physiological pH and the residue is attracted by aqueous solution so as to seek the surface positions in the conformation of a peptide in which it is contained when the peptide is in aqueous medium at physiological pH. Amino acids having an acidic side chain include glutamic acid and aspartic acid.

Basic: The residue has a positive charge due to association with H ion at physiological pH or within one or two pH units thereof (e.g., histidine) and the residue is attracted by aqueous solution so as to seek the surface positions in the conformation of a peptide in which it is contained when the peptide is in aqueous medium at physiological pH. Amino acids having a basic side chain include arginine, lysine and histidine.

Charged: The residues are charged at physiological pH and, therefore, include amino acids having acidic or basic side chains (i.e., glutamic acid, aspartic acid, arginine, lysine and histidine).

Hydrophobic: The residues are not charged at physiological pH and the residue is repelled by aqueous solution so as to seek the inner positions in the conformation of a peptide in which it is contained when the peptide is in aqueous medium. Amino acids having a hydrophobic side chain include tyrosine, valine, isoleucine, leucine, methionine, phenylalanine and tryptophan.

Neutral/polar: The residues are not charged at physiological pH, but the residue is not sufficiently repelled by aqueous solutions so that it would seek inner positions in the conformation of a peptide in which it is contained when the peptide is in aqueous medium. Amino acids having a neutral/polar side chain include asparagine, glutamine, cysteine, histidine, serine and threonine.

This description also characterizes certain amino acids as “small” since their side chains are not sufficiently large, even if polar groups are lacking, to confer hydrophobicity. With the exception of proline, “small” amino acids are those with four carbons or less when at least one polar group is on the side chain and three carbons or less when not. Amino acids having a small side chain include glycine, serine, alanine and threonine. The gene-encoded secondary amino acid proline is a special case due to its known effects on the secondary conformation of peptide chains. The structure of proline differs from all the other naturally-occurring amino acids in that its side chain is bonded to the nitrogen of the α-amino group, as well as the α-carbon. Several amino acid similarity matrices (e.g., PAM120 matrix and PAM250 matrix as disclosed for example by Dayhoff et al. (1978), A model of evolutionary change in proteins. Matrices for determining distance relationships In M. O. Dayhoff, (ed.), Atlas of protein sequence and structure, Vol. 5, pp. 345-358, National Biomedical Research Foundation, Washington D.C.; and by Gonnet et al., 1992, Science 256(5062): 144301445), however, include proline in the same group as glycine, serine, alanine and threonine. Accordingly, for the purposes of the present invention, proline is classified as a “small” amino acid.

The degree of attraction or repulsion required for classification as polar or nonpolar is arbitrary and, therefore, amino acids specifically contemplated by the invention have been classified as one or the other. Most amino acids not specifically named can be classified on the basis of known behavior.

Amino acid residues can be further sub-classified as cyclic or noncyclic, and aromatic or nonaromatic, self-explanatory classifications with respect to the side-chain substituent groups of the residues, and as small or large. The residue is considered small if it contains a total of four carbon atoms or less, inclusive of the carboxyl carbon, provided an additional polar substituent is present; three or less if not. Small residues are, of course, always nonaromatic. Dependent on their structural properties, amino acid residues may fall in two or more classes. For the naturally-occurring protein amino acids, sub-classification according to this scheme is presented in the Table A.

TABLE A
AMINO ACID SUB-CLASSIFICATION

| Sub-classes | Amino acids |
|---|---|
| Acidic | Aspartic acid, Glutamic acid |
| Basic | Noncyclic: Arginine, Lysine; Cyclic: Histidine |
| Charged | Aspartic acid, Glutamic acid, Arginine, Lysine, |
| | Histidine |
| Small | Glycine, Serine, Alanine, Threonine, Proline |
| Polar/neutral | Asparagine, Histidine, Glutamine, Cysteine, Serine, |
| | Threonine |
| Polar/large | Asparagine, Glutamine |
| Hydrophobic | Tyrosine, Valine, Isoleucine, Leucine, Methionine, |
| | Phenylalanine, Tryptophan |
| Aromatic | Tryptophan, Tyrosine, Phenylalanine |
| Residues that | Glycine and Proline |
| influence chain | |
| orientation | |

Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting variant polypeptide. Whether an amino acid change results in a functional fetuin polypeptide can readily be determined by assaying its activity. Conservative substitutions are shown in Table B below under the heading of exemplary substitutions. More preferred substitutions are shown under the heading of preferred substitutions. Amino acid substitutions falling within the scope of the invention, are, in general, accomplished by selecting substitutions that do not differ significantly in their effect on maintaining (a) the structure of the peptide backbone in the area of the substitution, (b) the charge or hydrophobicity of the molecule at the target site, or (c) the bulk of the side chain. After the substitutions are introduced, the variants are screened for biological activity.

TABLE B
EXEMPLARY AND PREFERRED
AMINO ACID SUBSTITUTIONS

| ORIGINAL | | PREFERRED |
|---|---|---|
| RESIDUE | EXEMPLARY SUBSTITUTIONS | SUBSTITUTIONS |
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn, His, Lys, | Asn |
| Glu | Asp, Lys | Asp |
| Gly | Pro | Pro |
| His | Asn, Gln, Lys, Arg | Arg |
| Ile | Leu, Val, Met, Ala, Phe, Norleu | Leu |
| Leu | Norleu, Ile, Val, Met, Ala, Phe | Ile |
| Lys | Arg, Gln, Asn | Arg |
| Met | Leu, Ile, Phe | Leu |
| Phe | Leu, Val, Ile, Ala | Leu |
| Pro | Gly | Gly |
| Ser | Thr | Thr |
| Thr | Ser | Ser |
| Trp | Tyr | Tyr |
| Tyr | Trp, Phe, Thr, Ser | Phe |
| Val | Ile, Leu, Met, Phe, Ala, Nor, Leu | Leu |

Alternatively, similar amino acids for making conservative substitutions can be grouped into three categories based on the identity of the side chains. The first group includes glutamic acid, aspartic acid, arginine, lysine, histidine, which all have charged side chains; the second group includes glycine, serine, threonine, cysteine, tyrosine, glutamine, asparagine; and the third group includes leucine, isoleucine, valine, alanine, proline, phenylalanine, tryptophan, methionine, as described in Zubay, G., Biochemistry, third edition, Wm.C. Brown Publishers (1993).

Thus, a predicted non-essential amino acid residue in a fetuin polypeptide is typically replaced with another amino acid residue from the same side chain family. Alternatively, mutations can be introduced randomly along all or part of a fetuin polynucleotide coding sequence, such as by saturation mutagenesis, and the resultant mutants can be screened for an activity of the parent polypeptide to identify mutants which retain that activity. Following mutagenesis of the coding sequences, the encoded peptide can be expressed recombinantly and the activity of the peptide can be determined.

Accordingly, the present invention also contemplates as fetuin polypeptides, variants of the naturally-occurring fetuin polypeptide sequences or their biologically-active fragments, wherein the variants are distinguished from the naturally-occurring sequence by the addition, deletion, or substitution of one or more amino acid residues. In general, variants will display at least about 30, 40, 50, 55, 60, 65, 70, 75, 80, 85, 90, 91, 92, 93, 94, 95, 96, 97, 98, 99% similarity to a parent fetuin polypeptide sequence as, for example, set forth in any one of SEQ ID NO: 2, 5, 7, 9, 11, 13, 15, and 17. Desirably, variants will have at least 30, 40, 50, 55, 60, 65, 70, 75, 80, 85, 90, 91, 92, 93, 94, 95, 96, 97, 98, 99% sequence identity to a parent fetuin polypeptide sequence as, for example, set forth in any one of SEQ ID NO: 2, 5, 7, 9, 11, 13, 15, and 17. Moreover, sequences differing from the native or parent sequences by the addition, deletion, or substitution of 1, 2, 3, 4, 5, 6, 7, 8, 9, 10, 11, 12, 13, 14, 15, 16, 17, 18, 19, 20, 30, 40, 50, 60, 70, 80, 90, 100 or more amino acids but which retain the properties of the parent fetuin polypeptide are contemplated. Fetuin polypeptides also include polypeptides that are encoded by polynucleotides that hybridize under stringency conditions as defined herein, especially high stringency conditions, to fetuin polynucleotide sequences, or the non-coding strand thereof. Illustrative fetuin polynucleotide sequences are set forth in SEQ ID NO: 1, 3, 4, 6, 8, 10, 12, 14, and 16.

In some embodiments, variant polypeptides differ from a naturally-occurring fetuin sequence by at least one but by less than 50, 40, 30, 20, 15, 10, 8, 6, 5, 4, 3 or 2 amino acid residue(s). In another, variant polypeptides differ from the corresponding sequence in any one of SEQ ID NO: 2 by at least 1% but less than 20%, 15%, 10% or 5% of the residues. (If this comparison requires alignment the sequences should be aligned for maximum similarity. “Looped” out sequences from deletions or insertions, or mismatches, are considered differences.) The differences are, suitably, differences or changes at a non-essential residue or a conservative substitution. Naturally-occurring fetuin polypeptides contain a significant number of structural characteristics in common with each other as for example depicted in FIG. 18. This alignment shows positions that are amenable to conservative substitution and others that accommodate non-conservative substitutions.

A “non-essential” amino acid residue is a residue that can be altered from the wild-type sequence of an embodiment polypeptide without abolishing or substantially altering one or more of its activities. Suitably, the alteration does not substantially alter one of these activities, for example, the activity is at least 20%, 40%, 60%, 70% or 80% of wild-type. An “essential” amino acid residue is a residue that, when altered from the wild-type sequence of a fetuin polypeptide, results in abolition of an activity of the parent molecule such that less than 20% of the wild-type activity is present. For example, amino acid residues that are absolutely conserved between the fetuin polypeptides of human, chimpanzee, mouse, rat, cattle, sheep, pig and guinea pig, as shown in FIG. 18, may be unamenable to alteration.

In other embodiments, a variant polypeptide includes an amino acid sequence having at least about 50%, 55%, 60%, 65%, 70%, 75%, 80%, 85%, 90%, 91%, 92%, 93%, 94% 95%, 96%, 97%, 98% or more similarity to a corresponding sequence of a fetuin polypeptide as, for example, set forth in any one of SEQ ID NO: 2, 5, 7, 9, 11, 13, 15, and 17, and has the activity of a fetuin polypeptide.

Another useful group of compounds are functional derivatives, analogs and mimics (mimetics) of fetuin. In accordance with the present invention, these molecules retain the ability to enhance dermal healing and may also possess additional characteristics which improve their efficacy, such as exhibiting a longer half life in vivo or alternatively which are, for example, readily synthesized or readily taken up by skin cells. A peptide mimetic or mimic has some chemical similarity to the parent molecule e.g., fetuin, but agonizes its activity. A peptide mimic may be a peptide-containing molecule which mimics elements of protein secondary structure (as described for example in Johnson et al “Peptide Turn Mimetics” in Biotechnology and Pharmacy, Pezzuto et al, Eds., Chapman and Hall, New York, 1993) Peptide or non-peptide mimetics may be useful, for example, in antagonizing TGF-β activity in the skin. Non-peptide “small molecules” are often preferred for many in vivo pharmaceutical applications and accordingly mimetics may be designed for pharmaceutical use. Mimetic design, synthesis and testing is generally used to avoid randomly screening large numbers of molecules for a particular property, particularly where a lead compound has already been identified. As a first step, residues critical for enhancing healing are identified and this framework used as a pharmacophore. The structure may then be modeled using computational and other analyses. Alternatively, the three dimensional structure of the agent may be known in which case further agents may be designed along the same lines.

The goal of rational drug design is to produce structural analogs of biologically active polypeptides of interest or of small molecules with which they interact (e.g. agonists, antagonists, inhibitors or enhancers) in order to fashion drugs which are, for example, more active or stable forms of the polypeptide, or which, e.g. enhance or interfere with the function of a polypeptide in vivo. See, e.g. Hodgson (1991, Bio/Technology, 9:19-21). In one approach, one first determines the three-dimensional structure of a protein of interest by x-ray crystallography, by computer modeling or most typically, by a combination of approaches. Useful information regarding the structure of a polypeptide may also be gained by modeling based on the structure of homologous proteins. An example of rational drug design is the development of HIV protease inhibitors (Erickson et al., 1990, Science, 249:527-533). In addition, target molecules may be analyzed by an alanine scan (Wells, 1991, Methods Enzymol, 202:2699-2705). In this technique, an amino acid residue is replaced by Ala and its effect on the peptide's activity is determined. Each of the amino acid residues of the peptide is analyzed in this manner to determine the important regions of the peptide.

It is also possible to isolate a target-specific antibody, selected by a functional assay and then to solve its crystal structure. In principle, this approach yields a pharmacophore upon which subsequent drug design can be based. It is possible to bypass protein crystallography altogether by generating anti-idiotypic antibodies (anti-ids) to a functional, pharmacologically active antibody. As a mirror image of a mirror image, the binding site of the anti-ids would be expected to be an analog of the original receptor. The anti-id could then be used to identify and isolate peptides from banks of chemically or biologically produced banks of peptides. Selected peptides would then act as the pharmacophore.

Agents capable of producing fetuin polypeptide conveniently include without limitation cells from which fetuin polypeptide is produced or polynucleotides encoding fetuin polypeptides.

Analogs contemplated herein include but are not limited to modification to side chains, incorporating of unnatural amino acids and/or their derivatives during peptide, polypeptide or protein synthesis and the use of crosslinkers and other methods which impose conformational constraints on the proteinaceous molecule or their analogs. This term also does not exclude modifications of the polypeptide, for example, glycosylations, aceylations, phosphorylations and the like. Included within the definition are, for example, polypeptides containing one or more analogs of an amino acid or polypeptides with substituted linkages. Such polypeptides may need to be able to enter the cell.

Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2,4,6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivitization, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carbethoxylation with diethylpyrocarbonate.

Examples of incorporating unnatural amino acids and derivatives during peptide synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids.

Fetuin is a glycoprotein and variants of the carbohydrate residues attached to fetuin are also contemplated. Sugar chains may for example be modified enzymatically and then tested for binding to known substrates such as lectins or growth factors or cytokines. Sialidase treated fetuin-liposome conjugates are described for example in Yamazaki et al., 1997, Fetuin-liposome conjugates and immobilized lectins as a model system for studying multivalent carbohydrate-lectin interactions. In: Lectins, Biology, Biochemistry, Clinical Biochemistry, van Driessche et al., Eds., Vol. 12 including Proceedings from the 17^{th} International Lectin Meeting in Wurzburg, Denmark, TEXTOP.

In some embodiments, the agent is an autologous cell derived from the subject to be treated or a syngeneic cell. In some embodiments, the cell is genetically modified in order to secrete a fetuin polypeptide. Other cells, such as monocytes or macrophages secrete fetuin polypeptide naturally. In other embodiments, the cell is a genetically modified dermal cell capable of producing fetuin polypeptide. In further embodiments, the dermal cell is an epidermal cell selected from one or more epidermal cell types such as keratinocytes, melanocytes and fibroblasts. In specific embodiments, the epidermal cell is a keratinocyte. In still further embodiments, the cell is a stem cell or progenitor cell for a dermal cell. In an illustrative example of this type, the stem cell is an epidermal cell progenitor cell. In illustrative example, the stem cell is a keratinocyte progenitor cell. Genetically modified stem cells are conveniently used in order to treat dermal injuries and enhance healing and to reduce scar formation or contracture.

Recombinant methods for producing genetically modified cells from which fetuin polypeptide is producible are routine in the art. Essentially, a polynucleotide encoding a fetuin polypeptide is engineered within an expression construct or shuttle vector and operably linked to a regulatory element (e.g. a promoter) that is operable in the cell in which it is desired to express the polynucleotide. The promoter may be inducible or constitutive, and, optionally, tissue-specific. The promoter may be, for example, viral or mammalian in origin. In some embodiments, a nucleic acid construct is used in which the promoter-polynucleotide cassette (and any other desired sequences) is flanked by regions that promote homologous recombination at a desired site within the genome of a subject, thus providing for intra-chromosomal expression of the polynucleotide. See e.g., Koller and Smithies, 1989. Proc Natl Acad Sci USA 86: 8932-8935. In other embodiments, the nucleic acid construct that is delivered remains episomal and induces an endogenous and otherwise silent gene. Generally, a selective marker gene such as an antibiotic resistance marker gene is employed to facilitate selection of appropriately modified cells. In some embodiments, the polynucleotide (cDNA) is selected (amplified) or modified by removal of sequences encoding signal sequences to facilitate secretion of a soluble or mature fetuin polypeptide. Mammalian expression vectors capable of expression in mammalian epidermal cells are, for example, routinely available. Construction of recombinant DNAs comprising fetuin polynucleotides and a mammalian vector capable of expressing inserted DNAs in cultured human or animal cells, can be carried out by standard gene expression technology using methods well known in the art for expression of such a relatively simple polypeptide. The polynucleotides encoding several different fetuin polypeptides have been cloned and expressed in a range of eukaryotic and prokaryotic expression systems and are commercially available. Promoters for selective expression in a range of dermal cells have also been identified and documented.

A number of viruses have been used as gene transfer vectors or as the basis for preparing gene transfer vectors, including papovaviruses, adenovirus, vaccinia virus, adeno-associated virus, herpesviruses including HSV and EBV, lentiviruses, Sindbis and Semliki Forest virus and retroviruses of avian and murine origin.

Viral and non-viral methods of polynucleotide delivery are available. Various viral vectors are routinely used to transform a range of different cell types with adequate efficiency.

Exemplary nucleic acid constructs that are operable in mammalian cells include retro-, adeno- or adeno-associated or lentiviral vectors. In one illustrative embodiment, retroviruses provide a convenient and effective platform for gene delivery systems. A nucleotide sequence for which at least one control element of the present invention is expressible can be inserted into a vector and packaged in retroviral particles using techniques known in the art. The recombinant virus can then be isolated and delivered to a subject. Several illustrative retroviral systems have been described examples of which include: U.S. Pat. No. 5,219,740; Miller and Rosman, 1989, Bio Techniques 7: 980-990; Miller, A. D., 1990, Human Gene Therapy 1: 5-14; Scarpa et al., 1991, Virology 180: 849-852; Burns et al., 1993, Proc. Natl. Acad. Sci. USA 90: 8033-8037; and Boris-Lawrie and Temin, 1993, Cur. Opin. Genet. Develop. 3: 102-109).

In addition, several illustrative adenovirus-based systems have been described. Unlike retroviruses which integrate into the host genome, adenoviruses persist extrachromosomally thus minimising the risks associated with insertional mutagenesis (see, e.g., Haj-Ahmad and Graham, 1986, J. Virol. 57: 267-274; Bett et al., 1993, J. Virol. 67: 5911-5921; Mittereder et al., 1994, Human Gene Therapy 5: 717-729; Seth et al., 1994, J. Virol. 68: 933-940; Barr et al., 1994, Gene Therapy 1: 51-58; Berkner, K. L., 1988, Bio Techniques 6: 616-629; and Rich et al., 1993, Human Gene Therapy 4: 461-476).

Various adeno-associated virus (AAV) vector systems have also been developed for polynucleotide delivery. AAV vectors can be readily constructed using techniques well known in the art. See, e.g., U.S. Pat. Nos. 5,173,414 and 5,139,941; International Publication Nos. WO 92/01070 and WO 93/03769; Lebkowski et al., 1988, Molec. Cell. Biol. 8: 3988-3996; Vincent et al., 1990, Vaccines 90, Cold Spring Harbor Laboratory Press; Carter, B. J., 1992, Current Opinion in Biotechnology 3: 533-539; Muzyczka, N., 1992, Current Topics in Microbiol. and Immunol. 158: 97-129; Kotin, R. M., 1994, Human Gene Therapy 5: 793-801; Shelling and Smith, 1994, Gene Therapy 1: 165-169; and Zhou et al., 1994, J. Exp. Med. 179: 1867-1875.

Alternatively, avipoxviruses, such as the fowlpox and canarypox viruses, can also be used to deliver the coding sequences of interest. The use of an Avipox vector is particularly desirable in human and other mammalian species since members of the Avipox genus can only productively replicate in susceptible avian species and therefore are not infective in mammalian cells. Methods for producing recombinant Avipoxviruses are known in the art and employ genetic recombination, as described above with respect to the production of vaccinia viruses. See, e.g., WO 91/12882; WO 89/03429; and WO 92/03545.

Any of a number of alphavirus vectors can also be used for delivery of polynucleotide compositions of the present invention, such as those vectors described in U.S. Pat. Nos. 5,843,723; 6,015,686; 6,008,035 and 6,015,694. Certain vectors based on Venezuelan Equine Encephalitis (VEE) can also be used, illustrative examples of which can be found in U.S. Pat. Nos. 5,505,947 and 5,643,576.

Moreover, molecular conjugate vectors, such as the adenovirus chimeric vectors described in Michael et al., J. Biol. Chem. 268:6866-69, 1993; and Wagner et al., Proc. Natl. Acad. Sci. USA 89:6099-6103, 1992, can also be used for fetuin polynucleotide.

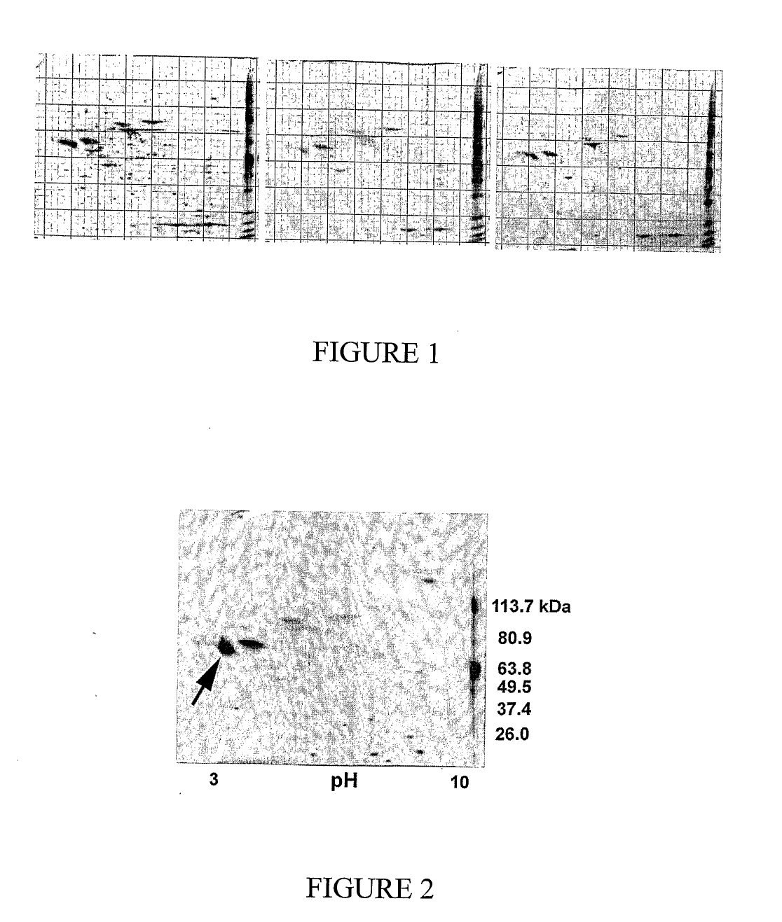

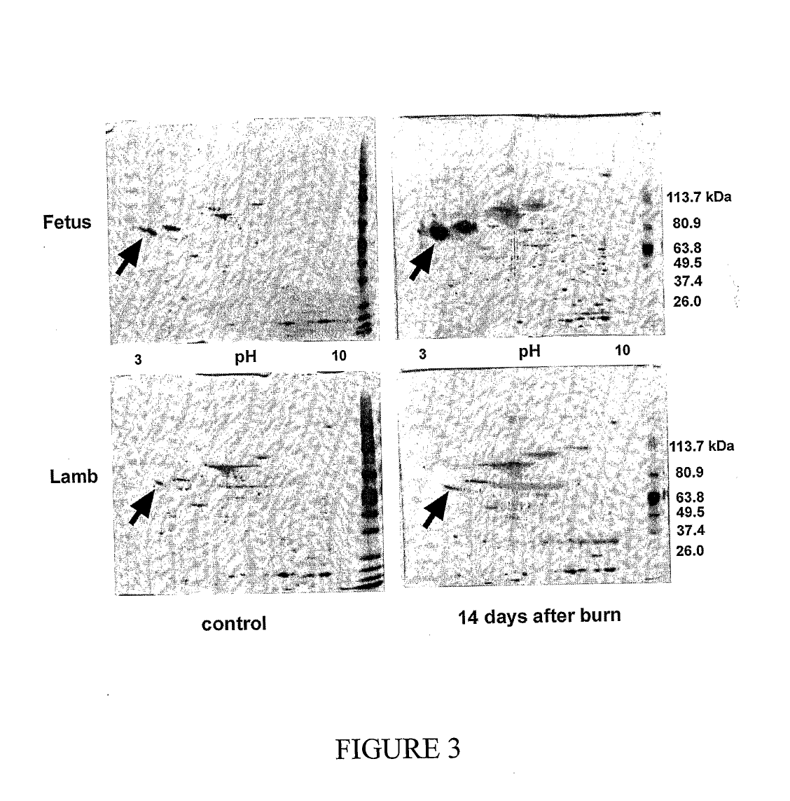

In other illustrative embodiments, lentiviral vectors are employed to deliver the fetuin polynucleotide into selected cells or tissues. Typically, these vectors comprise a 5′ lentiviral LTR, a tRNA binding site, a packaging signal, a promoter operably linked to one or more genes of interest, an origin of second strand DNA synthesis and a 3′ lentiviral LTR, wherein the lentiviral vector contains a nuclear transport element. The nuclear transport element may be located either upstream (5′) or downstream (3′) of a coding sequence of interest (for example, a synthetic Gag or Env expression cassette of the present invention). A wide variety of lentiviruses may be utilized within the context of the present invention, including for example, lentiviruses selected from the group consisting of HIV, HIV-1, HIV-2, FIV, BIV, EIAV, MVV, CAEV, and SIV. Illustrative examples of lentiviral vectors are described in PCT Publication Nos. WO 00/66759, WO 00/00600, WO 99/24465, WO 98/51810, WO 99/51754, WO 99/31251, WO 99/30742, and WO 99/15641. Desirably, a third generation SIN lentivirus is used. Commercial suppliers of third generation SIN (self-inactivating) lentiviruses include Invitrogen (ViraPower Lentiviral Expression System). Detailed methods for construction, transfection, harvesting, and use of lentiviral vectors are given, for example, in the Invitrogen technical manual “ViraPower Lentiviral Expression System version B 050102 25-0501”, available at http://www.invitrogen.com/Content/Tech-Online/molecular_biology/manuals_p-ps/virapower_lentiviral_system_man.pdf. Lentiviral vectors have emerged as an efficient method for gene transfer. Improvements in biosafety characteristics have made these vectors suitable for use at biosafety level 2 (BL2). A number of safety features are incorporated into third generation SIN (self-inactivating) vectors. Deletion of the viral 3′ LTR U3 region results in a provirus that is unable to transcribe a full length viral RNA. In addition, a number of essential genes are provided in trans, yielding a viral stock that is capable of but a single round of infection and integration. Lentiviral vectors have several advantages, including: 1) pseudotyping of the vector using amphotropic envelope proteins allows them to infect virtually any cell type; 2) gene delivery to quiescent, post mitotic, differentiated cells, including neurons, has been demonstrated; 3) their low cellular toxicity is unique among transgene delivery systems; 4) viral integration into the genome permits long term transgene expression; 5) their packaging capacity (6-14 kb) is much larger than other retroviral, or adeno-associated viral vectors. In a recent demonstration of the capabilities of this system, lentiviral vectors expressing GFP were used to infect murine stem cells resulting in live progeny, germline transmission, and promoter-, and tissue-specific expression of the reporter (Ailles, L. E. and Naldini, L., HIV-1-Derived Lentiviral Vectors. In: Trono, D. (Ed.), Lentiviral Vectors, Springer-Verlag, Berlin, Heidelberg, New York, 2002, pp. 31-52). An example of the current generation vectors is outlined in FIG. 2 of a review by Lois et al. (Lois, C., Hong, E. J., Pease, S., Brown, E. J., and Baltimore, D., Germline transmission and tissue-specific expression of transgenes delivered by lentiviral vectors, Science, 295 (2002) 868-872).

In certain embodiments, a polynucleotide may be integrated into the genome of a target cell, especially a target dermal cell. This integration may be in the specific location and orientation via homologous recombination (gene replacement) or it may be integrated in a random, non-specific location (gene augmentation). In yet further embodiments, the polynucleotide may be stably maintained in the cell as a separate, episomal segment of DNA. Such polynucleotide segments or “episomes” encode sequences sufficient to permit maintenance and replication independent of or in synchronization with the host cell cycle. The manner in which the expression construct is delivered to a cell and where in the cell the polynucleotide remains is dependent on the type of expression construct employed.

Vector and host cell combinations have been used successfully to express several other similar recombinant polypeptides, including high levels of Platelet-Derived Growth Factor (PDGF) A and B chains (Sakai, R. K., Scharf, S., Faloona, F., Mullis, K. B., Norn, G. T., Erlich, H. A. and Arnheim, N. (1985) Science 230, 1350-1354). Accordingly, it will be recognized by those skilled in the art that secretion of recombinant fetuin polypeptide from cells of dermal origin can be achieved in this manner.

Production of polypeptides in a target cell, e.g. in a viral vector has been previously described together with a cell based delivery system such as described in U.S. Pat. No. 5,550,050 and International Patent Publication Nos. WO 92/19195, WO 94/25503, WO 95/01203, WO 95/05452, WO 96/02286, WO 96/02646, WO 96/40871, WO 96/40959 and WO 97/12635. The vector could be targeted to the target skin cells or expression of expression products could be limited to specific cells, stages of development or cell cycle stages. The cell based delivery system is designed to be implanted in a patient's body at the burn site and to there produce fetuin polypeptide. Alternatively, the polypeptide could be produced in a precursor form for conversion to the active form by an activating agent produced in, or targeted to, the dermal injury be treated. See, for example, European Patent Application No. 0 425 731A and International Patent Publication No. WO 90/07936.

Non-viral methods have also been successfully used to transform mammalian cells with high efficiency. Illustrative non-viral methods include without limitation, gene gun particle bombardment, electrospray, co-precipitation, electroporation, pressure perfusion, ultrasound, focused lasers and magnetofection using magnetic nanoparticles. Mechanical techniques, for example, include microinjection, membrane fusion-mediated transfer via liposomes and direct DNA uptake based approaches. These methods are described in U.S. Pat. Nos. 4,945,050, 5,120,657 and 6,093,557.

Viral-mediated gene transfer can be combined with direct in vivo gene transfer using liposome delivery, allowing one to direct the viral vectors to particular cells. Alternatively, the retroviral vector producer cell line can be injected into particular tissue. Injection of producer cells would then provide a continuous source of vector particles. In an approach which combines biological and physical gene transfer methods, plasmid DNA of any size is combined with a polylysine-conjugated antibody specific to the adenovirus hexon protein and the resulting complex is bound to an adenovirus vector. The trimolecular complex is then used to infect cells. The adenovirus vector permits efficient binding, internalization and degradation of the endosome before the coupled DNA is damaged. For other techniques for the delivery of adenovirus based vectors, see U.S. Pat. No. 5,691,198.

In some embodiments, the fetuin polynucleotide comprises a nucleotide sequence that encodes wholly or partially the amino acid sequence set forth in any one of SEQ ID NO: 2, 5, 7, 9, 11, 13, 15, and 17 or a nucleotide sequence that shares at least 60% (and at least 61% to at least 99% and all integer percentages in between) sequence identity with the sequence set forth in any one of SEQ ID NO: 2, 5, 7, 9, 11, 13, 15, and 17. In some embodiments, the fetuin polynucleotide comprises all or part of the nucleotide sequence set forth in any one of SEQ ID NO: 1, 3, 4, 6, 8, 10, 12, 14 and 16, or a nucleotide sequence that shares at least 60% (and at least 61% to at least 99% and all integer percentages in between) sequence identity with the sequence set forth in any one of SEQ ID NO: 1, 3, 4, 6, 8, 10, 12, 14 and 16, or a sequence that hybridizes to any one of SEQ ID NO: 1, 3, 4, 6, 8, 10, 12, 14 and 16 or to a complementary form thereof under at least medium stringency conditions.

In another aspect, the present invention provides a composition for use in the treatment of a dermal injury comprising a fetuin polypeptide or an agent from which a fetuin polypeptide is producible. In some embodiments the composition comprises a pharmaceutically acceptable carrier, diluent and/or excipient. In some embodiments, the composition further comprises or is co-administered with an ancillary agent which enhances would healing. Illustrative wound-healing agents include: cytokines factors (see for example Werner et al., 2003, Physio Rev 83:835-870, 2003); keratinocyte growth factors (KGFs, see for example, Antioniades et al., 1991, Proc. Natl. Acad. Sci. USA 88:565; Beer et al., 2000, J. Investig. Dermatol. Symp. Proc. 5:34; and U.S. Pat. Pub. No. 20040224387); platelet-derived growth factors (PDGFs, see for example, Antioniades et al., 1991, Proc. Natl. Acad. Sci. USA 88:565; and Staiano-Coico. et al., 1993, J. Exp. Med. 178:865-878); fibroblast growth factors (FGFs, see for example, Golden et al., 1991, J. Clin. Invest. 87:406; Mellin et al., 1995, J. Invest. Dermatol. 104:850-855); epidermal growth factors (EGFs, see for example, Whitby. and Ferguson 1991, Dev. Biol. 147:207); neu differentiation factor (see for example, Danilenko et al., 1995, J. Clin. Invest. 95; 842-851); insulin-like growth factors (IGFs, see for example, Cromack et al., 1987, J. Surg. Res. 42:622); and agents that modulate the level or functional activity of transforming growth factors (TGFs).

In certain embodiments, the wound-healing agent modulates the level or activity of one or more members of the TGF-β family. In specific examples of this type, the wound-healing agent is a non-fibrotic TGF-β molecule, such as TGF-β3.

The wound-healing agent may agonize or antagonize the activity of one or more TGF-β family members. In some embodiments, the wound-healing agent is selected from a TGF-β binding protein, a TGF-β receptor binding protein, an antibody, an antigen-binding fragment or analog thereof or a soluble receptor or part thereof which inhibits TGF-β activity. In an illustrative embodiment TGF-β-binding protein is Latency Associated Protein (LAP). In other embodiments, the antagonist is a chemical molecule such as for example, those described in US Publication Nos. 2004/0146509, 2004/0180905, 2004/0176390, 2004/0157861, 2004/0147574, 2004/0138188, 2004/0116474, 2004/0116473, 2004/0110798, 2004/0110797, 2004/0106608, 2004/0106604 and 2004/0039198 incorporated by reference herein in their entirety. Other antagonist include ursolic acid, SD-208, and SB-431542. Polypeptide inhibitors include cystatin C, r150 (a TGFB receptor accessory protein) Noggin, Chordin and Chordin-like proteins, Follistatin-related protein, Dan, Cerberus, Gremlin, Sclerostin (SOST) and Decorin.

Other ancillary wound-healing agents include collagenase/growth factor compositions, as described for example in U.S. Pat. No. 5,718,897; enzymes that degrade glycosaminoglycans such as heparin or chondroitin sulfate in various combinations, as described for example in U.S. Pat. No. 5,997,863; agents that activate the transglutaminase-1 gene, as described for example by Inada et al. (2000, Am. J. Pathol. 157:1875); viral vectors that deliver the gene for growth factor inducible element named “FiRE” into wound margin keratinocytes, as described for example by Jaakkola et al. (2000, Gene Ther. 7:1640,); amphipathic peptides that stimulate fibroblast and keratinocyte growth in vivo, as described for example in U.S. Pat. No. 6,001,805 and U.S. Pat. No. 6,191,110.

As discussed in relation the methods of the present invention, the subject composition, in some embodiments, comprises a cellular agent. In an illustrative example of this type the cell is a genetically modified syngeneic cell that produces fetuin polypeptide. In other embodiments, the cell is a viral cell capable of transforming dermal cells and causing them to produce fetuin polypeptide. In other embodiments, the cell naturally produces fetuin polypeptide such as a cell of the monocyte/macrophage lineage. Suitably, the administered cell is a dermal cell or stem cell capable of forming dermal cells. In some embodiments, the dermal cells are selected from one or more types of epidermal cell such as keratinocytes, melanocytes and fibroblasts, or their progenitors.

In another aspect, the present invention provides a use of a fetuin polypeptide or an agent from which a fetuin polypeptide is producible in the manufacture of a medicament for the treatment of a dermal injury. In some embodiments, the dermal injury is a burn injury, such as a thermal injury.

In some embodiments, the medicament is suitable for local or systemic administration by any route, such as without limitation by patch, cellular transfer, implant, orally, intravenously, intravesicaly, intracerebrally, intradermally, intramuscularly, intraperitonealy, intrathecally, subcutaneously, sublingually, rectally, vaginally, intraocularly, nasally, respiratorialy, nasopharyngealy, subcutaneously, cutaneously, topically and transdermally. Preferrably, the medicament is suitable for topical, transdermal, intradermal or cutaneous or subcutaneous administration at or near to the site of dermal injury. In some embodiments, the medicament is a film, gel, aerosol, powder, foam, colloid, liquid, solid or suspension. Film forming compositions for topical use and delivery of active agents are described for example in U.S. Pat. No. 6,797,262. Intradermal delivery of polypeptides is described in particular in published US Patent Application No. 20040073160.

As described hereinbefore, in some embodiments of the invention the agent is a cell which secretes a fetuin polypeptide. In some embodiments, the cells are dermal cells or stem cells capable of forming dermal cells. In other embodiments, the dermal cells are selected from one or more epidermal cells such as keratinocytes, melanocytes and fibroblasts.

In some embodiments, the polypeptide or agents are applied to, attached to or otherwise associated with a medical or other device, tissue or composition. In one embodiment, for example, the polypeptide, agent or composition is associated with a dressing. In another embodiment, they are associated with a suture, graft, substitute skin or other composition which is applied to the dermal or burn injury. In other embodiments, administration or delivery is by topical application of a gel, powder, film, aerosol, foam, colloid, cyanoacrylate polymer sealant, patch, emulsion, liquid or suspension effective to deliver the active agents to the burn site, wound bed and/or surrounding tissue. The agents and compositions can be formulated into a wide variety of carriers. For example, the active agents are formulated together with carriers such as microparticles, gels, bioactive foams, synthetic skin preparations, liquids and creams. Microparticles may for example be microspheres, microcapsules, liposomes and the like adapted for slow release of agents over time. Such particles are conveniently biodegradable or biocompatible.

In addition bioadhesive molecules such as bioadhesive peptides or polymers are conveniently used to bind polypeptides, cells, polynucleotides etc of the present invention to the dermal injury site and enhance their effectiveness. Film forming compositions and bioadhesive polymers are described in U.S. Pat. No. 6,103,266.

An emulsion is a composition comprising more than one phase where at least one of the phases consist of finely divided phase domains (such as, for example, particles or droplets) distributed throughout a continuous phase domain. An emulsion is formed, for example, when two immiscible liquids such as oil and water are sufficiently well mixed. The finely divided domains are generally referred to as dispersed or discontinuous phase domains. The dispersion may be further defined in terms of the size of the dispersed domains. Micro emulsions are particularly useful emulsions due to their thermodynamic stability and optical properties. A micro emulsion comprises dispersed domains having a diameter in the order of 10⁻⁶ M. A micro emulsion includes, for example a strict water in oil (or oil in water) micro emulsion, a bicontinuous monophase, a micellar solution or a swollen micellar solution.

Micro emulsions are widely used and are generally favored for their optical properties or ability to be absorbed by the skin or to cross membranes including biological membranes. Micro emulsions also form useful drug delivery systems, for example, including those which provide some level of protection of the active agent or provide prolonged release capabilities. In particular applications, the micro emulsion includes a solution in which solute molecules may be dispersed.

Various techniques are available for the production of micro emulsions. In general, micro emulsions are produced by emulsifying components under conditions including typically sufficient force or the required temperature to generate the required dispersion level, conductivity, viscosity, percolativity or other dispersion characteristics.

Micro emulsion formation can be assessed using scattering and spectroscopic techniques such as neutron scattering, time-average scattering, quasi-electric light scattering i.e., high-resolution ultrasonic spectroscopy or photon correlation spectroscopy. The partition coefficients of micro emulsions may also be measured chromatographically. The selection of particular formulations is based on a number of different paradigms depending upon the desired application. Illustrative paradigms include the hydrophilic-lipophilic balance, the phase-inversion temperature, or the cohesive-energy ratio. Micro emulsions may be formulated using a wide range immiscible liquids and other additional agents. Thermodynamically stable micro emulsions systems include those which are biocompatible. For example, oils suitable for use in forming a micro emulsion include vegetable oils, synthetic or natural triglycerides, fatty acid esters such as isopropyl myristate or ethyl oleate, and phospholipids such as lecithin or lysolecithin. Other organic liquids including, but not limited to, benzene, tetrahydrofuran, and n-methylpyrrolidone, or halogenated hydrocarbons, such as methylene chloride, or chloroform may also be used as the oil component of the micro emulsion. The proportion of oil or mixture of oils used in a micro emulsion is typically in the range about 10 and 60% by volume. Surfactants and co-surfactants are employed to enhance emulsion formation by altering the interfacial tension between phases and to enhance emulsion stability. Examples include anionic surfactants such as fatty acid soaps, acyl sulfates, or acyl sulfosuccinates; cationic surfactants, such as alkyl primary, secondary, tertiary, or quaternary amines; nonionic surfactants, for example, sorbitan esters or polyethoxylated esters of acyl acids, copolymers of polyethylene oxide and polypropylene oxide. The content of the surfactant or surfactants in a micro emulsion, can range, for example, from between about 0.1 to 60% by volume. Co-surfactants include aliphatic alcohols. Shorter chain alcohols, such as ethanol, are particularly preferred to more toxic, longer chain alcohols for use as co-surfactants. Alcohol content may range, for example, from about 0 to about 30% by volume in the micro emulsion. Solvents or other agents may also be employed to enhance emulsion formation or stability. Other agents may be introduced to provide functions such as pH, ionic content, polymerisation, smell, sterility, colour, viscosity etc. Micro emulsions may also be generated using any suitable synthetic plastic or polymeric, monomeric or hybrid colloidal material.

By whatever route or combination of routes, the active agent is administered in a therapeutically effective amount.

Pharmaceutical compositions are conveniently prepared according to conventional pharmaceutical compounding techniques. See, for example, Remington's Pharmaceutical Sciences, 18th Ed. (1990, Mack Publishing, Company, Easton, Pa., U.S.A.). The composition may contain the active agent or pharmaceutically acceptable salts of the active agent. These compositions may comprise, in addition to one of the active substances, a pharmaceutically acceptable excipient, carrier, buffer, stabilizer or other materials well known in the art. Such materials should be non-toxic and should not interfere with the efficacy of the active ingredient. The carrier may take a wide variety of forms depending on the form of preparation desired for administration, e.g. intravenous, oral or parenteral.

For oral administration, the compounds can be formulated into solid or liquid preparations such as capsules, pills, tablets, lozenges, powders, suspensions or emulsions. In preparing the compositions in oral dosage form, any of the usual pharmaceutical media may be employed, such as, for example, water, glycols, oils, alcohols, flavoring agents, preservatives, coloring agents, suspending agents, and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form, in which case solid pharmaceutical carriers are obviously employed. If desired, tablets may be sugar-coated or enteric-coated by standard techniques. The active agent can be encapsulated to make it stable to passage through the gastrointestinal tract. See for example, International Patent Publication No. WO 96/11698.

For parenteral administration, the compound may dissolved in a pharmaceutical carrier and administered as either a solution or a suspension. Illustrative of suitable carriers are water, saline, dextrose solutions, fructose solutions, ethanol, or oils of animal, vegetative or synthetic origin. The carrier may also contain other ingredients, for example, preservatives, suspending agents, solubilizing agents, buffers and the like.

The actual amount of active agent administered and the rate and time-course of administration will depend on the nature and severity of the burn injury. Prescription of treatment, e.g. decisions on dosage, timing, etc. is within the responsibility of general practitioners or specialists and typically takes into account the condition of the individual patient, the site of delivery, the method of administration and other factors known to practitioners. Examples of techniques and protocols can be found in Remington's Pharmaceutical Sciences, supra.

The pharmaceutical composition is contemplated to exhibit therapeutic activity when administered in an amount which depends on the particular case. The variation depends, for example, on the human or animal and the agent chosen. A broad range of doses may be applicable. Considering a patient, for example, from about 0.1 ng, 0.2 ng, 0.3 ng, 0.4 ng, 0.5 ng, 0.6 ng, 0.7 ng, 0.8 ng. 0.9 ng, or 0.1 mg, 0.2 mg, 0.3 mg, 0.4 mg, 0.5 mg, 0.6 mg, 0.7 mg, 0.8 mg, 0.9 mg to about 1 to 10 mg or from 5 to 50 mg of fetuin polypeptide or agent may be administered per kilogram of body weight per day. Dosage regimes may be adjusted to provide the optimum therapeutic response. For example, several divided doses may be administered daily, weekly, monthly or other suitable time intervals or the dose may be proportionally reduced as indicated by the exigencies of the situation. In some embodiments, the fetuin-containing compositions will generally contain about 0.01% to 90%, about 0.1% to 50%, or about 1% to about 25%, by weight of fetuin polypeptide, the remainder being suitable pharmaceutical carriers or diluents etc. In specific embodiments, an effective amount of fetuin polypeptide may be from about 0.0005 to 500 mg per mL of tissue volume to be treated or from 0.005 to 50 mg per mL of tissue volume to be treated or from 0.05 to 5 mg per mL of tissue volume to be treated.

The agents may be administered in a convenient manner such as by the oral, intravenous (where water soluble), intraperitoneal, intramuscular, subcutaneous, intradermal or suppository routes or implanting (e.g. using slow release molecules). The agent or composition comprising the agent may be administered in the form of pharmaceutically acceptable nontoxic salts, such as acid addition salts or metal complexes, e.g. with zinc, iron or the like (which are considered as salts for purposes of this application). Illustrative of such acid addition salts are hydrochloride, hydrobromide, sulfate, phosphate, maleate, acetate, citrate, benzoate, succinate, malate, ascorbate, tartrate and the like. If the active ingredient is to be administered in tablet form, the tablet may contain a binder such as tragacanth, corn starch or gelatin; a disintegrating agent, such as alginic acid; and a lubricant, such as magnesium stearate.

The present invention also provides kits comprising fetuin polypeptide or an agent from which fetuin polypeptide is producible. In some embodiments, the kits further comprise a debridement agent. Kits are contemplated which retain fetuin polypeptides or fetuin producing agents in a form suitable for subsequent administration. Thus the polypeptides or agents may be stored separately or together with components of compositions which render the polypeptides or agents suitable for administration. In one example, a fetuin polypeptide is separately stored in a kit in freeze-dried form and reconstituted prior to use with an aqueous buffer stored with a separate compartment in the kit. Other components optionally include gels, creams, ointments, powders, films, aerosols, emulsions, sealants, dressings and the like suitable for application to a dermal injury. In other embodiments, the components are sterilized. In yet other embodiment, the components of the kit further comprise preservatives, anti-bacterial anti-fungal agents etc.

The present invention is further described by the following non-limiting Examples.

### Example 1

### A Novel Model of Fetal and Lamb Response to Deep Dermal Injury Indicates that the Fetus Heals a Deep Burn Injury in a Scarless Fashion

It was hypothesized that a mid gestational fetus would heal a deep dermal burn injury in a scarless fashion, and a one-month old lamb would heal a similar injury with scarring. To test the hypotheses, models were developed of deep dermal partial thickness injury in the 80-day gestation (term=150 days) Merino fetus and the 1-month old Merino lamb. Subsequently, a study of the different healing modalities of these wounds was undertaken. Fetal and post-natal wounds were compared in three ways: macroscopically at post-mortem, with a novel histopathological scoring system and with immunohistochemistry. The expression of TGFβ1 and α-SMA proteins was examined. These proteins are markers of the myofibroblast, a cell strongly implicated in scar tissue formation.

A standardized model of deep dermal burn, as judged by light microscopy, was created on both the merino fetus and 1 month old lamb, using heated water. The injury pattern was recreated in a total of twenty-one fetuses and lambs. These animals were euthanased sequentially, and samples of skin were removed and tissue repair was assessed using light microscopy and immunohistochemistry for Transforming Growth Factor β (TGFβ1) and alpha smooth muscle actin (αSMA).

For the fetal model, merino ewes were super-ovulated with 500 IU Folligon (Intervet International) and time mated. At 80 days fetal gestation, the ewes were anaesthetized using thiopentone (10-15 mg/kg) and intubated with a size 10 Portex endotracheal tube. Anesthesia was maintained with halothane in 100% oxygen and continuous pulse oximetry monitoring was used throughout the procedure.

The ewe was placed in the supine position, the anterior abdomen was prepared by shaving and the area was swabbed with Betadine™. Following the creation of a paramedian skin incision, avoiding the superficial central abdominal vein, the uterus was delivered onto the operating field. A hysterotomy wound was fashioned, and the head and upper trunk of the fetus was delivered to the sterile field to conserve maximal quantities of amniotic fluid.

Polypropylene tubes of 15 mls volume were filled with sterile water and were heated in a hot water bath. The temperature of the water was measured using a digital thermometer (N19-Q1436 Dick Smith, Australia, range −50° C. to 100° C. (±0.5%)). Once the tubes had reached the chosen temperature they were removed in a sterile manner from the water bath and quickly inverted onto the flank of the fetus, care being taken to avoid spillage. To identify the margin of the burn area, the scalded areas (1.5 cm diameter) were marked by tattooing with sterilized ink (Rotring™, Germany) in a 27 G needle. The fetus was then returned to the uterus, which was closed, and after the laparotomy wound was sutured, the ewe was woken and returned to the sheep pen.

For the lamb deep dermal burn injury model, merino lambs (28-30 days old) were anesthetized with thiopentone (15 mg/kg) and intubated with a size 6.0 Portex endotracheal tube. Anesthesia was continued with halothane/100% oxygen mixture, and continuous pulse oximetry monitoring was used throughout the procedure. The lamb was shaved on a standard area of the lower abdomen, initially with sheep clippers then with a razor, lukewarm water and shaving cream, until the skin was free of wool. The wound was then gently washed with lukewarm water to remove any soap residue. Hot water for scalding was prepared as previously described, and applied in the same manner. Following the creation of scalds and tattooing of the wound margin with ink, the wounds were covered with paraffin gauze and soft cotton padding dressing (Melonin™-Smith and Nephew, Australia), to reduce direct trauma and soiling of the burned area. The lamb was then awakened and returned to a pen.

To establish the optimal temperature and duration of thermal injury, a total of 3 fetuses and 6 lambs were used with multiple scalds at varying temperatures on all animals. Scalds were created using the aforementioned method. In the fetal model, an initial temperature of 60° C. for 5 seconds, increasing in 2 second increments was the starting point, as this temperature is relevant in thermal injury in children (Moritz A, et al., (1947) Am. J. Pathol., 23:695-720). The temperature was also increased in 2° C. increments, up to a maximum of 72° C. At two days post operation, the ewes were euthanased using pentobarbital sodium 200 mg/ml (20%), and the duplicate samples of the 7 different temperature scalds were cut out within the wound border and fixed in 10% buffered formalin (10%). Tissues were then blocked in paraffin and 4 μm sections were taken and stained with hematoxylin and eosin. These sections were analyzed for depth of injury by a specialist fetal histopathologist, who was blinded to the temperatures used to create the injuries. A deep dermal partial thickness burn injury (DDPTBI) was defined as an injury causing cell death into the reticular dermis, but not causing total necrosis of the hair follicle bulb (Shakespeare P. G., (2001) Burns, 27(8):791-792). As the depth of the hair follicle bulb varies in sheep, multiple fields of view from the duplicate samples were assessed. A temperature of 66° C. for 7 seconds was found to give a consistent depth of injury in the fetus. Using the same process, a temperature of 82° C. for 10 seconds consistently created a deep dermal injury in the lamb model.

Following the successful development of these models, a total of fifteen fetuses and fifteen lambs were scalded in the above manner, using water at 66° C. for 7 seconds and 82° C. for 10 seconds for the fetus and lamb respectively. The animals were euthanased in a serial manner: three randomly selected animals each on days 1, 7, 14, 21 and 60. Discs of injured skin and control unburned skin (contralateral flank) were obtained from each animal to allow for direct comparison, initially using light microscopy. Tissue was also retrieved and stored for later analysis by immunohistochemistry, proteomic analysis and RNA analysis. Upon euthanasia, wounds were examined and photographed, and tissue was collected. Macroscopically, there were no burn wound infections in the lamb group, nor any sign of significant trauma.

Tissue sections were fixed in 10% buffered formalin and blocked in paraffin. Sections of 4 μm thickness were stained with haematoxylin and eosin. These sections were then examined in a blinded manner by the same histopathologist who assisted in the creation of the model. Between three and four sections on each slide were examined. To determine if the tissue was burned or control, a scoring system was developed to enable a more objective assessment. A total of five markers of injury were observed: number of fibroblasts, alteration of interstitial tissue, epidermal thickness, number of hair follicles and alteration in papillary and reticular dermis. These markers were graded from 0 to 3, with (0=normal and 3=severe abnormality). For each tissue section these scores were then added together to get a total score for each sample.

To validate this novel scoring system, the same histopathologist scored the slides on two separate occasions, between one and three months apart in a blinded manner. Once all slides were assessed and scored on the second occasion, the scores in each category were compared against those from the first assessment and a kappa statistic was determined which gives a measure of concordance between these scores. A kappa value greater than 0.4 is considered to show good concordance (Cohen J. (1960) Educ. Psychol. Meas., 20:37-46).

Paraffin sections (4 μm) of burn and control skin were immunohistochemically analyzed for α-smooth muscle actin (α-SMA) and transforming growth factor beta 1 (TGFβ1). Slides were de-waxed, hydrated and washed with Tris-buffered Saline (TBS). The TGFβ1 slides then underwent an antigen retrieval step of 0.1% trypsin in Phosphate Buffered Saline (PBS) at 37° C. for 15 min. All slides had an endogenous peroxide removal step (0.6% H₂O₂ in TBS, 10 min) followed by blocking in 4% powdered milk in TBS (α-SMA) and/or 1:10 goat normal serum in 1% BSA (bovine serum albumin) in TBS (α-SMA and TGFβ1) for 20 min. The primary antibodies used were: Sigma monoclonal mouse anti-α-SMA (#A2547) 1/400 in 1% BSA in TBS for 90 minutes, and Santa Cruz rabbit anti-TGFβ1 (#sc-146) 1/40 in 1% BSA in TBS overnight at 4° C. The secondary antibodies used were: DAKO goat anti-mouse-HRP (#PO447) 1/100 for 60 min (for α-SMA) and DAKO “Envision+ System/HRP, Rabbit” (#K4003) neat for 60 min (for TGFβ1). Development was with Zymed DAB (3′-Diamino benzidine) (#2014) for 2 min (α-SMA) or 5 min (TGFβ1) followed by a counterstain with Hematoxylin. A negative control was used for each protein that consisted of no primary antibody.

To enable an objective and quantitative scoring system, slides were then visualized using a Nikon EP600 microscope fitted with a Spot RT slider cooled CCD camera and captured directly as digital images. Up to five fields were captured from each slide, and all slides were photographed on the same day to avoid any variability associated with the light source. Image morphometry was analyzed using ImagePro Plus® image analysis software (Version 4.1.29, Media Cybernetics, L.P, USA) which can automatically calculate the area (microns²) of stained protein (brown DAB) in each of the sections. The TGFβ1 was mainly prevalent in the epidermis, so only the protein within the epidermal border was counted. Alpha-SMA is present in blood vessels and the erector pili muscle associated with hair follicles, as well as in myofibroblasts. To ensure only interstitial α-SMA was included, counting was performed inside 50 μm square boxes positioned within the interstitial area (avoiding epidermis, hair follicles and blood vessels). The average of twelve of these boxes was used for each field, with up-to five fields viewed per slide.

Data for fetuses and lambs were analyzed separately by analysis of variance using hierarchical models to partition variability according to different levels of replication and sampling. At the highest level, differences were tested against variability between replicate animals. At the next level, differences between burn and control treatments and the interaction of this comparison with days were tested against the residual variation from the split plot design at the tissue level. These steps were all that were required for analysis of light microscopy. In the immunohistochemical assessment sampling variability was estimated from the differences between the five fields for each tissue sample. In the case of αSMA, variation between the twelve boxes for each field was used to estimate the sub-sampling variation. Variance components were estimated by equating expected mean squares with estimated mean squares at each level of the hierarchical model. These variance components were then used in sensitivity analyses to assess the impact of the changes in the number of animals, tissue samples, field and boxes on the variance of the population mean.

A reproducible deep dermal burn injury was created in the fetus by application of water at 66° C. for 7 seconds, and at 82° C. for 10 seconds to the lamb. Macroscopically, it was difficult to determine the area of scald in the fetus following day 7 post injury. It was always possible to identify the area of scald in the lamb. Using a 5-point histopathology scoring system for alteration in tissue morphology, it was impossible to determine a difference between control and scalded skin in the fetal model after day 7 but was possible in the lamb at all stages. There were large differences in content of αSMA and TGF-β1 between control and scalded lamb and these differences were statistically significant at day 14 post burn injury p<0.05 respectively. Analysis of variance of the model indicated that fewer images could be captured with minimal increase in variability in future use of this model.

At fetal post mortem, the wound was visible as a hyperemic circle at day 1 (and day 2 during the creation of the model) but by day 7 and subsequently thereafter the black ink tattoo was the only method of identifying the scalded area. On several fetuses at day 14 and 21, there was a well circumscribed area of soft exudate overlying the scald area. This fell away on gentle lavage with normal saline to reveal uninjured skin beneath. The scald area was covered by wool at day 60.

On post mortem of the lambs, the areas of scald were easily identified at all time points. Eschar was evident up to day 21 post injury, and absent by day 60. After removal of the eschar, palpable elevated contracture was evident in the center of the scalded area. Due to the regeneration of wool and relatively small size of injury, it was occasionally difficult to identify the scald at day 60. No attempt was made to shave the wool off, as this may have induced further tissue trauma.

The histopathologist's composite score for all samples were compared against each other, using Kappa measurement of agreement, which provides a co-efficient of agreement for nominal scales (Cohen J. (1960) supra). The Kappa value in the fetal samples was 0.53 (standard error 0.049), which was lower than that for the lambs, at 0.742 (standard error 0.042) (Cohen J. (1960) supra). The composite scores for the five markers of injury of fetus and lamb triplicate samples were compared at each time-point for burn and control skin.

In all samples of lamb tissue, there was a statistically significant difference in scores between burnt and control samples at all time periods, indicating that the histopathologist could determine differences between control and scalded lamb tissue. In the fetus, there was a statistically significant difference between burn and control scores at Day 1, but no statistically significant difference following that. Thus, the injury created on the lamb was histopathologically detectable throughout the study period, but was histopathologically undetectable after Day 1 in the fetus.

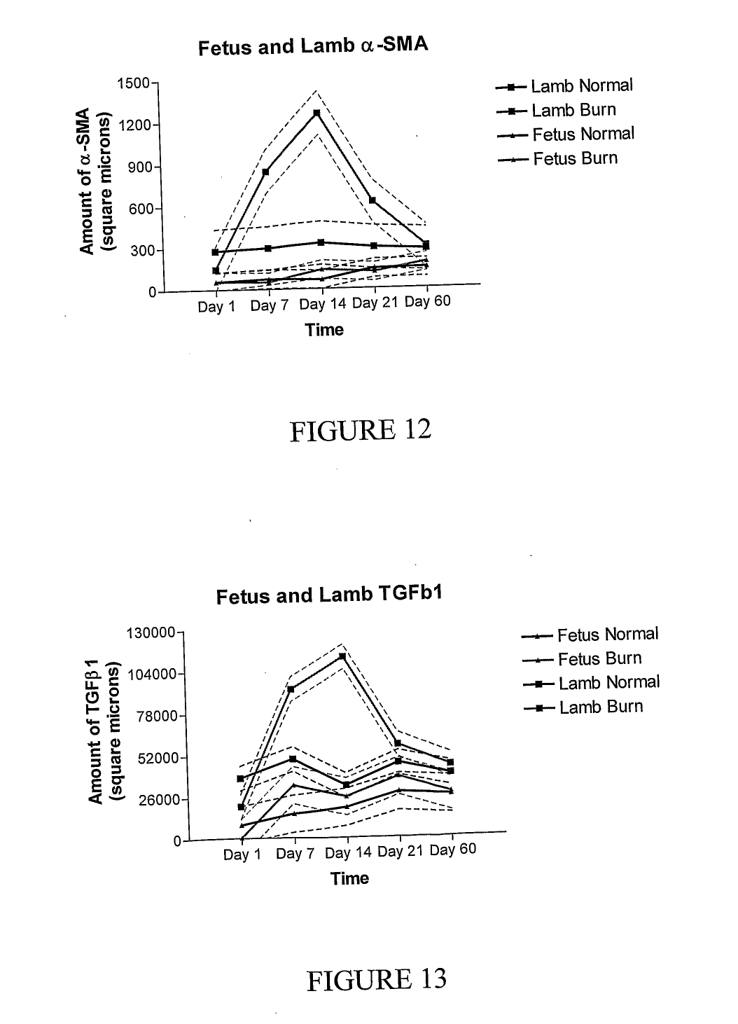

The amount of αSMA was calculated and the results were expressed as average±standard error mean. As shown in FIG. 12, thermal injury resulted in no significant differences in quantity of αSMA in the fetal model, at any stage post injury, nor were there any significant differences in content of αSMA between control samples of fetal and lamb skin. However, there were significant differences in αSMA content of the burnt and control lamb at all stages, other than day 60.

The amount of TGFβ1 in the epidermis was calculated per unit area and the results for all triplicates were expressed as average±standard error mean. As shown in FIG. 13, the epidermis of all lamb samples at day 7 and 14 was hypertrophied, and this was reflected in the total content of TGFβ1 in the lamb burned samples. Again, no significant differences of content of TGFβ1 were observed between fetal burnt and control skin, but statistically significant differences in the content of TGFβ1 in the lamb burnt and control samples were found until day 60 post burn injury.

There are a number of levels of variability inherent within this study. In assessment of αSMA, the four levels of variability are: the number of animals, number of treatments (control and experimental), number of micrographs assessed and number of areas assessed from each micrograph. This variability may be quantified using the equation:

V=(σ1²/r1)+(σ2²/r1r2)+(σ3²/r1r2r3)+(σ4²/r1r2r3r4)

In the assessment of αSMA, multiple sections were chosen (to avoid including αSMA within blood vessels etc) In assessment of TGFβ1, one area (total epidermis) was selected in each slide, therefore the equation to represent variability was simplified to:

V=(σ1²/r1)+(σ2²/r1r2)+(σ3²/r1r2r3)

Where

R1=number of animals (15)

R2=number of treatments

R3=number of pictures (5)

R4=number of samples (12)

Values as calculated for σ are shown in Table 2. There are no σ4 values for TGFβ1 as total epidermis was assessed.

These equations assisted in calculating the relative importance of the four variables to the statistical results, and a means to determine the magnitude in variability that would result if each variable were altered.

The αSMA results provide a base figure of 25.4 from this calculation. If the number of animals is doubled, this variability drops to 12.7, or 50% of original. If the number of animals used is halved, the variability doubles to 54.6, or 200%. If 10 times the number of animals are used, variability drops to 2.54, or 10% of the original. However, other components of the equation, such as number of pictures and number of samples make a substantially less significant change in variability if their numbers are altered. If the number of images captured is halved, variability changes to 28.3 (or 111% of baseline) or doubled becomes 24.5 (or 96.5%). Again, if the number of samples from each image is halved, variability changes to 26.04/102%; if 10 times the number of samples from each image are taken, variability changes to 24.99/98%. This calculation thus allows us to modify and optimize efficiency in further similar studies.

It has long been recognised that the fetus heals an incisional wound in a scarless fashion. However, the pathophysiology of the thermal injury and its repair is so far removed from an incisional wound that it would be imprudent to assume the two injuries heal in a similar fashion. The model described here provides the first proof that the fetus heals a deep dermal burn injury in a scarless fashion, and the lamb heals a similar wound with scarring. Although the temperatures and duration of injury required to create the injuries in the fetus and lamb are different (67° C./7 seconds and 82° C./10 seconds respectively), the histological injuries and depth of burn are similar. This relationship is equivalent to that seen in human children and adults, first described by Moritz and Henriques, and is the result of the difference in skin thickness and tensile strength (Moritz A, et al., (1947) supra). The temperatures required to inflict a deep dermal burn are much higher in the present study than those reported in that previous work, which was unsupported by histopathological assessment.

This model provides a vehicle to compare the healing mechanisms of the ovine fetus and lamb in response to deep dermal burn injury. The macroscopic, histological and immunohistochemical analysis all support the hypothesis that the fetus heals a deep dermal partial thickness burn injury in a scarless fashion, while the lamb heals with scarring. The validated histological scoring system, which examined five parameters recognized to be integral in wound repair, confirmed the initial macroscopic observation that evidence of scar formation was not present in the fetus from day 7 post burn, but was present in the lamb throughout all time points examined. The statistically significant differences seen in assessment of burnt fetus versus control at day 1 were as a result of the recent thermal damage and tissue necrosis, and not of tissue repair.

### Example 2

### Ovine Fetal Model for Protein and Nucleic Acid Analysis

Merino ewes were super-ovulated with 500 IU Folligon (Intervet International) and time mated. At 80 days fetal gestation, the ewes were anesthetized using thiopentone (10-115 mg/kg) and intubated with a size 10 Portex endotracheal tube. Anesthesia was maintained with halothane in 100% oxygen and continuous pulse oximetry monitoring was used throughout the procedure.

The ewe was placed in the supine position, the anterior abdomen was prepared by shaving and the area was swabbed with Betadine™. Following the creation of a paramedian skin incision, avoiding the superficial central abdominal vein, the uterus was delivered onto the operating field. A hysterotomy wound was fashioned, and the head and upper trunk of the fetus was delivered to the sterile field to conserve maximal quantities of amniotic fluid. Polypropylene tubes of 15 mls volume, with a 1.5 cm diameter were filled with sterile water and were heated in a hot water bath. The temperature of the water was measured using a digital thermometer (N19-Q1436 Dick Smith, Australia, range −50° C. to 100° C. (±0.5%)). Once the tubes had reached the chosen temperature they were removed in a sterile manner from the water bath and quickly inverted onto the flank of the fetus, care being taken to avoid spillage. To identify the margin of the burn area, the scalded areas were marked by tattooing with sterilised ink (Rotring, Germany) in a 27 G needle. Three burns were created on the anterior flank of the fetus then it was returned to the uterus, which was closed. The majority of the ewes were carrying twins and the twin animal was subjected to a sham operation and acted as a control.

After the laparotomy wound was sutured, the ewe was woken and returned to the sheep pen. When initially creating the model it was determined that to create a deep dermal partial thickness burn injury, water should be used at 66° C. for 7 seconds in the fetus. This was determined by a series of histopathological analyses by an experienced fetal pathologist. Fetuses were euthanized at days 1, 7, 14, 21 and 60 days post burn. Skin disks were harvested at these time points and two were frozen for protein and RNA while the third was stored for frozen and paraffin blocks.

### Example 3

### Lamb Model for Protein and Nucleic Acid Analysis

Merino lambs, (28-30 days old) were anesthetized with thiopentone (15 mg/kg) and intubated with a size 6.0 Portex endotracheal tube. Anesthesia was continued with halothane/100% oxygen mixture, and continuous pulse oximetry monitoring was used throughout the procedure. The lamb was shaved on a standard area of the lower abdomen, initially with sheep clippers then with a razor, lukewarm water and shaving cream, until the skin was free of wool. The wound was then gently washed with lukewarm water to remove any soap residue. Hot water for scalding was prepared as previously described, and applied in the same manner.

For the lamb skin. it was determined that water should be applied at 82° C. for 10 seconds to create a deep dermal partial thickness burn. This was also determined by histopathological analysis. A higher temperature and longer duration of heat is required to create the same injury in lamb skin compared to fetal skin because the lamb skin is thicker and has the presence of wool follicles. Following the creation of scalds, and tattooing of the wound margin with ink, the wounds were covered with paraffin gauze and Melonin™ soft cotton dressing (Smith and Nephew, Australia), to reduce direct trauma and soiling to of the burned area. The lamb was then awakened and returned to a pen. The animals were euthanized in a serial manner: 3 randomly selected animals each on days 1, 7, 14, 21 and 60. Discs of injured skin and control unburnt skin (contralateral flank) were obtained from each animal to allow for direct comparison, initially using light microscopy. Tissue was also retrieved and stored for later analysis by immunohistochemistry, proteomic analysis, RNA analysis and electron microscopy.

### Example 4

### 2D Gel Electrophoresis

Approximately 300 μg total protein was obtained from half of a 1.5 cm diameter disc of fetal or lamb skin. Tissue was ground in liquid nitrogen and proteins dissolved in RIPA buffer (50 mM Tris HCL, 150 mM NaCl, 1 mM EDTA, 1% Triton X100, 1% Sodium deoxycholate, 0.1% SDS) containing Complete™ (Roche) protease inhibitor cocktail. Samples were subjected to 3 freeze/thaw cycles in liquid N₂, sonicated (3×10 sec) then centrifuged at 12000×g for 10 minutes. The supernatant was purified with a Plus One 2D gel Clean-up Kit (Amersham Bioscience) according to manufacturer's instructions. The samples were then dissolved in sample buffer (9M Urea, 65 mM DTT, 2% Pharmalyte3-10 IPG, 0.5% Triton X100), and protein concentration was determined by the BCA method (Pierce) using a re-precipitated sample aliquot.

For isoelectric focusing 20 μg protein in 10 μl sample buffer from each of the samples were mixed with 115 μl rehydration solution [8M Urea, 0.5% Triton X-100, 0.5% Pharmalyte 3-10 IGP, 13 mM DTT, bromophenol blue] and applied onto a 7 cm 3-10 Non Linear immobilized pH gradient (IPG) strip (Amersham Bioscience) in the Immobiline™ DryStrip Reswelling Tray (Amersham Bioscience). Strips were overlaid with mineral oil (Amersham Bioscience) for reswelling overnight.

IEF was performed on the Muliphor II flatbed unit (Pharmacia Biotech). The rehydrated IPG strips were placed onto plastic aligner in an Imobiline Strip tray (Pharmacia Biotech). Moistened electrode filters were placed across the cathodic and anodic ends of the IGP strips and the electrodes aligned and overlaid with mineral oil. The unit was connected to an EPS 3500 power supply (Pharmacia Biotech) and run at 3 different cycle settings: 1) 5 minutes, 200V, <1 mA 2) 1.5 hrs, 3500V, <1 mA and 3) 1 hr, 3500V, <1 mA. Instrument temperature was kept at 20° C. by circulating tap water.

Prior to the 2^{nd} dimension the IPG strips were reduced for 15 minutes in 10 ml equilibration solution (50 mM Tris-HCL, 6M Urea, 30% v/v Glycerol, 1% SDS) containing 30 mM DTT and alkylated for a further 15 minutes in 10 ml equilibration solution of 240 mM iodoacetamide containing grains of bromophenol blue. The second dimension SDS-PAGE gel was performed on a NuPAGE 4-12% Bis-Tris Zoom® gel (Invitrogen). A protein bench marker (Invitrogen) was applied to the marker well. IPG strips were sealed with agarose at the top of the SDS-PAGE gels. Gels were run in an XCell SureLock™ Mini Cell system (Invitrogen) in 1×MOPS running buffer for 50 minutes at 200V. Gels were then fixed for 20 minutes with 20% TCA and subsequently silver stained using the standard Heukeshoven and Dernick method. The gels were developed for 8 minutes.

2D gels were conducted on all replicates and controls for both the fetal and lamb skin disk protein extracts. The gels showed a remarkably consistent pattern of protein spots between samples taken from replicate animals from the same treatment groups. A representative grouping of control fetal gels is shown in FIG. 1 where gels from three separate control animals are displayed.

Differentially expressed proteins were identified by scanning the silver stained gels, assigning them different colours and overlaying them in Adobe Photoshop. A transparency of the gel showing spots of interest was printed and used to identify these spots for extraction. The most obviously differentially expressed protein is indicated by arrows in FIG. 2. This protein is present at much higher concentration in fetal control protein extracts than in the lamb control. It also has a much higher concentration in the 14 day post burn fetal sample than in the 14 day post burn lamb sample.

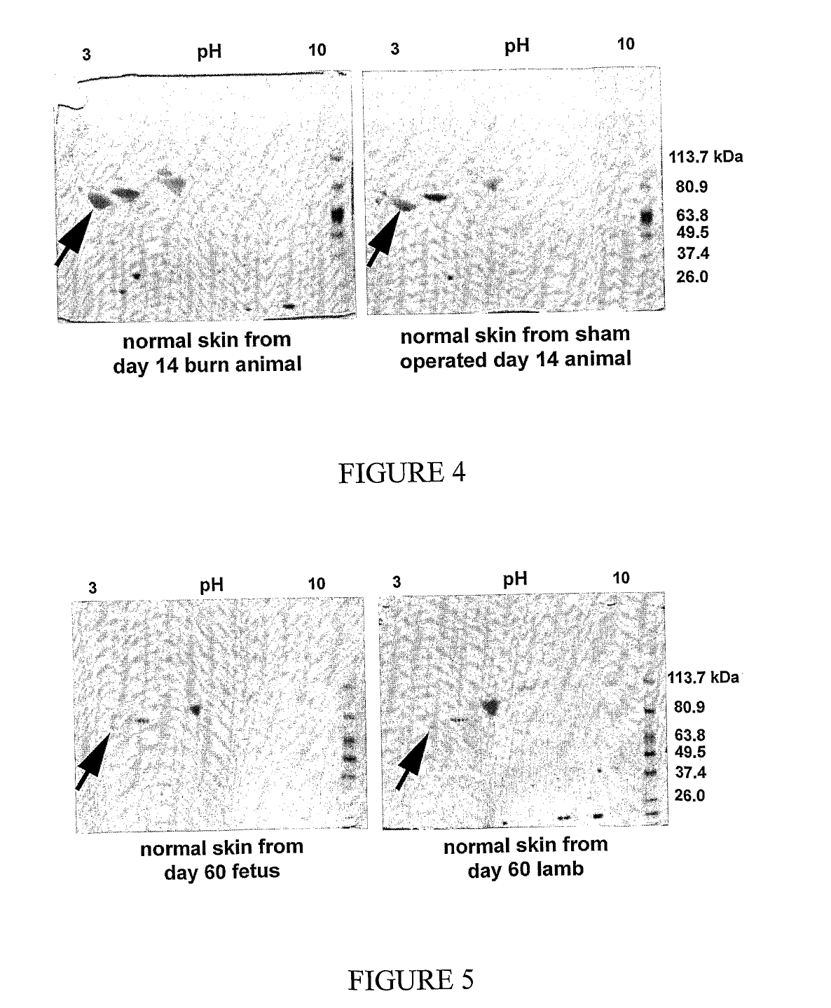

To identify whether the protein concentration rose in response to the burn injury, 2D gels were run on skin extracts from normal unburned skin from a burned animal and a fetus of the same gestation as the 14 day post burn animal which had not been burned or operated on (day 94 gestation). These gels are shown in FIG. 4. These results suggest that the level of fetuin in the skin changes in a gestational manner rather than as a response to burn or the operation.

To further demonstrate gestational age changes, gels were run from samples 60 days after burn, but from normal skin. This tissue is from 140 days gestation (term is day 145-150).

FIG. 5 shows that these gels have decreased levels of fetuin, compared to earlier timepoints. The fetal scar-free timepoint during gestation has been reported to be around day 100-120. After this time, fetuses are known to heal without scar, whereas before this point they heal with scar. Here it can be seen that fetuin is greatly upregulated during the “scarless” time and is down-regulated during the “scarring” time.

In humans fetuin A (α₂-HS glycoprotein) is maximal at the 10^{th} week of gestation and falls progressively after the 22^{nd} week to 1:40^{th} concentration at term (Yachnin S., 1975, J. Exp. Med., 141(1):242-256). This point in gestation coincides with the scar free timepoint described by others, after which the fetus loses its scar free healing ability. (Bullard K. M. et al., 2003, World J Surg 27(1):54-61; Colwell A. S. et al., 2003, Front Biosci., 8: s1240-1248; Ferguson M. W. et al., 2004 (supra)).

### Example 5

### In-Gel Digest of Silver Stained Gels

Spots of interest were excised with a blue tip of an Eppendorf pipette and transferred into an Eppendorf tube. Gel pieces were destained with a 1:1 mix of 30 mM potassium ferricyanide and 100 mM sodium thiosulfate and washed twice with ddH₂O. Dehydration was performed twice for 10 minutes in 50% acetonitrile containing 25 mM ammonium bicarbonate and dried in a Speedivac (Labconco) vacuum centrifuge for 30 minutes. Reduction was performed by incubation with 25 mM Ammonium bicarbonate containing 10 mM DTT for 1 hr at 56 C. Alkylation was performed with 25 mM Ammonium bicarbonate containing 55 mM iodoacetamide for 45 minutes at room temperature. The samples were washed again with Ammonium bicarbonate and dehydrated in 50% Acetonitrile (ACN)/25 mM Ammonium bicarbonate before being dried in a speed vacuum for 30 minutes.

Trypsin digestion using proteomic grade trypsin (Sigma) was for 1 hr at 4° C. in 20 ng/μl trypsin in 25 mM Ammonium bicarbonate. Trypsin excess was removed and 25 mM Ammonium bicarbonate was added for enzyme cleavage overnight at 30° C. To extract the peptides, the samples were shaken vigorously in 0.1% trifluoro acetic acid (TFA) (Sigma), followed by a pulse spin and the excess collected. After a repeated extract with 50% ACN/5% TFA, the collected extracts were speed vacuum dried and resuspended in 5 μl of 80% ACN/0.1% TFA.

### Example 6

### Mass Spectrometry

Protein spots were excised from the replicate gels for both fetus and lamb, trypsin digested and subjected to MALDI TOF and MALDI MS/MS analysis and was identified with high reliability as ovine Fetuin A.

Digested peptide mixture (1 μl) was spotted onto the MALDI target plate with 1 μl matrix (10 mg/ml αcyano-4-hydroxyl-cinnamic acid, Sigma). A Voyager MALDI-TOF mass spectrometer (Applied Biosystems) was employed for peptide mass mapping. For further identification the Q-Star MALDI MS-MS (Applied Biosystems) was used and identification was achieved by sending the results to Swiss Prot database using the Mascot Search Program.

### Example 7

### Immunohistochemistry

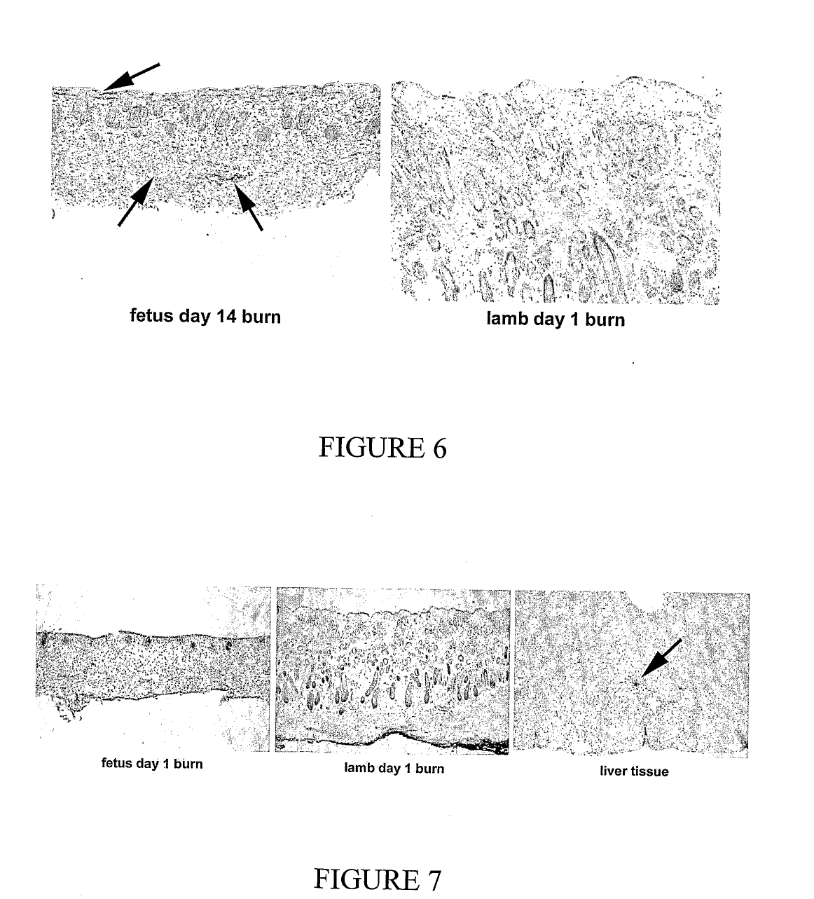

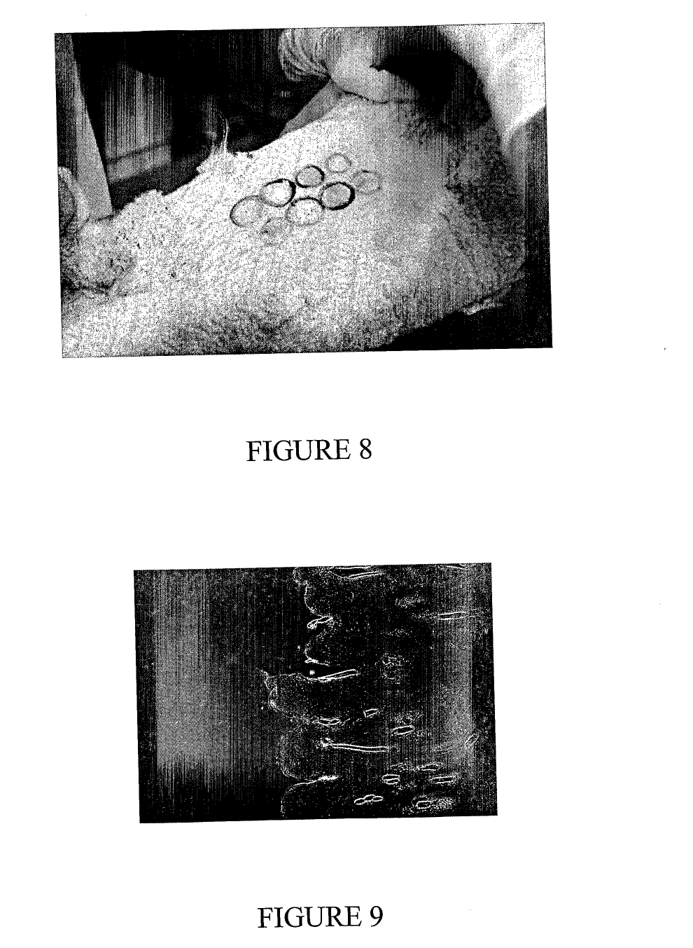

The distribution of fetuin in control and post burn fetal and lamb tissue was examined using immunohistochemistry. FIG. 6 shows staining for fetuin (DAB) in these tissue sections. Fetuin appears to be predominantly expressed in the fetal blood vessels, which may indicate that fetuin acts systemically. However, it is also found in the fetal tissue, suggesting that it may migrate from the vessels into the tissue. Fetuin is found in both burned and control tissue but stained less strongly in the lamb tissue compared to the fetal tissue.

Immunohistological staining was performed with fetal and lamb skin sections at different time points from the model described in Example 1. Sections were dewaxed in xylene and rehydrated through a descending alcohol series, followed by washing in TBS. Sections were quenched in H₂O₂ for 10 min, washed, and then blocked using 10% swine serum. The primary antibody (anti-bovine, rabbit polyclonal) was a kind gift from K.M. Dziegielewska (Melbourne) and was used at 1:500 in TBS 1% BSA overnight at 4° C. The Envision anti-rabbit antibody system (Dako) was used followed by development with DAB (Zymed) for 5 minutes. A 1 minute counterstain with hematoxylin was performed and the sections were mounted in Depex after a series of ascending alcohol series and xylol was performed. A Nikon EP600 microscope was used to view the slides. Sections were captured with a Spot RT slider cooled CCD camera as digital images at ×100 magnification.

### Example 8

### In Situ Hybridization

In situ hybridization was performed to ascertain whether fetuin was expressed in the skin in control skin or in response to burn. FIG. 7 shows the results of In situ hybridization on fetal and lamb skin 1 day post burn, compared to a positive control (liver tissue). There was some staining seen in hair follicles of the lamb skin, but little elsewhere. These results show that fetuin is either expressed very slightly or not at all in fetal or lamb skin and must be expressed elsewhere in the body.

A probe to detect sheep fetuin was modified from the sequence in (Dziegielwska et al. J Neurocytology 22:266-72) so that it was specific for sheep.

5′ GTGGGGTCC AGTACGTGGC AGGTGGTTTC CAGGGTATCT
AT 3′

A 5′ biotinylated probe of 41 bases was ordered from Sigma Genosys (Australia). Paraffin sections of lamb skin from the model developed previously (Fraser J. F. et al., 2005 (supra)) were stained for the presence of fetuin. Sections of sheep liver were used as positive controls. Paraffin sections were dewaxed, rehydrated and post-fixed in 4% paraformaldehyde for 10 min. Endogenous peroxidases were quenched with a 30 min wash in 0.3% H₂O₂ and slides were incubated in 0.1 M triethanolamine (TEA), pH 8.0 containing 0.25% acetic anhydride for 10 min. Cells were permeabilized by incubating in 2 μg/mL TE buffer RNase-free Proteinase K (Sigma). Slides were then pre-hybridized in a humidified chamber for 5 hours at 37° C. in buffer: 2×SSC, 10% dextran sulfate, 10×Denhardt's solution, 20% deionised formamide, 250 μg/mL denatured and sheared herring sperm DNA and 0.5% Tween 20. Hybridization was overnight at 37° C. in the same buffer, containing 50 ng/mL fetuin probe. The next day, slides were washed in 1×SSC, 20% formamide, 0.5% Tween 20 for 10 min at 37° C. followed by washing in 1×SSC for 15 min at 37° C. Sections were then incubated in streptavidin-HRP (1:300 dilution) (Zymed) for 30 min, followed by development using DAB (Zymed). The sections were then counterstained with hematoxylin and viewed under a Nikon EP600 microscope. Images were captured with a Spot RT Slider cooled CCD camera.

### Example 9

### Fluorescent Labeling of Fetuin, Cream Preparation and Application to Burn to Determine the Best Carrier for Fetuin

Lyophilized bovine Fetuin (Sigma) was dissolved in purified water at a concentration of 10 mg/mL. Aliquots of 200 μL were labeled using a FluoReporter Oregon Green® 488 Protein Labeling Kit (Molecular Probes). Assuming 85% recovery from the kit, the solution was dried down using a vacuum concentrator (Labconco) and added at 2 mg/mL to one of 3 different carriers: paraffin cream, aqueous cream (Orion, Australia) or dissolved in purified water to make an aqueous solution.

Two Merino lambs were scalded under general anesthesia as described previously. Eight wounds were created on each animal; 2× control wounds with nothing added, 2× aqueous solution treated wounds, 2× aqueous cream treated wounds and 2× paraffin cream treated wounds. The wounds were dressed with Melolin and Sleek and the lambs awoken and returned to the pens. 24 hours later the lambs were euthanized and the burn wounds (as well as an unburned area) were collected and fixed in formalin for paraffin embedding. Sections of (4 μm) were taken and examined under a Nikon EP epifluorescent microscope to determine the extent of fetuin penetration in each of the carriers. Brightfield transmittance and Fluorescent green images were captured with a Spot RT slider cooled CCD camera and overlayed with using Photoshop software.

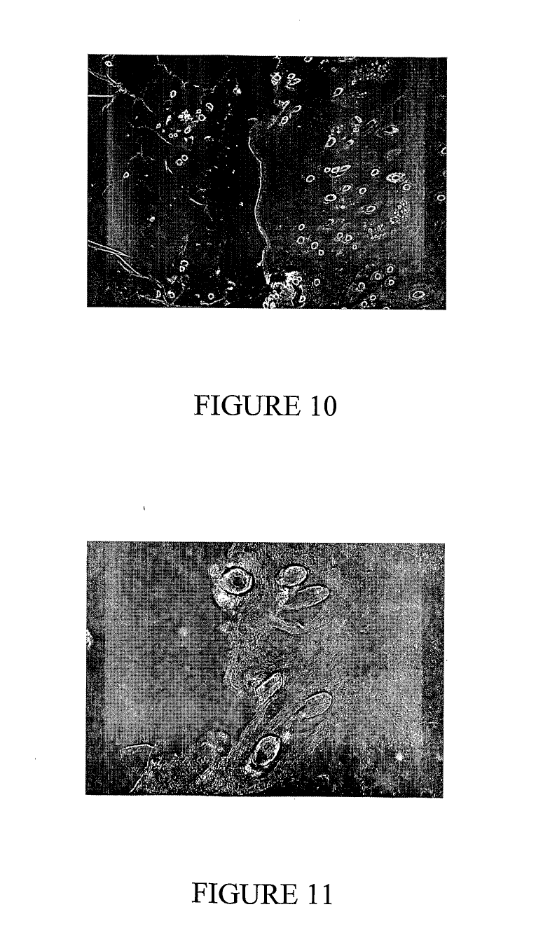

The fluorescently labeled fetuin could be seen clearly in the lamb tissue. There was little or no penetration of the fetuin in the oil based paraffin cream. However there was good penetration of the fetuin in the aqueous solution (FIG. 9) and the water based cream (FIGS. 10 and 11). As the aqueous cream was easier to apply to the skin surface, the aqueous cream was the carrier of choice.

### Example 10

### Testing of Fetuin on Burn Wounds

Testing of fetuin or a biologically active portion thereof, or a variant or derivative of these will be done using a newly developed porcine deep dermal partial thickness burn model. In this model 8.5 cm diameter burns are produced and scars which appear hypertrophic have been observed in animals 99 days post burn.

The porcine model has been developed because it provides a relatively large burn in skin recognized as the closest to human skin. While it is shown herein that topical application of fetuin in an aqueous cream enables penetration to the base of the hair follicles, other activities of the protein such as modification of macrophage response are likely to necessitate penetration deeper into the wound. Therefore the efficacy of topical applications are determined, as well as the best vehicle and concentration of application. It is also determined whether prolonged topical application allows penetration to the deeper wound bed and whether intradermal application of agent is efficient. Such experiments are conducted by the skilled address using routine procedure or as described herein without undue experimentation.

### Example 11

### Fetuin Enhances In Vitro Wound Healing More Efficiently than Thyroglobulin or Peptide TGF-β-1 Receptor Inhibitor

The “scrape” in vitro wound healing model has been previously presented in the literature (Li, F. et al., 2000, Exp Cell Res., 258(2): 245-253; Firth J. D. et al., 2004, J. Invest. Dermatol., 122(1): 222-231). In this model a scrape across a confluent monolayer of cells is used to simulate a wound and the effect of agents added to serum free media is tested on the rate of closure of the gap.

Human epidermal keratinocytes (HaCaT cells) were plated at a density of 1×10⁵ cells/well into Nunc 6 well plates in Dulbecco's Modified Eagle Medium (DMEM) containing 10% fetal bovine serum (FBS) benzyl penicillin (100 IU/ml), streptomycin (100 mg/ml) and fungizone (250 mg/ml). After confluence (2 days) monolayers were “wounded” with a 1 ml pipette tip (0.5 mm width) by scraping the tip across the centre of the well. The wells were then washed 5 times with DMEM. One ml of either DMEM with 10% FBS, DMEM or DMEM with: 3, 6, 12 or 24 mgs/ml bovine fetuin A (Sigma), 6.25, 12.5, 25 or 50 μg/ml peptide TGF peptide antagonist (Corresponding to residues 41-65 of TGFβ-1-3:—Huang S. S. et al., 1997, Journal of Biological Chemistry, 272(43):27155-27159) and 2.1, 4.2, 8.4 and 16.8 mg/ml bovine thyroglobulin was added to each well (2 replicates per plate, 4 plates). These concentrations were calculated to have equivalent antagonistic effect on the binding of TGFβ-1 to its receptor for each of the compounds. All DMEM solutions used contained benzyl penicillin (100 IU/ml), streptomycin (100 mg/ml) and fungizone (250 mg/ml). A mark was placed on the base of each well at right angles to the “wound” and the wound was photographed at the intersection of the mark and wound with a Nikon EOS 300 digital camera fitted to a JenaMed (Ziess Jena) inverted microscope (50×). Images were taken at the same positions 24 and 48 hours after addition of the treatment media. Digital images were analysed with Image Pro (Media Cybernetics) image analysis software and an image of a Nikon stage micrometer was used for calibration of length measurements. Results were summarized as the wound width after 24 and 48 hours.

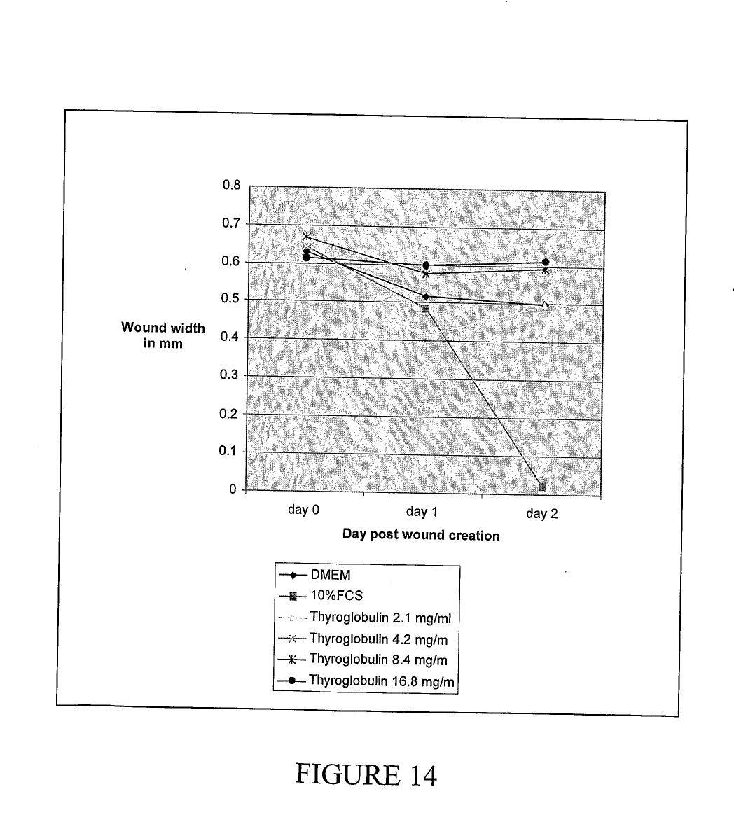

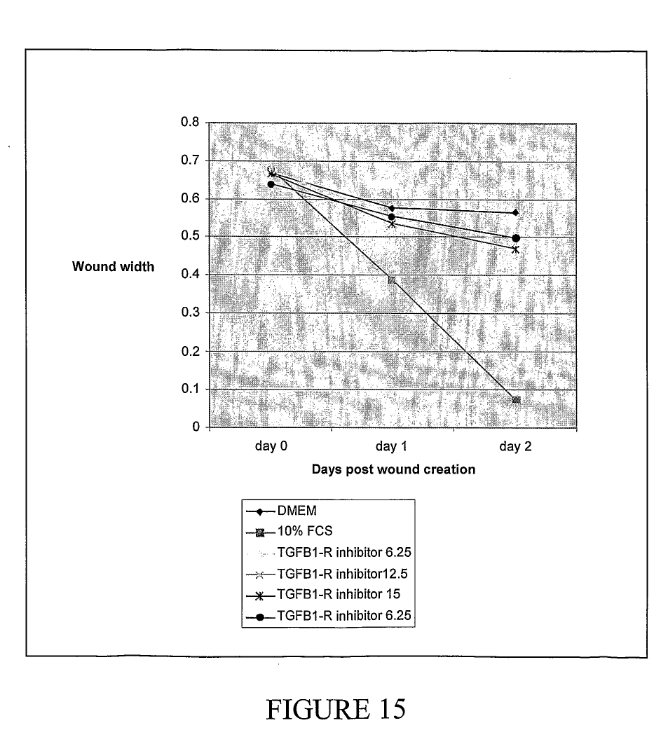

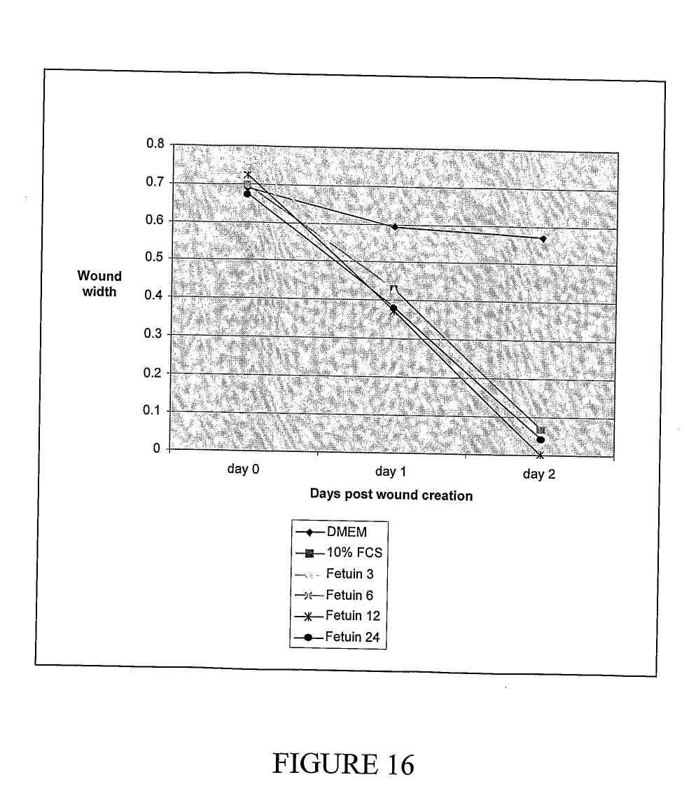

The results are tabulated in Table 3 and represented graphically in FIGS. 14, 15 and 16. FIGS. 14 and 15 show the effects of thyroglobulin and a peptide TGFβ1 receptor inhibitor on wound closure, respectively. FIG. 16 shows the effect of fetuin A on wound closure. At all concentrations of agents, fetuin A exhibited a significantly greater ability than thyroglobulin and TGFβ1 to enhance keratinocyte wound closure.

### Example 12

### Testing Skin Delivery Preparations and Skin Preparation

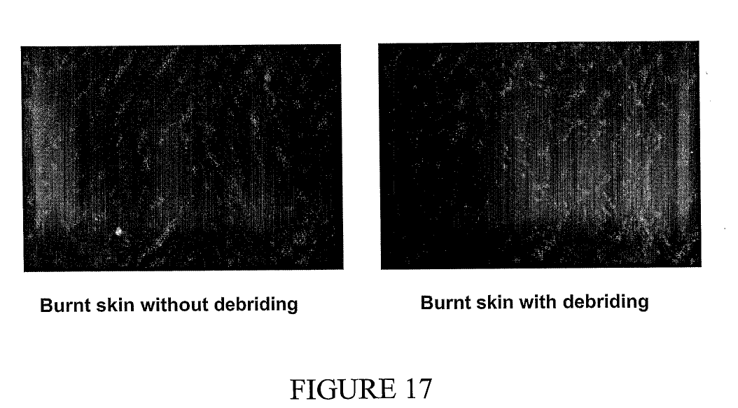

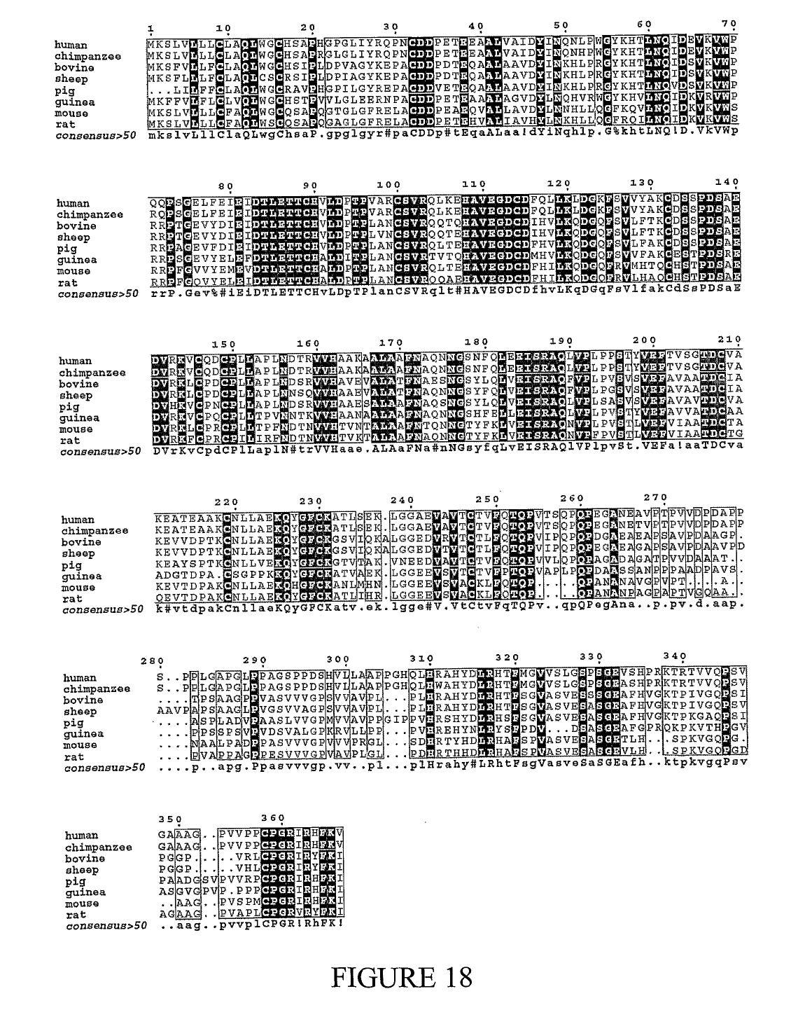

The potential use of the protein as a topical application was explored by labeling the protein with FluoroReporter Oregon Green® 488 Protein (Molecular Probes) then applying it as 1) a hydrophobic cream 2) a hydrophilic cream and an aqueous solution. These were applied to a porcine burn model and skin samples were collected 4 hours after application. Penetration of the protein into the wound was observed by epifluorescent microscopy. The aqueous applications were found to provide best penetration of debrided skin or when directly injected into the wound bed (FIG. 17).

### Fluorescent Labelling of Fetuin A and Treatment Preparation

Lyophilised bovine Fetuin A (Sigma) was dissolved in purified water at a concentration of 10 mg/mL. Aliquots of 200 μl were labelled using a FluoroReporter Oregon Green® 488 Protein Labelling Kit (Molecular Probes) after the manufacturer's instructions. The resulting solution was dried down using a Labconco Centrivap concentrator and weighed. Sufficient carriers (paraffin cream (Orion, Australia), aqueous cream (Orion, Australia) or purified water) were added to produce a final concentration of 2 mg/ml of solution or 2 mg/g fetuin cream. Sufficient unlabelled fetuin A was added to the solution and cream to bring the total fetuin A concentration up to 40 mg/ml or gram.

### Burn Injury

The porcine burn model has advantages over the lamb model as porcine skin is more similar to human skin and has less hair and no lanolin.

All animal experiments were approved by the University of Queensland animal ethics committee. Six 20 kg pigs (approximately 9 weeks post partum) were scalded under general anaesthesia. Water (400 mls) was placed in a 500 ml Schott bottle whose base had been removed with a diamond saw and replaced by cling wrap held in place by autoclave tape. The water was heated in a microwave oven to 92° C. (temperature was measured with a Dick Smith Q1437 digital thermometer) then the bottle was placed on the clipped side of the animal for 15 seconds. A single 8.5 cm diameter wound was placed on each side of the animal. Each animal received a different treatment these being:

1. Treatment of intact skin with an aqueous solution of 2 mls of Oregon green labeled bovine fetuin (40 mg/ml in sterile phosphate buffered saline pH 7.4).

2. Treatment of intact skin with an aqueous gel of 2 g of Oregon green labeled bovine fetuin (40 mg/g in Solosite™ wound gel).

3. Injection 2 mls of Oregon green labelled aqueous bovine fetuin (40 mg/ml in sterile phosphate buffered saline pH 7.4) over the wound surface as 50 ul aliquots at a depth of 1.5 mm.

4. Injection 2 mls of Oregon green labeled aqueous bovine fetuin (40 mg/ml in sterile phosphate buffered saline pH 7.4) subdermally and placement of an osmotic pump containing fetuin solution of the same concentration below the skin with a subsurface canula attached to deliver fetuin to the centre of the wound at a rate of 12 ul per hour)

The wounds of the above four animals were dressed with Melolin™ and Sleek™ (Smith and Nephew) and the pig was woken up and returned to its pen. These pigs were euthanized 4 hours post treatment and tissue sampled and stored for frozen sections.

5. Wounds were dressed with Melolin™ and Sleek™ (Smith and Nephew) and the pig was woken up and returned to its pen after wounding. The animal was re-anaesthetized 24 hours post burn and the wound debrided to punctuate bleeding. The wound was then treated with 2 mls (40 mg/ml) of labelled fetuin A in PBS and the animal was euthanized 4 hours post treatment application and samples taken for frozen sectioning.

6. Wounds were dressed with Melolin™ and Sleek™ (Smith and Nephew) and the pig was woken up and returned to its pen after wounding. The animal was re-anaesthetized 24 hours post burn and the wound debrided to punctuate bleeding. The wound was then treated with 2 gms of labelled fetuin A in Solosite gel and the animal was euthanized 4 hours post treatment application and samples taken for frozen sectioning.

Sections of 4 μm thickness were taken and examined under an EP600 epifluorescent microscope (Nikon) to determine the extent of fetuin A penetration in each of the carriers. Fluorescent blue (DAPI) and green (Oregon green) images were captured with a Spot RT™ slider cooled CCD camera and overlayed with ImagePro™ (Media Cybernetics) image analysis software.

Topical application of this fetuin in an aqueous cream enables penetration to the base of the hair follicles in the ovine model but there are relatively few follicles in pig skin. Full utilization of the properties of the protein such as modification of macrophage response would necessitate deep penetration into the wound. In the porcine burn model either direct injection into the wound or debriding the wound then applying fetuin A as an aqueous solution or a water based gel showed significant penetration into the wound bed when examined 4 hours post treatment. The methods and protocols described herein or routine methods known to those of skill in the art are used to optimise administration.

Fetuin A compositions can coat the epithelial layers and penetrate into the wound bed in a debrided deep partial thickness burn. This property, together with the above mentioned property of accelerating wound healing shows that fetuin will be beneficial in treating dermal and in particular burn injuries. Furthermore, debrided dermal wounds facilitate better penetration of topical agents than non-debrided wounds and enhanced healing.

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

TABLE 1
SUMMARY OF SEQUENCE IDENTIFIERS

| SEQ | |
|---|---|
| ID NO: | DESCRIPTION |
| 1 | AHSG cDNA encoding human fetuin Accession No. |
| | NM_001622 |
| 2 | Amino acid sequence of human fetuin |
| 3 | AHSG gene for human fetuin Accession No. AB038689 |
| 4 | AHSG cDNA encoding chimpanzee fetuin Accession No. |
| | NM_001009098 |
| 5 | Amino acid sequence of chimpanzee fetuin |
| 6 | AHSG cDNA encoding mouse fetuin Accession No. |
| | NM_013465 |
| 7 | Amino acid sequence of mouse fetuin |
| 8 | AHSG cDNA encoding rat fetuin Accession No. NM_012898 |
| 9 | Amino acid sequence of rat fetuin |
| 10 | AHSG cDNA encoding bovine fetuin Accession No. |
| | NM_173984 |
| 11 | Amino acid sequence of bovine fetuin |
| 12 | AHSG cDNA encoding sheep fetuin Accession No. |
| | NM_001009802 |
| 13 | Amino acid sequence of sheep fetuin |
| 14 | AHSG cDNA encoding pig fetuin Accession No. X56021 |
| 15 | Amino acid sequence of pig fetuin |
| 16 | AHSG cDNA encoding guinea pig fetuin Accession No. |
| | AB006443 |
| 17 | Amino acid sequence of guinea pig fetuin |

TABLE 2

| | σ1 | σ2 | σ3 | σ4 |
|---|---|---|---|---|
| Lamb | | | | |
| αSMA | 332 | 43.82 | 202.1 | 994.26 |
| TGF-β1 | 63.68 | 67.4 | 430.6 | |
| Fetus | | | | |
| αSMA | 40.56 | 14.68 | 7.16 | 132.19 |
| TGF-β1 | 44.17 | 124.9 | 31.33 | |

TABLE 3

| | | | | | |
|---|---|---|---|---|---|
| day 0 | STD1 | day 1 | STD2 | day 2 | STD3 |

Thyroglobulin

| | | | | | | |
|---|---|---|---|---|---|---|
| DMEM | 0.630411 | 0.157481 | 0.516419 | 0.216246 | 0.499119 | 0.233464 |
| 10% FCS | 0.64368 | 0.166653 | 0.485472 | 0.296835 | 0.021459 | 0.065362 |
| t 2.1 | 0.658655 | 0.168259 | 0.41297 | 0.170085 | 0.494102 | 0.202901 |
| t 4.2 | 0.642818 | 0.191148 | 0.577203 | 0.179089 | 0.539291 | 0.226667 |
| t 8.4 | 0.666859 | 0.167559 | 0.574242 | 0.217261 | 0.59095 | 0.191017 |
| t 16.8 | 0.6152 | 0.144082 | 0.598146 | 0.196137 | 0.613156 | 0.191956 |

TGFB-R inhibitor

| | | | | | | |
|---|---|---|---|---|---|---|
| DMEM | 0.667678 | 0.113696 | 0.575152 | 0.130818 | 0.564111 | 0.139049 |
| 10% FCS | 0.676729 | 0.123488 | 0.387112 | 0.193914 | 0.071008 | 0.143611 |
| 6.25 | 0.677882 | 0.103312 | 0.547328 | 0.143072 | 0.486597 | 0.151838 |
| 12.5 | 0.675059 | 0.133312 | 0.551466 | 0.139303 | 0.483857 | 0.152187 |
| 25 | 0.664302 | 0.105033 | 0.534102 | 0.116105 | 0.468815 | 0.138205 |
| 50 | 0.63715 | 0.113582 | 0.553998 | 0.124429 | 0.497981 | 0.120427 |

Fetuin

| | | | | | | |
|---|---|---|---|---|---|---|
| DMEM | 0.687636 | 0.153398 | 0.592272 | 0.155599 | 0.570048 | 0.167924 |
| 10% PCS | 0.695724 | 0.187616 | 0.427872 | 0.252324 | 0.067032 | 0.139203 |
| 3 | 0.760173 | 0.14742 | 0.426512 | 0.257263 | 0.029466 | 0.080593 |
| 6 | 0.73317 | 0.149242 | 0.393815 | 0.249299 | 0.014408 | 0.069493 |
| 12 | 0.723086 | 0.167118 | 0.369865 | 0.270864 | 0.000259 | 0.000461 |
| 24 | 0.672436 | 0.119545 | 0.380552 | 0.174477 | 0.043479 | 0.116159 |

### BIBLIOGRAPHY

Adzick N. S. et al., 1985, J. Pediatr. Surg., 20(4):315-319.
American Burn Association, 2003, J. Burn. Care Rehabil., 24(5):269-274.
Arora P. D. et al., 1999, Am. J. Pathol., 155(6):2087-2099.
Armstrong J. R. et al., 1995, Dev. Biol., 169(1):242-260.
Armstrong J. R. et al., 1997, Ontological wound healing studies of Monodelphis domestica from birth to adulthood. In: Saunders N, Hinds L, editors. Marsupial Biology: recent research, new perspectives. Sydney, Australia: University of New South Wales Press Limited; 254-271.
Atherton and Shephard, Peptide Synthesis. In Nicholson ed., Synthetic Vaccines, published by Blackwell Scientific Publications, Chapter 9.
Banine F. et al., 2000, Eur. J. Biochem., 267:1214-1222.
Brown W. M. et al., 1992, Eur. J. Biochem., 205(1):321-331.
Brown W. M. et al., 1997, Protein Sci., 6(1):5-12.
Bullard K. M. et al., 2003, World J Surg 27(1):54-61.
Burrington J. D., 1971, J. Pediatr. Surg., 6(5):523-528.
Burton et al., 1991, Proc. Natl. Acad. Sci. U.S.A., 88:10134.
Bull J. P. et al., 1954, Ann. Surg., 139(95):269-274.
Cass D. L. et al., (1997) J. Pediatr. Surg., 32(7):1017-1021; discussion 1021-102.
Chamberlain J. et al., 1995, J. Anat., 186(Pt 1):87-96.
Chaudhary et al., 1990, Proc. Natl. Acad. Sci. U.S.A., 87:1066-1070.
Clackson et al., 1991, Nature, 352:624.
Cohen J. (1960) Educ. Psychol. Meas., 20:37-46.
Colwell A. S. et al., 2003, Front Biosci., 8: s1240-1248.
Demetriou M. et al., 1996, J. Biol. Chem., 271(22):12755-12761.
Desmouliere A. et al., (1993) J. Cell Biol., 122(1): 103-111.
Desmouliere A. et al., (1995) Am. J. Pathol., 146(1):56-66.
Deutsch H. F., 1954, J. Biol. Chem., 208(2):669-678.
Diegelmann R. F. et al., (1981) Plast. Reconstr. Surg., 68(1):107-113.
Dugina V. et al., 2001, J. Cell Sci., 114(Pt 18):3285-3296.
Dziegielewska K. M. et al., 1980, J. Physiol., 300:441-455.
Dziegielewska K. M. et al., 1987, Cell Tissue Res., 248(1):33-41.
Dziegielewska K. M. et al., 1992, J. Comp. Physiol. [B] 162(2):168-171.
Ellington and Szostak, 1990, Nature, 346:818.
Erickson et al., 1990, Science, 249:527-533.
Estes J. M. et al., (1997) Differentiation, 56(3):173-181.
Ferguson M. W., 1994, J. Interferon Res., 14(5):303-304.
Ferguson M. W. et al., 2004, Philos. Trans. R. Soc. Lond. B. Biol. Sci., 359(1445):839-850.
Firth J. D. et al., 2004, J. Invest. Dermatol., 122(1): 222-231.
Fodor et al., 1991, Science, 251:767.
Fraser J. F. et al., 2005, Wound Repair Regen., 13(2): in press.
Gabbiani G., 2003, J. Pathol., 200(4):500-503.
Greenfield E. et al., 1996, Crit. Care Nurs. Clin. North Am., 8(2):203-215.
Greenhalgh D. G., Wound Healing. In Herndon D, ed. Total Burn Care, Vol. 1. London: W.B. Saunders, 2002. pp. 523-535.
Huang S. S. et al., 1997, Journal of Biological Chemistry, 272(43):27155-27159.
Hodgson, 1991, Bio/Technology, 9:19-21.
Hoogenboom et al., 1991 Nucleic Acids Res., 19:4133.
Huston et al., 1988, Proc. Natl. Acad. Sci. U.S.A., 85:5879-5883.
Huang J. S. et al FASEB J (21 Jun., 2002) 10:1096/fj.02-0103fje.
Igarashi A. et al, (1996) J. Invest. Dermatol., 106(4):729-733.
Jersmann H. P. et al, 2003, Clin. Sci. (Lond), 105(3):273-278.
Johnson et al “Peptide Turn Mimetics” in Biotechnology and Pharmacy, Pezzuto et al, Eds., Chapman and Hall, New York, 1993
Kang et al., 1991, Proc. Natl. Acad. Sci. U.S.A., 88:4363.
Kumta S. et al., 1994, Br. J. Plast. Surg., 47(5):360-368.
Lai P. C. et al., 1981, J. Reprod. Fertil., 63(1):53-60.
Leite-Browning M. L. et al., 2002, Int. J. Oncol., 21(5):965-971.
Li, F. et al., 2000, Exp Cell Res., 258(2): 245-253.
Longaker M. T. et al., 1991, J. Surg. Res., 50(4):375-385.
Longaker M. T. et al., 1994, Ann. Surg., 219(1):65-72.
Lorenz H. P. et al, 1995, Plast. Reconstr. Surg., 96(6):1251-1259; discussion 1260-1261.
Lowman et al., 1991, Biochemistry, 30:10832.
Moritz A, et al., (1947) Am. J. Pathol., 23:695-720.
Nie Z., 1992, Am. J. Physiol., 263(3 Pt 1):C551-562.
O'Kane S. et al., 1997, Int. J. Biochem. Cell Biol., 29(1):63-78.
Pedersen K. et al., 1944, Nature, 3914:575.
Roberts A., 1996, The Molecular and Cell Biology of Wound Repair, (2nd Edition): 275-308.
Shah M. et al., 1992, Lancet., 339(8787):213-214.
Shah M. et al., 1994, J. Cell Sci., 107 (Pt 5):1137-1157.
Shah M. et al., 1995, J. Cell Sci., 108 (Pt 3):985-1002.
Shakespeare P. G., (2001) Burns, 27(8):791-792.
Summerton and Weller, 1997, Antisense and Nucleic Acid Drug Development, 7:187-195.
Szweras M. et al., 2002, J. Biol. Chem., 277(22):19991-19997.
Tajirian T. et al., 2000, J. Cell. Physiol., 185(2):174-183.
Thiesen and Bach, 1990, Nucleic Acids Res., 18:3203.
Tredget E. E. et al., 1998, Plast. Reconstr. Surg., 102(5):1317-1328; discussion 1329-1330.
Tuerk and Gold, 1990, Science, 249:505.
Wang H. et al., 1998, Proc. Natl. Acad. Sci. USA, 95(24):14429-14434.
Wang R. et al., 2000, Wound Repair Regen., 8(2):128-137.
Wells, 1991, Methods Enzymol, 202:2699-2705.
Whitby D. J. et al., 1991, Dev. Biol., 147(1):207-215.
Whitby D. J. et al., 1991, Development, 112(2):651-568.
Whitby D. J. et al., 1991, J. Cell. Sci., 99(Pt 3):583-586.
Williams W. G., Pathophysiology of the burn wound. In Herndon D, ed. Total Burn Care, Vol. 1. London: W.B Saunders, 2002. pp. 514-522.
Wolf S. E. et al., 1997, Ann. Surg., 225(5):554-565; discussion 565-569.
Yachnin S., 1975, J. Exp. Med., 141(1):242-256.
Zawacki B. E., 1974, Surg. Gynecol. Obstet., 139(6):867-872.

1. A method for treating a dermal injury in a subject, comprising administering to the subject an effective amount of a fetuin polypeptide or an agent from which a fetuin polypeptide is producible.

2. The method of claim 1, wherein the dermal injury is a burn injury.

3. The method of claim 1 or 2, wherein the burn injury is a first or second degree burn injury.

4. The method of claim 1 or 2, wherein the burn injury is a third degree burn injury.

5. The method of claim 1 or 2, wherein the burn injury is a thermal burn injury.

6. The method of claim 1, wherein administration is local administration.

7. The method of claim 6, wherein the local administration is topical administration.

8. The method of claim 1, wherein the method comprises debriding the dermal injury to remove devitalised tissue.

9. The method of claim 8 wherein the dermal injury is debrided using any one or more of biological, chemical, enzymatic, mechanical or surgical debridement.

10. The method of claim 1, wherein the fetuin polypeptide or the agent from which the fetuin polypeptide is producible is applied to or otherwise associated with a medical device or medical material.

11. The method of claim 10, wherein the medical material is a suture, substitute skin, or dressing.

12. The method of claim 1, wherein the agent is a cell.

13. The method of claim 12, wherein the cell is a syngeneic cell.

14. The method of claim 11, wherein the cell is a dermal cell or a dermal cell progenitor.

15. The method of claim 12, wherein the cell is a genetically modified cell.

16. The method of claim 14, wherein the dermal cell is an epidermal cell.

17-29. (canceled)

30. The method of claim 14, wherein the dermal cell is selected from keratinocytes, melanocytes and fibroblasts or their progenitors.

31. The method of claim 1, wherein the fetuin polypeptide comprises all or part of the amino acid sequence set forth in SEQ ID NO: 2, 5, 7, 9, 11, 13, 15, and 17 or of a sequence having at least 60% sequence identity to the amino acid sequence set forth in any one of SEQ ID NO: 2, 5, 7, 9, 11, 13, 15, and 17.

32. The method of claim 1, wherein the agent from which the fetuin polypeptide is producible comprises a nucleotide sequence that encodes all or part of the amino acid sequence set forth in SEQ ID NO: 2, 5, 7, 9, 11, 13, 15, and 17 or of a sequence having at least 60% sequence identity to the amino acid sequence set forth in any one of SEQ ID NO: 2, 5, 7, 9, 11, 13, 15, and 17.

33. The method of claim 1, wherein the agent from which the fetuin polypeptide is producible comprises all or part of the nucleotide sequence set forth in any one of SEQ ID NO: 1, 3, 4, 6, 8, 10, 12, 14 and 16, or a sequence having at least 60% sequence identity to any one of SEQ ID NO: 1, 3, 4, 6, 8, 10, 12, 14 and 16, or a sequence that hybridizes to any one of SEQ ID NO: 1, 3, 4, 6, 8, 10, 12, 14 and 16 or to a complementary form thereof under at least medium stringency conditions.

34. The method of claim 1, wherein the fetuin polypeptide or the agent from which the fetuin polypeptide is producible is prepared with a pharmaceutically acceptable carrier, diluent and/or excipient.

35. The method of claim 1 or 34, wherein the fetuin polypeptide or the agent from which the fetuin polypeptide is co-administered with an ancillary wound healing agent.

36. The method of claim 35, wherein the ancillary wound healing agent is selected from cytokines, keratinocyte growth factors, platelet-derived growth factors, fibroblast growth factors, epidermal growth factors, neu differentiation factor, insulin-like growth factors and agents that modulate transforming growth factors.

37. The method of claim 35, wherein the ancillary wound healing agent modulates the level or functional activity of one or more members of the TGF-□ family.

38. The method of claim 35, wherein the ancillary wound healing agent is a non-fibrotic TGF-β molecule.

39. A kit comprising a fetuin polypeptide or an agent from which a fetuin polypeptide is producible and a debridement agent.

40. The kit of claim 39, wherein the debridement agent is selected from a biological, chemical, enzymatic or mechanical debridement agent.

41. The kit of claim 39, further comprising an ancillary wound healing agent.

42. The kit of claim 41, wherein the ancillary wound healing agent is selected from cytokines, keratinocyte growth factors, platelet-derived growth factors, fibroblast growth factors, epidermal growth factors, neu differentiation factor, insulin-like growth factors and agents that modulate transforming growth factors.


SEQUENCE LISTING (17 sequences)

SEQ ID NO: 1 - DNA, 1104 bp, Homo sapiens
  CDS (1)..(1101)
atgaagtccctcgtcctgctcctttgtcttgctcagctctggggctgccactcagccccacgtggcctagggctgatttatagacaaccgaactgcgatgatccagaaactgaggaagcagctctggtggctatagactacatcaatcaaaaccatccttggggatacaaacacaccttgaaccagattgatgaagtaaaggtgtggcctcggcagccctccggagagctgtttgagattgaaatagacaccctggaaaccacctgccatgtgctggaccccacccctgtggcaagatgcagcgtgaggcagctgaaggagcatgctgtcgaaggagactgtgatttccagctgttgaaactagatggcaagttttccgtggtatacgcaaaatgtgattccagtccagactcagccgaggacgtgcgcaaggtgtgccaagactgccccctgctggccccgctgaacgacaccagggtggtgcacgccgcgaaagctgccctggccgccttcaacgctcagaacaacggctccaattttcagctggaggaaatttcccgggctcagcttgtgcccctcccaccttctacctatgtggagtttacagtgtctggcactgactgtgttgctaaagaggccacagaggcagccaagtgtaacctgctggcagaaaagcaatatggcttttgtaaggcaacactcagtgagaagcttggtggggcagaggttgcagtgacctgcacggtgttccaaacacagcccgtgacctcacagccccaaccagaaggtgccaatgaaacagtccccacccccgtggtggacccagatgcacctccgtcccctccacttggcgcacctggactccctccagctggctcacccccagactcccatgtgttactggcagctcccccaggacaccagttgcactgggcacactacgacctgcgccacaccttcatgggtgtggtctcattggggtcaccctcaggagaagcgtcgcacccccggaaaacacgcacagtggtgcagcctagtgttggtgctgctgctgggccagtggttcctccatgtccggggaggatcagacacttcaaggtctag

SEQ ID NO: 2 - protein, 367 aa, Homo sapiens
MKSLVLLLCLAQLWGCHSAPRGLGLIYRQPNCDDPETEEAALVAIDYINQNHPWGYKHTLNQIDEVKVWPRQPSGELFEIEIDTLETTCHVLDPTPVARCSVRQLKEHAVEGDCDFQLLKLDGKFSVVYAKCDSSPDSAEDVRKVCQDCPLLAPLNDTRVVHAAKAALAAFNAQNNGSNFQLEEISRAQLVPLPPSTYVEFTVSGTDCVAKEATEAAKCNLLAEKQYGFCKATLSEKLGGAEVAVTCTVFQTQPVTSQPQPEGANETVPTPVVDPDAPPSPPLGAPGLPPAGSPPDSHVLLAAPPGHQLHWAHYDLRHTFMGVVSLGSPSGEASHPRKTRTVVQPSVGAAAGPVVPPCPGRIRHFKV

SEQ ID NO: 3 - DNA, 10327 bp, Homo sapiens
ctccagggcttatgctatcatcctgccttcatagttgggattacaggtttgtgccaccttaccacactttattcaatttttcttttttttgagtcggagtcccactccgttgccaggctggagtgcagtggcgcaatcttggctcactgcaacctccacctcctgggttcaagtgattctcctgtctcggcctcccgagtaggtgggactacaggcgcgcgccaccacgcccagctaatttttgtatttttagaagagacagggtttcactgtgttggtcaggttgggctcaatctctcggcctcgtgatctgcccacctcagcctcccaaagtgctggaattacaggtgtgaaccaccgtgcccagcccttcaatatttttaaagattatataaagaaaagcatagcttccacacacagttcaggagtcagggccctgagtgaaatggagtataaggtgatggaagattgcccctggctttgaggatcatcttgctggatgaccagagtttgtttttcatctggaagaatcatggtccccctcttggagagaacctggagtcctggccacaccttctgagcttctgacattaagattcggtgtggtgtgtgagcagctgggagtaaatgtgagaacagtcctgccaactaccttctctgccccatcaccactcacttcacctccctcaccccgattctcaccccagaaaatcaatattaagcttccctttggaggggcccctcttctttcttttttttttaaccagttttttttttctctgaagcaatatcaaacattagaacaaattcacccacacacaatgatggcctctaaatatttgaagaccatgttggtctccatgagagggcttctttgcttcagactaaaccaaaccaggtccttgtgtgatatttcccaagcctctctttatcctgccaaccctccttggatgtcacacttggaattgtcaagggtcacttaataggcagtgcctggagtcaacactggccctctgttctggccagaaagacacactgtaattctgctgagtctgcaaagaatgttcaaggggtatttgtgtccgtagctgctcgtgcagcatctcagcctgcggtgtcagactcgaacccagagctgtgtcaaaagagtcaggactcccctctgctgcccttgtgcactggcaggggcactaggttactagtgctaggagactctggtcagtgcccatctgtgctgtcacatgcagggccctggcagtgaggtctggagaaggaagagaaggggcagggaaccccacacgttttgctggtcatttcccaccgtggtcctgccacgggaacccccttcttccacctccattccccactttaggcagaaacatctctcatcctttttcttttctccaagaatcttcccccaaaatcttggcatctcctatacacatctgtacctttgctatatctgtgggccttgttctccctgtaggactcctcttggaaagctaactccccctgacatgggagaattttatcaaattgctgagtatcctcccttccctctcacagaccccatggcatgctcagatcacagtagaagacatttcctctgctgccaaacccatggcactctgaggctgactgtgtccacctcattccctcagctgtcttctctttgctgctattaccatgttccaagcagactttggagcatctcccccacagcagcatggactttggcagatttcttggggaccagcgatgtcctaacctgtttgcttttccagggctgatgtttgcagggtgtttttttttttcttttgaaccaaagcagaaatcatcctgtatccttatgcaattcttccggcaggctccaacagataaataaagcccaccaccctccatgggtctacctttcccagcagagcacctgggttggtcccgaagcctccaaccacctgcacgcctgccagggcctctctggggcagccatgaagtccctcgtcctgctcctttgtcttgctcagctctggggctgccactcagccccacatggcccagggctgatttatagacaaccgaactgcgatgatccagaaactgaggaagcagctctggtggctatagactacatcaatcaaaaccttccttggggatacaaacacaccttgaaccagattgatgaagtaaaggtgtggcctcaggtaagtggacctgctgtctatgagctgaaataatgtgtacatggagctcaatcaggtgcctcaaaaaatcaccatcacccagtgcaaatgaaaccacagaggagtaaattctctgatttcttcccaggagtgagggaaggggcaggcagagggcaggagaggagacattctgtatggcagtcatgggtgtcaggagggagctgggtggggtgtgaggtggtgtgcaggagaaaaagtgcttcaaatggtagtgtgcagatcacagacagaaagtgtaacttgctggaaaaactaggacccaagagaccagctcctagttgccaagttaccactggctgaaaatcacgtatctgtctttggtttggtttctctctaacaaagactgagaatgaataaaactagcatctggcagatgcctactatatgccaggcccattcacatagattatctcatttactctttcccggtcctgcctcctggtgctgtgtggtacatatattgttcttgtcttacccaagaggagaccaaggctctgttgtgtgtgtgtgtgcagtttttttgttttttgtttttgtctttttttttttggtccaaaatcatataattactaagtcttcaggctgggatttgattccatatctgtgttccttcttctacacaaactgcctcccaaagagagttacccacatcccagagagaagtcttggcataaacacaattcacctcctcacacactagacaggaaaccaacgcagcttgaagccagtgacaagaaaaatcaagctggaaatatgcctcggggatcagtcaagagatttggagaggtggaaagaagctgtctgcctactgcctgttttgaaattagatttatttctgattaaggacaattctttcagcaaatatgtattacaagccttccttggacaagaaccagagatattaggttgaaccatataaaactgccatttttctatatcaaaagcaaccaaatattggccgttttaatggttcaacctaatacagtggtgaaaaaggcataatatgtgcccacaagagcttacaatctaggttggaaaataaggtcaacaacaggaagcctggaccgactgacgactgccatccgtctcacaaagagacaaaatatttgaaatcaggattgctccggatggattttaagagtgctgcagccatattaaagcacagtggtggttaggaggaaacgctgatcaagtcaggggaaatgaacacgcaacacgcacatctgagggaaaaggtaatcatgaatgggcattgtgacttttactaaaggcagagcttcagagttggttcccttgagaaacccaggtgtacccggttcctgttcgccagagctgtgaacgctttcaggcagtcactctgggcacacctggacatcataaaatgcggaacttctcccaggggaggggatgctgaggcttcaggtactagtgaatcaggcagaaccaatgagaggcaaacagagctgggctgagaggagaaaaggcatacttgtaccttctggatttttcaggcttcgaagacaagatacagaaacaggtgaactcacaagaatatctccaaggattgttgcaagctccctcgtgtctacactagtgacatccagtttcctgtcagagggagacatgcccttccccattatcgccagcagggggaagtagagagcagcatcgttgcatgccggcacctgctgcacaagccaagacaaaggaaaaaccaaggacaacagcagcaaaaacctctaggagggaaaagaaaacggaggaaggaaggaaagcaaataatgaaaaggaagaaagaaagaaggagagggagggatagaggagattaaaaaaagacagtaagatattaccctacaccaccttattttgcagcttgtctgagaaaaatccaaacttgcattttccaaagcactgcttgaagagtgaaatcttaaaaaattaaataaataataaatacaaataagtgttaacacccatttgtagttttcaaatagagctcagagtgagggctgtggctccatcgacttgttcaagcccaggaccccgtctgctttgcgagcatcatctggtgcttccttaatcaacagacgaagaccagacaagccctggtcattgtcctgcccacaggccagttcagagctagacggagttgcagactgacagtaagaatgacatttccctcacctctccaaaagcggggtgctctcaagcccaatgagggcgcataccgtggaccgcaccacaggatcaggggaataggttgctcacggcttcactctttgtctccacagcagccctccggagagctgtttgagattgaaatagacaccctggaaaccacctgccatgtgctggaccccacccctgtggcaagatgcagcgtgaggcagctgaaggagcatgtgagtacccttcttaggatgactgtaggtggcccttcggccagctccaccgattcacccagcgtctcagcctgccttcttggctagccagggtgcagtttctaaaattgccatttgtggccgagcgcagtggctcatgcctataatctcagcactttgggaggctgaggcaagtggatcgcctgaggtcaggagttcaagaccagcctggccagtatggtgaaaccccatctctactaaaaatacaaaaattagctggacgtggtgacgggcacctgtaaatcccagctcctcgggaggctgaggcaggagaatcgcttgaacccgggaggtggaggttgcagtgagccaagatcctgccattgcactccagcctgggcaacaacagtgaatctctatctcgaaataataataataatcatcatcatcataaataaaattgccatttgatgccacttgccctggggctgatttttacaagcgtttaactatatcgttgtatccctgaaagcagagagtgccatgtttcagtattacccagcaaaggcgattttgcaagggtcacctttgacagccgtgcctggagggagcctgcccggggtgcgaaggggaagggcagccatcctcacgtgggtttctttctccaggctgtcgaaggagactgtgatttccagctgttgaaactagatggcaagttttccgtggtatacgcaaaatgtgattccagtccaggtacagatgactattcttattctcattttttccttgtagagaaagtggggaagggatctgaataattttcaacttaagtagttctagcagctttgtcggtgaggaaaaggagaagccaaatttcctgggttctgggatttttaaaattgtgttttaagaagctactcttggcctggtgcggtggctcacgcctgtaatccacccacccgaggcaggtggatcacctgaagtcagaagttcgagaccagcctggccaacatagtgaaacccccatctctactaaaaatacaaaaatgtggtggtgctcgcctgtaatcccagctactagggaggctgaggcaggagaatcgcttgaacctgggaggcagaggtggcagtgggccgagatcgcaccactgcactccagcctgagtgacacagagtgagaccctgtctcaaaaaaataagaagttattcttactggaagtgaaaattgcctcgtgatgataagagctcctgcagaaatgtcagcatagcaaaagccttttgaaggtttagtaagaagcagagaaagtgcctgaagctatctggggaatgccttagcccttgctaacgcagcagagctggggccatgccagggagaatggctgcccacatcctggtttcctctctccgagcagactcagccgaggacgtgcgcaaggtgtgccaagactgccccctgctggccccgctgaacgacaccagggtggtgcacgccgcgaaagctgccctggccgccttcaacgctcagaacaacggctccaattttcagctggaggaaatttcccgggctcagcttgtggtaaagactgagattcttttgacaggttgggcagttcggtggcacttcgggaatgtactgtacgtggtggagcgggaggcagggaagaacaggcgcaggggcagcgatgagaaagcaaggagagggttgtttggaaagggaagaaagcatcctaagggggtatgaggctcctgagtgtcatgaggaccccaacaccctcagcgcctcccccatgctgagccactgtaacgtccagcagccacagctgccggcaggtacatccccactccctccgttccagctaaaaccaaagctcagtgtcagctggtagagttgcccacgtcggccagaagcactcactgtaaatttgctgggctccagtaccacccatctccgctgaacatctgccacagactcgtaattaatactcacttgtgctgacaagcttataatggcaagatcttaaaatgcctttcgagtcactggagaaaacatctcattgtactgtgggtggtttagcacattggaattcaacagaattcaaatgtttaagaaaatgtattctggatatcagccatggccatacttggaaatacgctagtatagacggcaattctattaatcagaatatgtgattctcagaacatccccaccccagactacaccaaataacagatattttattgtgtccatatgctccaactactttaaaaaagaaaagctcaagtgatatcttccatactttcatctaaatcttttcatttgagcctgctctatgaaacaggtggaagaggtattaatctcttcactttcccaccctatcttggaataacctgaaccttgggtatcaagtgcagcccaagagtgagggctggggggaggcagggttcccactcctatcagtctaaggctggccttctgattccggtttcctatctgcaaactcacctccaccctgaaggaccggtgatggatacttgcccctcctacaaggaagacacaacccctacctctaaagcacaagcacttgagaacacaaccccatagcaactgccctatgtaagccattgagggacatgtcttctgggccgacgcatggtctgcatgaatggtgctccccgaaggaggctacttcccgctctccttctctgcccttttcattgtaagtcatctttcctcaagagcattttcatgtactcttctcagcccctcccaccttctacctatgtggagtttacagtgtctggcactgactgtgttgctaaagaggccacagaggcagccaagtgtaacctgctggcagaaaaggtgagtgggccgggaccttggggtgttaccactcggacagagctgtttgtggaacagaacatccttggatagtttgtatcttggggctgcagacagagaataacagtgcaaatcccctctccctgtggatcacggcaagccttcttttagggtgtcacctcatccctttaagagctgtcatcaaatcatctcacccactggaagcacatgaagttaggagaaagagagaggctatttgctaatgaagccaagtcacgcccacccactgggaatgtgaagtgcacattttctagacatataactctgatacaaaagctttcaagtccttgagccaataatgtacacttctaggattttagtctaaagaagtcatcagtggccaggcatgatggctcatgcctgtaattccagcactttgggaggccaagacgggtggatcgggaggtcaggagatcgagaccatcctggctaacatggtgaaaccccgtctctactaaaaatacaaaaaaattagccaggcttggtggtgagcgcctgtagtcccagctactcgggaggctgaggcaggagaatggtgtgaacccaggaggcagatgttgcagtaaactaagatcgtgccactgcactccagcctgggcaacagaacgagactctgtctcaagaaaaaaagaaaaagaaaaagaattcatcagtgacatttgacagaatatatctataaaaatgatttattatggatataaagagaccaaaaaagagagatctgtatgtccaacaggaaggtgtcattgaataatccatgcacatcagtaaatagaaaattgtgcagacactaaaaattgtgttttcaaggaataatgaatgatatgagaaaatgctattatggcaagtgaaaacacacaggatacaacatcgtatagtcacaatgatctcaatttttaaatcatatttaatagtattttaaaataagaaagaaatgcatcaatgttaacagtccttctttctaggccaccaccagaaagggattatgggtaatctctctcactctcaaagtatttctgtatttccatgttatatatagaatcatatacctcccacaagcagaaactataactttaagaaaaatggtttttcaaactaatttaaggttggcgcgtcaatgaaattgggggggatccatttttgaaattagttaaaataaatcctctttctctgtgggcagcaatatggcttttgtaaggcaacactcagtgagaagcttggtggggcagaggttgcagtgacctgcacggtgttccaaacacaggtaacagctccgtgaatattcttgcctacaccttcagaatacaatgaccccttcacatttatgcagtgcagtagtgatgacaggacatttgctctcccgtgcttctgaatctcacagtatgaaataacactggggtatgcggaatcatcaacaaatggaaggatattttagctatgcctttccctcccacgaactagtgactacagggaagaaccatcttactgtgtagttgacaaagccacctttttatttgtgggaggtgggagtggttttctgagttgcagagaccaggtggccagatctacctgttagctcccagtggctgcagcttcagatgacaaagagggtggcactgctgggcaagggtgagccataggtggggtgcttttactcattggacatatgtgtgtaagtccaccatcacaaagacaatcctagtgaggccggggccaacataggccagtcacccctccttgtaaccttgatgacaatcccttgtacttaggtaggtcctttcttgctagactctttgcaaataaaaatgtataatgtgaggaaattgggtgccagtgccacctgggcctgtgggttgtcttgcctgggaggaggaagcaaactaactgaaggaaatggtcctttttccagcccgtgacctcacagccccaaccagaaggtgccaatgaagcagtccccacccccgtggtggacccagatgcacctccgtcccctccacttggcgcacctggactccctccagctggctcacccccagactcccatgtgttactggcagctcctccaggacaccagttgcaccgggcgcactacgacctgcgccacaccttcatgggtgtggtctcattggggtcaccctcaggagaagtgtcgcacccccggaaaacacgcacagtggtgcagcctagtgttggtgctgctgctgggccagtggttcctccatgtccggggaggatcagacacttcaaggtctaggctagacatggcagagatgaggaggtttggcacagaaaacatagccaccattttgtccaagcctgggcatgggtggggggccttgtctgctggccacgcaagtgtcacatgcgatctacattaatatcaagtcttgactccctacttcccgtcattcctcacaggacagaagcagagtgggtggtggttatgtttgacagaaggcattaggttgacaacttgtcatgattttgacggtaagccaccatgattgtgttctctgcctctggttgaccttacaaaaaccattggaactgtgactttgaaaggtgctcttgctaagcttatatgtgcctgttaatgaaagtgcctgaaagaccttccttaataaagaaggttctaagctgaatgtggtcatgcttattgcgacttcatcccagctcccctcacatgcatagccttttaccccaacaaacacagtgtccctaatcaaaaccaaagtgaaaagagaaccaaaagagaacaaaaacctgctgtattgccagatacaggaaaaagtgagactaggatc

SEQ ID NO: 4 - DNA, 1104 bp, Pan troglodytes; identical to SEQ ID NO: 1
  CDS (1)..(1101)

SEQ ID NO: 5 - protein, 367 aa, Pan troglodytes; identical to SEQ ID NO: 2

SEQ ID NO: 6 - DNA, 1038 bp, Mus musculus
  CDS (1)..(1035)
atgaagtccctggtcttgctcctttgttttgctcagctctggggctgccaatccgctccacaaggtacaggactgggttttagagaattggcttgtgatgatccagaagcagagcaagtagctttgttggccgtggactacctcaataatcatcttcttcagggattcaaacaggtcttgaatcagatcgacaaagtcaaggtgtggtctcggcggcccttcggagtggtgtatgagatggaagttgacacactggagaccacttgccatgctttggaccccaccccgctggcaaactgttctgtgaggcagctgactgagcacgcggtggagggagactgtgacttccacatcctgaaacaagacggccagttcagggtgatgcacacccagtgtcattccaccccagactctgcagaggacgttcgtaagttgtgcccacggtgcccactcctgactccgttcaacgataccaacgtggtccacaccgtcaacactgccctggctgccttcaacacacagaataatggaacctattttaaactggtggagatttcccgggctcaaaatgtgcctctcccagtgtctactctggtggagtttgtaatagctgccactgactgtactgcaaaagaagtcacagatccagccaaatgcaacctgctggcagagaagcaacatggcttctgcaaggcaaatctcatgcataatcttggtggggaagaagtttcagtggcctgcaagttattccaaacacagccccagccagccaatgccaacgcagtaggtcccgtacccacagcgaatgcagccctaccagctgacccacctgcatctgtggtggtgggacctgtggtggttccacgaggactttcagaccaccgaacttaccacgacctacgccacgccttctctcctgtggcctcggtggagtcggcctcgggagaaactcttcattctcctaaggtgggccagcctggtgctgctggtccagtgtcccccatgtgcccagggaggatcagacacttcaaaatctag

SEQ ID NO: 7 - protein, 345 aa, Mus musculus
MKSLVLLLCFAQLWGCQSAPQGTGLGFRELACDDPEAEQVALLAVDYLNNHLLQGFKQVLNQIDKVKVWSRRPFGVVYEMEVDTLETTCHALDPTPLANCSVRQLTEHAVEGDCDFHILKQDGQFRVMHTQCHSTPDSAEDVRKLCPRCPLLTPFNDTNVVHTVNTALAAFNTQNNGTYFKLVEISRAQNVPLPVSTLVEFVIAATDCTAKEVTDPAKCNLLAEKQHGFCKANLMHNLGGEEVSVACKLFQTQPQPANANAVGPVPTANAALPADPPASVVVGPVVVPRGLSDHRTYHDLRHAFSPVASVESASGETLHSPKVGQPGAAGPVSPMCPGRIRHFKI

SEQ ID NO: 8 - DNA, 1059 bp, Rattus norvegicus
  CDS (1)..(1056)
atgaagtccctggtcctgctcctttgttttgctcagctctggagctgccaatcagctccacaaggtgcagggctgggttttcgagaattggcttgtgatgacccggaaacagagcatgtagctttgatagccgtgcactacctcaataaacatcttcttcagggattcaggcagatcttgaatcagattgacaaagtcaaggtgtggtctcggcggcccttcggacaggtgtatgagttggaaattgacacgctggagacaacctgccacgctctggacccaacccccttagcaaattgttctgtgaggcaacaggccgagcatgcggtggagggagactgtgacttccacatcctgaaacaagatggccagttcagggtgctacacgcccagtgtcattccacaccagactctgcagaggacgttcggaagttttgcccacgatgcccaatcctgatccggttcaatgacaccaacgtggtccacactgtcaaaactgccctggctgccttcaatgcacagaataatggaacctattttaaactggtggagatttcccgagctcaaaatgtgcctttcccggtgtctactctcgtggagtttgtgatagctgcgactgactgtactggtcaggaggtcacagatccagccaaatgcaacctgctggcagaaaagcaatatggcttctgcaaggcgactctcatccacagacttggtggggaagaggtttcagtggcctgcaagttattccagacacagccccagccagccaatgccaaccctgcaggtcctgcacccacagtgggccaagctgcgcctgtagccccaccagctggcccacctgaatccgtggtggtaggacccgtggcagttcctctaggacttccagaccaccgaacccaccatgacctacggcatgccttctctcctgtggcttctgtggagtcggcctcaggagaagttcttcactcacctaaggtgggccagcctggtgacgctggtgctgctggcccagtggcccccctgtgcccagggagagtcagatacttcaagatctag

SEQ ID NO: 9 - protein, 352 aa, Rattus norvegicus
MKSLVLLLCFAQLWSCQSAPQGAGLGFRELACDDPETEHVALIAVHYLNKHLLQGFRQILNQIDKVKVWSRRPFGQVYELEIDTLETTCHALDPTPLANCSVRQQAEHAVEGDCDFHILKQDGQFRVLHAQCHSTPDSAEDVRKFCPRCPILIRFNDTNVVHTVKTALAAFNAQNNGTYFKLVEISRAQNVPFPVSTLVEFVIAATDCTGQEVTDPAKCNLLAEKQYGFCKATLIHRLGGEEVSVACKLFQTQPQPANANPAGPAPTVGQAAPVAPPAGPPESVVVGPVAVPLGLPDHRTHHDLRHAFSPVASVESASGEVLHSPKVGQPGDAGAAGPVAPLCPGRVRYFKI

SEQ ID NO: 10 - DNA, 1080 bp, Bos taurus
  CDS (1)..(1077)
atgaaatccttcgttctgctcttttgcctggctcagctctggggctgccactcgatcccgcttgacccggttgcaggttataaggaaccggcctgtgatgacccagacacagagcaagcagccttggctgccgtggactacatcaacaagcaccttcctcggggctacaagcacaccttgaaccagattgacagtgtgaaggtgtggccgaggcggcccacgggagaggtgtatgacattgaaatagataccctggaaaccacctgccacgtactggaccccacgcccctggcgaactgcagcgtgaggcagcagacgcagcacgcggtggaaggagactgcgatatccacgtgctgaaacaagatggccagttttccgtgctgtttacaaaatgtgattccagtccagattccgccgaggacgtgcgcaagttgtgcccagactgccccctgctggcgccactcaacgacagccgggtggtgcacgcagtggaggtcgcgctggctaccttcaatgccgagagcaacggctcctacttacagctggtggaaatttctcgggctcaatttgtgcctcttccagtttctgtctctgtggagtttgcagtggctgctactgactgtattgctaaagaagtcgtagatccaaccaagtgcaacctactggcagaaaagcaatatggcttctgtaaggggtcagtcattcagaaagctcttggtggggaggacgtcagagtgacttgcacgttgttccaaacgcagcctgtgattccgcagccccagcccgacggcgccgaggctgaggccccaagcgctgtgccggacgcagctgggcctacgccttctgcagctggcccgcccgtggcctccgtggtggtggggccaagcgtggtagcagttcccctgccgctgcaccgagcacactacgacttgcgccacactttctccggggtggcctcagtggagtcatcctcgggagaagcgttccacgtgggcaaaacacccatagtggggcagcctagcattcctggaggtccagtccgcctttgcccagggagaatcagatacttcaagatctag

SEQ ID NO: 11 - protein, 359 aa, Bos taurus
MKSFVLLFCLAQLWGCHSIPLDPVAGYKEPACDDPDTEQAALAAVDYINKHLPRGYKHTLNQIDSVKVWPRRPTGEVYDIEIDTLETTCHVLDPTPLANCSVRQQTQHAVEGDCDIHVLKQDGQFSVLFTKCDSSPDSAEDVRKLCPDCPLLAPLNDSRVVHAVEVALATFNAESNGSYLQLVEISRAQFVPLPVSVSVEFAVAATDCIAKEVVDPTKCNLLAEKQYGFCKGSVIQKALGGEDVRVTCTLFQTQPVIPQPQPDGAEAEAPSAVPDAAGPTPSAAGPPVASVVVGPSVVAVPLPLHRAHYDLRHTFSGVASVESSSGEAFHVGKTPIVGQPSIPGGPVRLCPGRIRYFKI

SEQ ID NO: 12 - DNA, 1095 bp, Ovis aries
  CDS (1)..(1092)
atgaaatccttccttctgctcttttgcctggctcagctctgtagctgccgctcgatcccgcttgacccgattgcaggttataaggaaccggcctgtgatgacccagacacagagcaagcagccttggcggccgtggactacatcaacaagcaccttcctcggggctacaaacacaccttgaaccagattgacagtgtgaaggtgtggccgaggcggcccacgggagaggtatatgacattgagatagataccctggaaaccacctgccacgtactggaccccacgcccctggtgaactgcagcgtgaggcagcagacggagcacgcggtggaaggagactgcgatatccacgtgctgaaacaagatggccagttttccgtgctgtttacaaaatgtgattccagtccagattccgccgaggacgtgcgcaagttgtgcccagactgccccctgctggccccgctcaacaacagccaggtggtgcacgccgcagaggtcgcgctggctaccttcaatgcccagaataacggctcctactttcagctggtggaaatttctcgggctcaatttgtgcctcttccaggttctgtctctgtggagtttgcagtggctgctactgactgtattgctaaagaggtcgtagatccaaccaagtgcaacctactggcagaaaagcaatatggcttctgtaaaggctctgtcattcagaaagctcttggtggggaagacgtcacagtgacttgcacactgttccaaacgcagcctgtgattccgcagccccagcccgaaggcgccgaagctggggccccaagcgctgtgccggacgcagctgtgcctgacgcagctgtgcctgcgccttctgcagccggcctgcctgtgggctccgtggtggcggggccaagcgtggtagcagttcccctgccgctgcaccgggcacactacgacttgcgccacaccttctccggtgtggcctcagtggagtcggcctcgggagaagcattccacgtggggaaaacacccatcgtggggcagcctagcgttcctggaggtccagtccacctttgcccagggagaatcagatacttcaagatctag

SEQ ID NO: 13 - protein, 364 aa, Ovis aries
MKSFLLLFCLAQLCSCRSIPLDPIAGYKEPACDDPDTEQAALAAVDYINKHLPRGYKHTLNQIDSVKVWPRRPTGEVYDIEIDTLETTCHVLDPTPLVNCSVRQQTEHAVEGDCDIHVLKQDGQFSVLFTKCDSSPDSAEDVRKLCPDCPLLAPLNNSQVVHAAEVALATFNAQNNGSYFQLVEISRAQFVPLPGSVSVEFAVAATDCIAKEVVDPTKCNLLAEKQYGFCKGSVIQKALGGEDVTVTCTLFQTQPVIPQPQPEGAEAGAPSAVPDAAVPDAAVPAPSAAGLPVGSVVAGPSVVAVPLPLHRAHYDLRHTFSGVASVESASGEAFHVGKTPIVGQPSVPGGPVHLCPGRIRYFKI

SEQ ID NO: 14 - DNA, 1089 bp, Sus scrofa
  CDS (1)..(1086)
ctcatcctgttcttttgccttgctcagctctggggctgccgtgctgtcccccatggcccgattctgggttacagggaaccggcctgtgatgacgtagaaacagagcaagcagccctggcggccgtggactacatcaataagcaccttcctcggggctacaagcacaccttgaaccaggttgacagcgtgaaagtgtggccaaggcggcccgcgggagaggtatttgacattgaaatagacacgctggaaaccacctgccatgtcctggaccccactcccctggcaaactgcagcgtgaggcagctgacagagcatgcggtggaaggagactgcgatttccacgtgctgaaacaggacggccagttctccgtgttatttgcaaaatgtgattccagtccagactcagctgaggatgttcacaaggtgtgtccaaactgccccctgctggccccgcttaatgacagccgggtggtgcacgctgcagaatcggcgctggctgccttcaacgcccagagcaacggctcctatttgcagcttgtggaaatttctcgggctcaacttgtgcctctttcagcttctgtctctgtggagtttgcagtagctgtcactgactgtgtggctaaagaggcatatagcccaaccaaatgcaacctgctggtggaaaagcaatatggcttctgtaaggggacagtcacagcgaaggttaatgaggaagatgttgcggtgacctgcacggtgttccaaacccagcctgtggtcctgcagccccaacctgccggtgctgatgcgggggccacccctgtggtggatgcagctgcaactgcatctcctctagctgacgtgcccgcagcttccctggtggtgggacccatggtggtggcagttcctccgggcatcccgccagtgcaccgatcacactacgacctgcgccactctttctcgggtgtggcctcagtggagtcagcctccggagaagcattccacgtggggaaaacacccaagggggcacagcctagcattcctgctgctgatggctcagttccagtggtccgcccttgccctgggagaatcagacacttcaagatctag

SEQ ID NO: 15 - protein, 362 aa, Sus scrofa
LILFFCLAQLWGCRAVPHGPILGYREPACDDVETEQAALAAVDYINKHLPRGYKHTLNQVDSVKVWPRRPAGEVFDIEIDTLETTCHVLDPTPLANCSVRQLTEHAVEGDCDFHVLKQDGQFSVLFAKCDSSPDSAEDVHKVCPNCPLLAPLNDSRVVHAAESALAAFNAQSNGSYLQLVEISRAQLVPLSASVSVEFAVAVTDCVAKEAYSPTKCNLLVEKQYGFCKGTVTAKVNEEDVAVTCTVFQTQPVVLQPQPAGADAGATPVVDAAATASPLADVPAASLVVGPMVVAVPPGIPPVHRSHYDLRHSFSGVASVESASGEAFHVGKTPKGAQPSIPAADGSVPVVRPCPGRIRHFKI

SEQ ID NO: 16 - DNA, 1077 bp, Cavia porcellus
  CDS (1)..(1074)
atgaagttctttgttttgttcctttgccttgttcagctctggggctgtcactcaaccccagttgtcttggggctggaggagaggaacccagcttgcgatgacccagagaccgaggcggcagccctggcaggtgtggactacctcaatcagcatgttcgttggggctacaagcacgtcttgaaccaaattgacaaagtcagagtgtggcctcggcggccctccggagaggtgtatgagcttgaattcgacacgctggagaccacctgccacgccttggacatcacgcccttggcgaactgctctgtgagaacggtgacccaacacgcggtggaaggggactgtgacatgcacgtgctaaaacaggacggccagttttccgtggtgtttgcaaaatgcgagtccactccagactccagggaggacgtgcgcaaagtgtgcccccagtgccccctgctgacgcctgtgaacaacaccaaggtggtgcacgcggccaatgccgccctggccgccttcaacgcgcagaacaacggctcccactttgagctgctggagatttcccgcgctcagctcgtgcctctcccagtttctacctatgtagagtttgcagtggtagctactgactgtgctgctgcagacggcacagatccagcctgctccgggccaccaaaaaagcaatatggcttctgcaaggcgacagtcgctgagaagcttggtggggaagaagtttcagtgacctgcacggtgttcccaacacagcctgtggcccccctgccccagccagatgcagcctcatcagcaaacccaccccctgcagccgacccagctgtatctccaccctcttcacctagtgtgcctgtggattctgtggcactgggacccaagagggtgttacttccaccccccgtgcaccgggaacactataacctgcgctacagcttcccagatgtggattcagcctcaggagaagcattcggcccaaggcagaagcccaaggtgacacatcctggcgtggccagtggtgttggcccagtgcctcctcccccatgccctgggaggatcagacacttcaagatctag

SEQ ID NO: 17 - protein, 358 aa, Cavia porcellus
MKFFVLFLCLVQLWGCHSTPVVLGLEERNPACDDPETEAAALAGVDYLNQHVRWGYKHVLNQIDKVRVWPRRPSGEVYELEFDTLETTCHALDITPLANCSVRTVTQHAVEGDCDMHVLKQDGQFSVVFAKCESTPDSREDVRKVCPQCPLLTPVNNTKVVHAANAALAAFNAQNNGSHFELLEISRAQLVPLPVSTYVEFAVVATDCAAADGTDPACSGPPKKQYGFCKATVAEKLGGEEVSVTCTVFPTQPVAPLPQPDAASSANPPPAADPAVSPPSSPSVPVDSVALGPKRVLLPPPVHREHYNLRYSFPDVDSASGEAFGPRQKPKVTHPGVASGVGPVPPPPCPGRIRHFKI